(19) United States
(12) Patent Application Publication
(10) Pub. No.: US 2009/0022803 A1
(43) Pub. Date: Jan. 22, 2009

(54) Oral Formulations For Delivery of Catecholic Butanes Including Ndga Compounds

(75) Inventors: Rocio Alejandra Lopez, Raleigh, NC (US); Jessica Andrea LoDuca Blomberg, Raleigh, NC (US); Melissa Claire Rhodes, Raleigh, NC (US); Jonathan Daniel Heller, San Francisco, CA (US); Amanda Beth Goodman, Wake Forest, NC (US)

Correspondence Address: LOTT & FRIEDLAND, P.A., ONE EAST BROWARD BLVD., SUITE 1609, FORT LAUDERDALE, FL, 33301, US

(73) Assignee: ERIMOS PHARMACEUTICALS LLC, Raleigh, NC (US)

(21) Appl. No.: 11/814,838

(22) PCT Filed: Jan. 27, 2006
(86) PCT No.: PCT/US06/02806

§ 371 Date: Dec. 20, 2007

Related U.S. Application Data
(60) Provisional application No. 60/647,495, filed on Jan. 27, 2005.
(60) Provisional application No. 60/647,648, filed on Jan. 27, 2005.

(51) Int. Cl. A61K 9/14; A61K 31/24

(52) U.S. Cl. 424/486; 424/488; 514/536

(57) ABSTRACT

The present invention provides for compositions, kits and methods for treatment of diseases, where the compositions contain catecholic butanes, including NDGA compounds, such as NDGA derivatives, for example tetra-O-methyl NDGA. The present invention also provides for solubilizing agents and excipients that are suitable for administration of the present compounds into animals via an oral route, whether in a liquid, semi-solid or solid form.

### CROSS-REFERENCE TO RELATED APPLICATIONS

This application claims the benefit of U.S. Provisional Patent Application Nos. 60/647,495 and 60/647,648, both filed Jan. 27, 2005, which applications are incorporated herein by reference in their entireties. This application is also related to an International Patent Application filed simultaneously with the present application and identified as Attorney Docket No. 682714-10WO, entitled “Formulations For Injection Of Catecholic Butanes, Including NDGA Compounds, Into Animals,” the disclosure of which is hereby incorporated herein by reference in its entirety.

### BACKGROUND OF THE INVENTION

This application relates to compositions and methods for administration of catecholic butanes, such as NDGA derivatives, to animals, such as humans, for treatment of diseases, for example, cancer or other proliferative or inflammatory diseases, metabolic diseases or neurodegenerative diseases.

Catecholic butanes such as nordihydroguairetic acid (“NDGA”) have been tested for therapeutic applications in experimental animals by intratumor injection or topical application of such compounds. For example, Jordan et al. in U.S. Pat. No. 5,008,294 described the effect of NDGA on human mammary carcinoma, MX-1, which was implanted subcutaneously into mice on day 0 (Example 2). Mice containing tumors were injected with various doses of NDGA on day 1, in a single intratumor injection. In another experiment, Jordan et al. injected a human breast adenocarcinoma, MS-1, intradermally into mice and treated the animals by topical application of NDGA after day 23 (Example 7).

Huang et al. described the testing of certain derivatives of NDGA (i.e., “NDGA derivatives”) in U.S. Pat. No. 6,214,874. In one experiment, mice were injected with an immortal mouse cell line, C3 cells, and treated by intratumor injection with M₄N alone or in combination with G₄N, both being derivatives of NDGA.

It would be desirable if an oral formulation of these anti-cancer compounds were discovered which would allow for easier administration, especially self-administration without the need for hospitalization. The present invention provides these desirable benefits.

### BRIEF SUMMARY OF THE INVENTION

It is, therefore, one of the objects of the present invention to provide one or more novel oral formulations of catecholic butanes and/or NDGA compounds, such as NDGA derivatives, for treatment of diseases by oral administration of such formulations to subjects in need of such treatment.

It is another one of the objects to provide formulations as above that are safe and have few adverse side effects when administered to animals, including humans.

It is another one of the objects to provide formulations as one or more of the foregoing that have a commercially reasonable period of stability.

It is another one of the objects to provide for formulations as one or more of the foregoing that have a commercially reasonable half-life in circulation upon administration to animals.

In accordance to the foregoing one or more objects of the invention, there is provided embodiments of the present invention as exemplified below:

A composition for oral administration to an animal comprising an active pharmaceutical ingredient and a pharmaceutically acceptable carrier, wherein the active pharmaceutical ingredient comprises a catecholic butane, and the carrier comprises at least one of a solubilizing agent and an excipient selected from the group consisting of: (a) a water-soluble organic solvent other; provided that when the water-soluble organic solvent is propylene glycol, the propylene glycol is in the absence of white petrolatum, in the absence of xanthan gum (also known as xantham gum and xanthum gum) and in the absence of at least one of glycerine or glycine, when the water-soluble organic solvent is polyethylene glycol, the polyethylene glycol is present in the absence of ascorbic acid or butylated hydroxytoluene (“BHT”), and when the polyethylene glycol is polyethylene glycol 400, the polyethylene glycol 400 is present in the absence of polyethylene glycol 8000; (b) a cyclodextrin; (c) an ionic, non-ionic or amphipathic surfactant, provided that when the surfactant is a non-ionic surfactant, the non-ionic surfactant is present in the absence of xanthan gum; (d) a modified cellulose; (e) a water-insoluble lipid, provided that when the water-insoluble lipid is castor oil, the castor oil is present in the absence of beeswax or carnuba wax; and a combination of any of the carriers (a)-(e).

A method of treatment of a disease in a subject comprising: (a) providing the composition of the invention; and (b) administering the composition orally to the subject, wherein the composition comprises an effective amount of the active pharmaceutical ingredient.

A kit for treatment of a disease comprising the composition of the present invention and instructions for use thereof.

### BRIEF DESCRIPTION OF THE DRAWINGS

The foregoing summary, as well as the following detailed description of the invention, will be better understood when read in conjunction with the appended drawings. For the purpose of illustrating the invention, there are shown in the drawings embodiments which are presently preferred. It should be understood, however, that the invention is not limited to the precise arrangements and instrumentalities shown.

In the drawings:

FIG. 1 comprises FIGS. 1A and 1B and depicts the results of cell proliferation assays conducted for the C-33A cell line and the HeLa cell line after M₄N treatment. FIG. 1A is a graphical representation of the ratio of number of cells present after M₄N treatment over the number of cells present in the absence of M₄N treatment, where M₄N was provided in amounts varying from 0 μM to 80 μM in a DMSO formulation. FIG. 1B is a graphical representation of the ratio of number of cells present after M₄N treatment over the number of cells present in the absence of M₄N treatment, where M₄N was provided in amounts varying from 0 μM to 80 μM in a HP-β-CD/PEG formulation (“CPE” formulation).

FIG. 2 comprises FIGS. 2A and 2B and is a graphical representation of cell death measurements based on percentage of dead cells for C-33A cells and HeLa cells in the absence or presence of varying concentrations of M₄N in a DMSO (FIG. 2A) formulation or in a HP-β-CD/PEG formulation (FIG. 2B). The M₄N concentrations varied from 0 μM to 80 μM.

FIG. 3 is a is a histogram showing the absorption of M₄N in Sprague Dawley rats at time points 0.5 hr, 1 hr, 2 hr and 3 hr after oral administration of a single 500 mg/kg dose of M₄N in different liquid solubilizing agents and/or excipients (“carrier”). M₄N was present at a concentration of about 60 mg/mL in all the carriers. The carriers include: (a) HP-β-CD (500 mg/mL)+HPMC (5 mg/mL); (b) HP-β-CD (500 mg/mL)+CMC (5 mg/mL); (c) TPGS (200 mg/mL); (d) TPGS (100 mg/mL)+PEG 400 (50% v/v); (e) Tween® 20; (f) PEG 400 (50% v/v)+Tween® 20 (50% v/v); (g) PEG 400 (33% v/v)+Tween® 20 (33% v/v)+Peppermint oil (33%); (h) Peppermint oil (50%)+PEG 400 (50% v/v); (h) Peppermint oil (50%)+Tween® 20 (50% v/v); (i) Peppermint oil (50%)+Sesame Oil (50%).

FIG. 4 is a graphical representation of the absorption of M₄N by beagle dogs at time points 0.5 hr, 1 hr, 1.5 hr, 2 hr, 4 hr, 6 hr and 8 hr after oral administration of a single 100 mg/kg dose of M₄N in a powder form or in different solid carriers, as follows: (a) M₄N powder; (b) Lyophilized HP-β-CD (81%)+M₄N (185 mg/g); (c) TPGS (20%)+M₄N (133 mg/g); (d) soybean oil (95%)+beeswax (5%)+M₄N (200 mg/g); and (d) olive oil (95%)+beeswax (5%)+M₄N (200 mg/g).

FIG. 5 comprises FIGS. 5A and 5B and is a graphical representation of absorption of non-micronized M₄N in glycerol monooleate by beagle dogs at time points 0.5 hr, 1 hr, 1.5 hr, 2 hr, 4 hr, 8 hr, 12 hr, 24 hr and 36 hr after oral administration of a single 100 mg/kg dose. FIG. 5A is on a non-logarithmic scale. FIG. 5B is on a logarithmic scale.

FIG. 6 comprises FIGS. 6A and 6B and is a graphical representation of absorption of micronized M₄N in glycerol monooleate by beagle dogs at time points 0.5 hr, 1 hr, 1.5 hr, 2 hr, 4 hr, 8 hr, 12 hr, 24 hr and 36 hr after oral administration of a single 100 mg/kg dose. FIG. 6A is on a non-logarithmic scale. FIG. 6B is on a logarithmic scale.

FIG. 7 comprises FIGS. 7A and 7B and is a graphical representation of serum levels of M₄N in various carriers at various concentrations by male beagle dogs at time points 0.5 hr, 1 hr, 1.5 hr, 2 hr, 4 hr, 8 hr, 12 hr, 16 hr, 24 hr and 36 hr after oral administration of a single 75 mg/kg oral dose. The abbreviations of the carrier used, concentration of M₄N administered, and an indication of whether the administration was to dogs that fasted or were fed are best shown in FIG. 7B. FIG. 7A presents the data on a non-logarithmic scale. FIG. 7B presents the data on a logarithmic scale.

FIG. 8 comprises FIGS. 8A and 8B and is a graphical representation of serum levels of M₄N in various carriers at various concentrations by female beagle dogs at time points 0.5 hr, 1 hr, 1.5 hr, 2 hr, 4 hr, 8 hr, 12 hr, 16 hr, 24 hr and 36 hr after oral administration of a single 75 mg/kg oral dose. The abbreviations of the carrier used, concentration of M₄N administered, and an indication of whether the administration was to dogs that fasted or were fed are best shown in FIG. 8B. FIG. 5A presents the data on a non-logarithmic scale. FIG. 8B presents the data on a logarithmic scale.

### DETAILED DESCRIPTION OF THE PREFERRED EMBODIMENTS

The present invention provides for novel compositions, kits and methods for treatment of diseases, including proliferative diseases such as cancer and psoriasis, hypertension, obesity, diabetes, type I or type II, central nervous system diseases or neurodegenerative disorders including, without limitation, pain, Alzheimer's disease, stroke, and inflammatory disease, premalignant neoplasia or dysplasia, infection including viral infections such as HIV, HTLV, HPV, HSV, HBV, EBV, Varicella-zoster, adenovirus, parvovirus, JC virus or others. The present invention provides for novel compositions, kits and methods for treatment of diseases, including proliferative diseases such as cancer and psoriasis, hypertension, obesity, type I or type II diabetes, central nervous system diseases or neurodegenerative diseases including, without limitation, pain, Alzheimer's disease, amyotrophic lateral sclerosis, Parkinson's disease, dementia, stroke, and inflammatory disease, premalignant neoplasia or dysplasia, infection including viral infections such as human immunodeficiency viruses (“HIV”), human T-cell lymphotrophic virus (“HTLV”), human papilloma virus (“HPV”), herpes simplex viruses (“HSV”), hepatitis B virus (“HBV”), Epstein-Barr virus (“EBV”), Varicella-zoster, adenovirus, parvovirus, Jakob Creutzfeldt virus (“JC virus”) or others.

The present invention provides for novel compositions containing the catecholic butanes including NDGA compounds, such as NDGA derivatives, for example M₄N, that are dissolved in certain pharmaceutically acceptable solubilizing agents, which together with other diluents, excipients and the like (collectively, “carrier”) constitute formulations appropriate for administration to subjects, such as humans, for treatment of diseases. Such formulations are suitable for oral administration. A suitable pharmaceutically acceptable carrier includes at least one selected from: (a) a water-soluble organic solvent other than DMSO, such as polyethylene glycol (“PEG”), for example, PEG 300, PEG 400 or PEG 400 monolaurate, propylene glycol (“PG”), polyvinyl pyrrolidone (“PVP”), ethanol, benzyl alcohol or dimethylacetamide; (b) a cyclodextrin or modified cyclodextrin such as hydroxypropyl-β-cyclodextrin (“HP-β-CD”) or sulfobutyl ether β-cyclodextrin (“SBE-β-CD”); (c) an ionic, non-ionic or amphipathic surfactant, such as polyoxyethylene sorbitan monolaurate (also known as polysorbate), which is a non-ionic surfactant, for example, polysorbate 20 and polysorbate 80, commercially available as Tween® 20 or Tween® 80, d-alpha-tocopheryl polyethylene glycol 1000 succinate (“TPGS”), glycerol monooleate (also known as glyceryl monooleate), an esterified fatty acid or a reaction product between ethylene oxide and castor oil in a molar ratio of 35:1, commercially available as Cremophor® EL; (d) a modified cellulose, such as ethyl cellulose (“EC”), hydroxylpropyl methylcellulose (“HPMC”), methyl cellulose (“MC”) or carboxy methylcellulose (“CMC”); and (e) a water-insoluble lipid, such as a wax, oil or a fat emulsion, for example Intralipid®. Preferably, when PG is used, it is used in the absence of white petrolatum, in the absence of xanthan gum, and in the absence of at least one of glycerine or glycine. Preferably, when PEG is used, it is used in the absence of ascorbic acid or BHT; when a non-ionic surfactant is used, it is used in the absence of xanthan gum; and when the oil is castor oil, it is in the absence of beeswax or carnuba wax.

In one embodiment of the invention, the compounds herein are dissolved or dissolved and diluted in different carriers to form an oral liquid composition for administration into animals. For example, in one aspect of the embodiment, the present active pharmaceutical ingredient (“API”) compounds are dissolved in a water-soluble organic solvent such as a PEG 300, PEG 400 or a PEG 400 monolaurate (the “PEG compounds”) or in PG. In another embodiment, the compounds herein are dissolved in a modified cyclodextrin, such as HP-β-CD or SBE-β-CD. In yet another embodiment, the present compounds are solubilized and/or diluted in a combination formulation containing a PEG compound and HP-β-CD. In a further embodiment, the compounds herein are dissolved in a modified cellulose such as HPMC, CMC or EC. In yet another embodiment, the compounds herein are dissolved in another combination formulation containing both a modified cyclodextrin and modified cellulose, such as, for example, HP-β-CD and HPMC or HP-β-CD and CMC.

In yet another embodiment, the compounds herein are dissolved in ionic, non-ionic or amphipathic surfactants such as Tween® 20, Tween® 80, TPGS or an esterified fatty acid. For example, the present compounds can be dissolved in TPGS alone, or Tween® 20 alone, or in combinations such as TPGS and PEG 400, or Tween® 20 and PEG 400.

In a further embodiment, the present compounds are dissolved in a water-insoluble lipid such as a wax, fat emulsion, for example Intralipid®, or oil. For example, the present compounds can be dissolved in peppermint oil alone, or in combinations of peppermint oil with Tween® 20 and PEG 400, or peppermint oil with PEG 400, or peppermint oil with Tween® 20, or peppermint oil with sesame oil.

Of course, ethyl cellulose may be substituted or added in place of the HPMC or CMC in the foregoing examples; PEG 300 or PEG 400 monolaurate can be substituted or added in place of PEG 400 in the foregoing examples; Tween® 80 may be substituted or added in place of Tween® 20 in the foregoing examples; and other oils such as corn oil, olive oil, soybean oil, mineral oil or glycerol, may be substituted or added in place of the peppermint oil or sesame oil in the foregoing examples.

Further, heating may be applied, for example, heating to a temperature of about 30° C. to about 90° C., in the course of formulating any of these compositions to achieve dissolution of the compounds herein or to obtain an evenly distributed suspension of the present compounds.

In still a further embodiment, the present API compounds are administered orally as solids either without any accompanying carrier or with the use of carriers. In one embodiment, the compounds herein are first dissolved in a liquid carrier as in the foregoing examples, and subsequently made into a solid composition for administration as an oral composition. For example, the present compounds are dissolved in a modified cyclodextrin such as HP-β-CD, and the composition is lyophilized to yield a powder that is suitable for oral administration.

In a further embodiment, the present compounds are dissolved or suspended in a TPGS solution, with heating as appropriate to obtain an evenly distributed solution or suspension. Upon cooling, the composition becomes creamy and is suitable for oral administration.

In yet another embodiment, the present compounds are dissolved in oil and beeswax is added to produce a waxy solid composition.

In as yet a further embodiment of the invention, the compounds herein are solubilized in modified celluloses such as EC. The modified celluloses such as ethyl cellulose can be diluted in ethanol (“EtOH”) prior to use.

The present invention also provides water-insoluble lipids as solubilizers for the present compounds. The water-insoluble lipids include, for example, oils as well as mixed fat emulsion compositions such as Intralipid® (Pharmacia & Upjohn, now Pfizer), used as per the manufacturer's recommendation. For example, adult dosage is recommended to be not exceeding 2 g of fat/kg body weight/day (20 mL, 10 mL and 6.7 mL/kg of Intralipid® 10%, 20% and 30%, respectively). Intralipid® 10% is believed to contain in 1,000 mL: purified soybean oil 100 g, purified egg phospholipids 12 g, glycerol anhydrous 22 g, water for injection q.s. ad 1,000 mL. pH is adjusted with sodium hydroxide to pH approximately 8. Intralipid® 20% contains in 1,000 mL: purified soybean oil 200 g, purified egg phospholipids 12 g, glycerol anhydrous 22 g, water for injection q.s. ad 1,000 mL. pH is adjusted with sodium hydroxide to pH approximately 8. Intralipid® 30% contains in 1,000 mL: purified soybean oil 300 g, purified egg phospholipids 12 g, glycerol anhydrous 16.7 g, water for injection q.s. ad 1,000 mL. pH is adjusted with sodium hydroxide to pH approximately 7.5. These Intralipid® products are stored at controlled room temperature below 25° C. and should not be frozen.

In general, in preparing the oral formulations, the compounds herein are first solubilized before other excipients are added so as to produce compositions of higher stability. Unstable formulations are not desirable. Unstable liquid formulations frequently form crystalline precipitates or biphasic solutions. Unstable solid formulations frequently appear grainy and clumpy and sometimes contain runny liquids. An optimal solid formulation appears smooth, homogenous, and has a small melting temperature range. In general, the proportions of excipients in the formulation may influence stability. For example, too little stiffening agent such as beeswax may leave the formulation too runny.

Hence, in general, for the liquid formulations of the present invention, the excipients used should be good solvents of the API or compounds herein, such as M₄N, for example. In other words, the excipients should be able to dissolve the API without heating. The excipients should also be compatible with each other independent of the API such that they can form a stable solution, suspension or emulsion. Also, in general, for the solid formulations of the present invention, the excipients used should also be good solvents of the API to avoid clumps and non-uniform formulations. To avoid solid formulations that are too runny or heterogeneous in texture, which are undesirable, the excipients should be compatible with each other such that they form a smooth homogeneous solid, even in the absence of the API.

The present invention also provides for “placebo” formulations which parallel the formulations containing the API and yet do not contain the API. These placebo formulations are useful for determining compatibility of the different components or excipients in a formulation.

The terms used herein have their ordinary dictionary meaning and as used by persons skilled in the art unless otherwise provided. In particular, the present invention can be better understood in light of the following definitions, which are used with other terms as defined elsewhere herein:

The term “about” as used herein in reference to a concentration or dose means the specified number up to ±10% to 20%.

The term “active pharmaceutical ingredient,” “API” or reference to the “compounds” herein means one or more of the catecholic butanes of Formula I or the NDGA compounds, such as NDGA derivatives, present in the pharmaceutical compositions herein.

“Alkylene dioxy” as used herein refers to methylene or substituted methylene dioxy or ethylene or substituted ethylene dioxy.

“Unsubstituted or substituted amino acid reside or salt thereof” in reference to one of the —R groups in Formula I or Formula II as used herein means an amino acid or a substituted amino acid with one less “H” at its C-terminus by virtue of its linkage to an aromatic group in Formula I or Formula II, where the amino acid or substituted amino acid includes but is not limited to: alanine, arginine, asparagine, aspartate, cysteine, glutamate, glutamine, glycine, histidine, isoleucine, leucine, lysine, methionine, phenylalanine, proline, serine, threonine, tryptophan, tyrosine, valine, 5-hydroxylysine, 4-hydroxyproline, thyroxine, 3-methylhistidine, ε-N-methyllysine, ε-N,N,N-trimethyllysine, aminoadipic acid, γ-carboxyglutamic acid, phosphoserine, phosphothreonine, phosphotyrosine, N-methylarginine, N-acetyllysine, and an N,N-dimethyl-substituted amino acid, or a salt of any of the foregoing, such as a chloride salt.

The “buffer” suitable for use herein includes any buffer conventional in the art, such as, for example, Tris, phosphate, imidazole, and bicarbonate.

A “carrier” as used herein refers to a non-toxic solid, semisolid or liquid filler, diluent, vehicle, excipient, solubilizing agent, encapsulating material or formulation auxiliary of any conventional type, and encompasses all of the components of the composition other than the active pharmaceutical ingredient. The carrier may contain additional agents such as wetting or emulsifying agents, or pH buffering agents. Other materials such as anti-oxidants, humectants, viscosity stabilizers, and similar agents may be added as necessary.

“Catecholic butane” as used herein means a compound of Formula I:

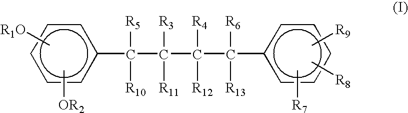

wherein R₁ and R₂ each independently represents —H, a lower alkyl, a lower acyl, an alkylene; or —R₁O and —R₂O each independently represents an unsubstituted or substituted amino acid residue or salt thereof, R₃, R₄, R₅, R₆, R₁₀, R₁₁, R₁₂ and R₁₃ each independently represents —H or a lower alkyl; and R₇, R₈, and R₉ each independently represents —H, —OH, a lower alkoxy, a lower acyloxy, an unsubstituted or substituted amino acid residue or a salt thereof, or any two adjacent groups together may be an alkylene dioxy.

A “cyclodextrin” as used herein means an unmodified cyclodextrin or a modified cyclodextrin, and includes with out limitation α-cyclodextrin, β-cyclodextrin, γ-cyclodextrin and any modified cyclodextrins containing modifications thereto, such as HP-β-CD or SBE-β-CD. Cyclodextrin typically has 6 (α-cyclodextrin), 7 (β-cyclodextrin), and 8 (γ-cyclodextrin) sugars, up to three substitutions per sugar, and 0 to 24 primary substitutions are therefore possible (primary substitutions are defined as substitutions connected directly to the cyclodextrin ring). The modified or unmodified cyclodextrins used in the present invention may have any appropriate number and location of primary substitutions or other modifications.

A “derivative” of NDGA as used herein means an “NDGA derivative” (see below).

The term “disease” as used herein includes all diseases, conditions, infections, syndromes or disorders for which the application of the present composition produces a therapeutic effect. Such “disease” includes, for example without limitation, cancer, psoriasis and other proliferative diseases, inflammatory disorders including rheumatoid arthritis, osteoarthritis, ulcerative colitis, Crohn's disease, atherosclerosis, chronic obstructive pulmonary disease (“COPD”), hypertension, obesity, diabetes, pain, stroke and/or other neuronal disorders or neurodegenerative diseases or conditions, including Alzheimer's disease, Parkinson's disease, multiple sclerosis, amyotrophic lateral sclerosis (“ALS”) and premalignant conditions such as intraepithelial neoplasia or dysplasia, and infectious diseases.

“G₄N” or “tetra-N,N-dimethyl glycinyl NDGA” or “tetra dimethylglycinyl NDGA” as used herein is an NDGA derivative of Formula II in which R₁₄, R₁₅, R₁₆ and R₁₇ each independently represents —O(C═O)CH₂N(CH₃)₂ or —O(C═O)CH₂N⁺(CH₃)₂.Cl⁻, in either a solid form or in solution; and R₁₈ and R₁₉ each represents —CH₃.

“Lower acyl” as used herein means C₁-C₆ acyl, preferably, C₁-C₃ acyl.

“Lower alkyl” as used herein means C₁-C₆ alkyl, preferably, C₁-C₃ alkyl.

“M₄N” or “tetra-O-methyl NDGA” as used herein is an NDGA derivative of Formula II in which R₁₄, R₁₅, R₁₆ and R₁₇ each independently represents —OCH₃, and R₁₈ and R₁₉ are each —CH₃.

A “modified cellulose” as used herein means a cellulose that contains one or more modifications to the cellulose molecule and includes, for example EC, HPMC, CMC and MC.

“NDGA” as used herein means nordihydroguairetic acid and has the following formula:

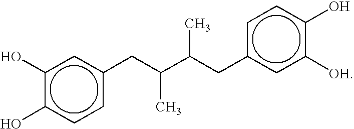

“NDGA compound” as used herein means singly or collectively NDGA and/or any one or more of the NDGA derivatives.

“NDGA derivative” as used herein means a derivative of NDGA having a Formula II

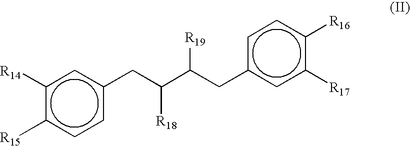

wherein R₁₄, R₁₅, R₁₆ and R₁₇ each independently represents —OH, a lower alkoxy, a lower acyloxy, or an unsubstituted or substituted amino acid residue or pharmaceutically acceptable salt thereof; and R₁₈ and R₁₉ each independently represents —H or a lower alkyl, wherein R₁₄, R₁₅, R₁₆ and R₁₇ are not simultaneously —OH. Thus, the term includes a compound that is a methylated derivative of NDGA, such as tetra-O-methyl NDGA (M₄N), tri-O-methyl NDGA (M₃N), di-O-methyl NDGA (M₂N) and mono-O-methyl NDGA (M₁N). Alternatively, a NDGA derivative may be a compound in which one or more of the hydrogens in the hydroxyl or methyl groups of NDGA are substituted, such as, for example where R₁₄, R₁₅, R₁₆ and R₁₇ each independently represents a lower alkoxy, a lower acyloxy, or an amino acid or substituted amino acid or salt thereof; and R₁₈ and R₁₉ each independently represents —H or an alkyl such as a lower alkyl. The term includes, for example, a compound in which R₁₄, R₁₅, R₁₆ and R₁₇ each independently represents —OCH₃ or —O(C═O)CH₃ or a disubstituted amino acid residue, such as a N,N-dimethyl substituted amino acid residue, such as —O(C═O)CH₂N(CH₃)₂ or —O(C═O)CH₂N⁺(CH₃)₂.Cl⁻; and R₁₈ and R₁₉ each independently represents —H or a lower alkyl, for example, —CH₃ or —CH₂CH₃.

As used herein, “percent,” “percentage” or the symbol “%” means the percent of the component indicated in the composition based on the amount of the carrier present in the composition, on a weight/weight (w/w), weight/volume (w/v) or volume/volume (v/v), as indicated with respect to any particular component, all based on the amount of the carrier present in the composition. Thus, different types of carriers may be present in an amount of up to 100% as indicated, which does not preclude the presence of the API, the amount of which may be indicated as a % or as a certain number of mg present in the composition or a certain number of mg/g present, or a certain number of mg/mL present, where the % or mg/g or mg/mL is based on the amount of the total carrier present in the composition. Certain types of carriers may be present in combination to make up 100% of the carrier.

A “pharmaceutically acceptable carrier” as used herein is nontoxic to recipients at the dosages and concentrations employed, and is compatible with other ingredients of the formulation. For example, the carrier for a formulation containing the present catecholic butane, NDGA compounds or NDGA derivatives preferably does not include oxidizing agents and other compounds that are known to be deleterious to such. A pharmaceutically acceptable carrier comprises a solubilizing agent. Suitable pharmaceutically acceptable carriers include, but are not limited to, water, dextrose, glycerol, saline, ethanol, buffer, Cremaphor® EL, phosphate buffered saline, PEG 300, PEG 400, modified cyclodextrin, and combinations thereof, all as set forth above.

The term “pharmaceutically acceptable excipient,” includes vehicles, adjuvants, or diluents or other auxiliary substances, such as those conventional in the art, which are readily available to the public, and which are non-toxic to recipients at the dosages and concentrations employed, and is compatible with other ingredients of the formulation. For example, pharmaceutically acceptable auxiliary substances include pH adjusting and buffering agents, tonicity adjusting agents, stabilizers, wetting agents and the like.

The term “solubilizing agent” as used herein means a composition in which one or more of the catecholic butanes or NDGA compounds, such as NDGA derivatives, dissolves. A solubilizing agent may also be a carrier or a pharmaceutically acceptable carrier.

The terms “subject,” “host,” and “patient,” are used interchangeably herein to refer to an animal being treated with the present compositions, including, but not limited to, simians, humans, felines, canines, equines, bovines, porcines, ovines, caprines, mammalian farm animals, mammalian sport animals, and mammalian pets.

A “substantially purified” compound in reference to the catecholic butanes or NDGA derivatives as used herein is one that is substantially free of materials that are not the catecholic butane, NDGA compounds or NDGA derivatives (collectively, “non-NDGA materials”). By substantially free is meant at least about 50% free of non-NDGA materials, preferably at least about 70%, more preferably at least about 80%, even more preferably at least about 90%, and still more preferably at least about 95% free of non-NDGA materials.

As used herein, the terms “treatment,” “treating,” and the like, refer to obtaining a desired pharmacologic and/or physiologic effect. The effect may be prophylactic in terms of completely or partially preventing a condition or disease or symptom thereof and/or may be therapeutic in terms of a partial or complete cure for a condition or disease and/or adverse effect attributable to the condition or disease. “Treatment,” of a subject covers, for example, any treatment of a disease in a mammal, particularly in a human, and includes: (a) preventing the disease from occurring in a subject who may be predisposed to the disease but has not yet been diagnosed as having it; (b) inhibiting the disease from developing or progressing, such as, arresting its development; and (c) relieving, alleviating or ameliorating the disease, such as, for example, causing regression or remission of the disease.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

It must be noted that as used herein, the singular forms “a”, “an”, and “the” include plural referents unless the context clearly dictates otherwise. Thus, for example, reference to “a compound” includes a plurality of such compounds and reference to “the NDGA compound” includes reference to one or more NDGA compounds, such as NDGA derivatives, and equivalents thereof known to those skilled in the art.

All publications mentioned herein, including patents, patent applications, and journal articles are incorporated herein by reference in their entireties including the references cited therein, which are also incorporated herein by reference in their entireties.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed. Citations to references mentioned in the text of this application are identified more fully in the bibliography preceding the claims.

The invention described below is given by way of example only and is not to be interpreted in any way as limiting the invention.

Preparation of Catecholic Butanes

The catecholic butanes of the present invention can be prepared by any method known in the art. For example, such compounds can be made as described in U.S. Pat. No. 5,008,294.

Preparation of the NDGA Derivatives

NDGA may be purchased from any available commercial sources, such as, for example, Alexis Biochemicals Corp., San Diego, Calif., U.S.A. (Cat. No. LKT-N5669), or A.G. Scientific, Inc., San Diego, Calif., U.S.A. (Cat. No. N1071), or Cayman Chemical Company, Ann Arbor, Mich., U.S.A. (Cat. No. 70300).

The NDGA derivatives and formulations thereof may made by any process conventional in the art. For example, the NDGA derivatives can be made as described in, U.S. Pat. No. 5,008,294; U.S. Pat. No. 6,291,524; Hwu, J. R. et al. (1998); or McDonald, R. W. et al. (2001).

In one embodiment of the present invention, an NDGA derivative, tetra-O-methyl NDGA, also known as meso-1,4-bis(3,4-dimethoxyphenyl)-2,3-dimethylbutane, or M₄N, is made as follows: a solution is made containing NDGA and potassium hydroxide in methanol in a reaction flask. Dimethyl sulfate is then added to the reaction flask and the reaction is allowed to proceed. The reaction is finally quenched with water, causing the product to precipitate. The precipitate is isolated by filtration and dried in a vacuum oven. The compound is then dissolved in a solution of methylene chloride and toluene and subsequently purified through an alumina column. The solvents are removed by rotary evaporation and the solid is resuspended in isopropanol and isolated by filtration. The filter cake is dried in a vacuum oven. The resulting tetra-O-methyl NDGA (M₄N) is crystallized by refluxing the filter cake in isopropanol and re-isolating the crystals by filtration.

In some embodiments of the present invention, certain NDGA derivatives of the present invention, such as G₄N, also known as meso-1,4-bis[3,4-(dimethylaminoacetoxy)phenyl]-(2R,3S)-dimethylbutane or tetra-dimethylglycinyl NDGA, or a hydrochloride salt thereof, and similar compounds having amino acid substituents, can also be prepared according to conventional methods, as described in, for example, U.S. Pat. No. 6,417,234.

Preparation of the Therapeutic Compositions

The present invention provides compositions, including pharmaceutical compositions, comprising the catecholic butanes, including the NDGA compounds and NDGA derivatives, as active pharmaceutical ingredients (“API”), and pharmaceutically acceptable carriers or excipients. Typically, the compositions of the instant invention will contain from less than about 0.1% up to about 99% of the API, that is, the catecholic butanes, including the NDGA compounds and NDGA derivatives herein; optionally, the present invention will contain about 2% to about 90% of the API.

The present invention additionally provides compositions in which the catecholic butanes, including the NDGA compounds, such as NDGA derivatives, for example M₄N, are present in concentrations of about 1 mg/mL to about 200 mg/mL, or about 10 mg/mL to about 175 mg/mL, or about 20 mg/mL to about 150 mg/mL, or about 30 mg/mL to about 125 mg/mL, or about 40 mg/mL to about 100 mg/mL, or about 50 mg/mL to about 75 mg/mL. In one embodiment, the NDGA compounds are present in the compositions herein at a concentration of about 1 mg/mL, about 2 mg/mL, about 2.5 mg/mL, about 5 mg/mL, about 10 mg/mL, about 15 mg/mL, about 20 mg/mL, about 25 mg/mL, about 30 mg/mL, about 35 mg/mL, about 40 mg/mL, about 45 mg/mL, about 50 mg/mL, about 55 mg/mL, about 60 mg/mL, about 75 mg/mL, about 80 mg/mL, about 90 mg/mL, about 100 mg/mL, about 120 mg/mL, about 125 mg/mL, about 150 mg/mL, about 175 mg/mL or about 200 mg/mL.

The present invention additionally provides compositions in which the catecholic butanes, including the NDGA compounds, such as NDGA derivatives, for example, M₄N, are present in concentrations in the range of about 1 mg/g to about 250 mg/g, or optionally about 20 mg/g to about 200 mg/g, or about 40 mg/g to about 180 mg/g, or about 60 mg/g to about 160 mg/g, or about 80 mg/g to about 130 mg/g, or about 50 mg/g to about 100 mg/g. In one embodiment, the present compounds are present in the compositions herein at a concentration of about 133 mg/g, about 185 mg/g, about 200 mg/g, and about 250 mg/g. Exemplary amounts of the API in the composition include, without limitation, about 20 mg/g, about 50 mg/g, about 75 mg/g, about 100 mg/g, about 120 mg/g, about 130 mg/g, about 140 mg/g, about 150 mg/g, about 175 mg/g or about 200 mg/g.

Expressed alternatively, other embodiments of the composition of the present invention may contain less than about 0.1 mg to about 200 mg or more of the API, such as about 10 mg, about 20 mg, about 25 mg, about 30 mg, about 40 mg, about 50 mg, about 60 mg, about 75 mg, about 100 mg or about 200 mg of the API.

In one embodiment of the invention, there is provided a composition containing an API and a carrier, where the API is a catecholic butane, and the carrier contains a solubilizing agent and/or an excipient for dissolving the API, where the solubilizing agent and/or excipient contains at least one but may contain more of the following: (a) a water-soluble organic solvent, such as PG (propylene glycol) or a PEG (polyethylene glycol) compound, wherein the PEG compound is PEG 300, PEG 400 or PEG 400 monolaurate, for example; (b) a cyclodextrin, such as a modified cyclodextrin; (c) an ionic, non-ionic or amphipathic surfactant, such as Tween® 20, Tween® 80 or TPGS, glycerol monooleate and an esterified fatty acid; (d) a modified cellulose, such as EC, HPMC, MC or CMC; and (e) a water-insoluble lipid such as an oil, wax or a fat emulsion, such as Intralipid®, provided that when the composition contains castor oil, it does not contain beeswax or carnuba wax; and mixtures of the foregoing agents (a)-(e).

In one embodiment, the invention provides a composition as above that is a liquid composition that is suitable for oral administration. In another embodiment, the invention provides a composition as above that is a solid composition that is also suitable for oral administration.

In yet another embodiment, the invention provides a composition as above that does not also contain (a) one of glycerine or glycine; or (b) white petrolatum; or (c) xanthan gum, when the composition contains propylene glycol.

In still another embodiment, the invention provides a composition as above that does not also contain xanthan gum when the composition contains a non-ionic surfactant.

In a further embodiment, the invention provides a composition as above, that does not also contain PEG 8000 or BHT when the composition contains PEG 400.

Among preferred water-soluble organic solvents are ethanol, benzyl alcohol, dimethylacetamide, PVP, PG and PEG compounds such as: PEG 300, PEG 400, or PEG 400 monolaurate. The PEG compound in the present compositions is provided in an amount of about 5% to about 100%, or about 5% to about 60%, or about 10% to about 90%, or about 20% to about 80%, or 30% to about 70%, or about 40% to about 60%, all concentrations being a percentage of volume/volume (v/v). PG may be present at a concentration of about 2.5% to about 100% (v/v).

The concentration of the PEG compounds in the present compositions can vary depending on what other solubilizers or diluents or excipients are also present. For example, the PEG 300, PEG 400 or PEG 400 monolaurate of the present invention can be at a concentration of about 5%, about 10%, about 12.5%, about 15%, about 20%, about 25%, about 30%, about 35%, about 40%, about 45%, about 50%, about 55%, about 60%, about 65%, about 70%, about 75%, about 80%, about 85%, about 90%, or about 95%, all such concentrations being given as a percentage of volume/volume (v/v).

The present invention also provides compositions of catecholic butanes or NDGA compounds in a cyclodextrin, which includes modified cyclodextrins. The cyclodextrins herein may be α-cyclodextrin, β-cyclodextrin, γ-cyclodextrin, and the modified cyclodextrins may include HP-β-CD and SBE-β-CD, for example. In one embodiment, the present composition contains a modified cyclodextrin in a concentration of about 5% to about 80%, or about 10% to about 70%, or about 20% to about 60%, or about 30% to about 50%, all such concentrations being given as a percentage of weight/volume (w/v).

In yet another embodiment, the modified cyclodextrins, such as HP-β-CD, is present in the compositions at a concentration of about 12.5%, about 15%, about 20%, about 25%, about 30%, about 35%, about 40%, about 45%, about 50%, about 55%, about 60%, about 65%, about 70% or about 75%, all such concentrations being given as a percentage of weight/volume (w/v).

Another pharmaceutically acceptable carrier or excipient that may be used alone or with others in the composition of the present invention is an ionic, non-ionic or amphipathic surfactant, such as Cremophor® EL, polysorbates, which are non-ionic surfactants, for example, polysorbate 20 and polysorbate 80, commercially available as Tween® 20 or Tween® 80, TSGS, which is an amphipathic surfactant, among many others. Further examples of suitable surfactants include, without limitation, glycerol monooleate and an esterified fatty acid, such as those typically made by transesterification of vegetable oils, available in several varieties and grades as Labrafil®, Labrasol®, and Gelucire®, from Gattefosse Corp., Paramus, N.J., U.S.A. The surfactant can be present in any desired effective amount, such as at a concentration of about 1% (v/v) to about 100% (v/v), preferably about 9% (v/v) to about 80% (v/v), and more preferably, about 10% (v/v) to about 50% (v/v). As specific examples, preferred concentrations of a non-ionic surfactant are Tween® 20 at a concentration of about 9% (v/v) to about 100% (v/v) and Tween® 80 of about 33% (v/v) to about 100% (v/v). All percentages of the surfactant are volume percentages (v/v).

Another pharmaceutically acceptable carrier or excipient that may be used alone or with others in the composition of the present invention is a modified cellulose, such as EC, HPMC, MC and CMC. The modified cellulose can be present in any desired effective amount, such as a concentration of about 0.1% to about 25%, or about 0.5% to about 7.5%, or about 1.0% to about 5%. As specific examples, EC may be present at a concentration of about 5% to about 20%; HPMC may be present at a concentration of about 0.5% to about 1%; MC may be present at a concentration of about 1% to about 3%; and CMC may be present at a concentration of about 1% to about 4%. The percentages of modified cellulose are in weight per volume (w/v).

In another embodiment, the present invention provides a composition as above that contains a PEG compound, such as PEG 400, for example, and a surfactant, such as Tween® 20, for example, as the solubilizing agent and/or excipient.

Another pharmaceutically acceptable carrier or excipient that may be used alone or with others in the composition of the present invention is a water-insoluble lipid, such as an oil, fat emulsion or wax. The water-insoluble lipid carriers can be present in any desired effective amount, such as a concentration of about 10% to about 100%, or about 15% to about 85%, or about 25% to about 75%.

Non-limiting examples of oils include corn oil, olive oil, peppermint oil, soy bean oil, sesame seed oil, mineral oil and glycerol. In one embodiment, the oil is present at a concentration of about 10% (v/v) to about 100% (v/v). Mixed fat emulsion compositions are available, such as Intralipid® emulsion, as described above. In various embodiments, mixed fat emulsions may be present at a concentration of about 10% (w/v) to about 30% (w/v); and preferably about 20% (w/v). Non-limiting examples of suitable waxes are beeswax and carnuba wax. In one embodiment, the wax is present at a concentration of about 5% (w/w) to about 50% (w/w).

The water-insoluble carriers may be used in combination with any or more of the water-soluble carriers, such as PEG compounds, and the surfactants, such as Tween® 20 or Tween® 80. As a further example, the present composition contains a combination of solubilizing agent and/or excipient such as, for example, oil and surfactant, like peppermint oil and Tween® 20 or oil and a PEG compound, such as peppermint oil and PEG 400.

In a further embodiment, the composition of the invention contains oil, a surfactant and a PEG compound, such as, for example, peppermint oil, Tween® 20, and a PEG 400 compound.

As a further example, the present composition includes a combination of oils or oil and wax, such as, for example, peppermint oil and sesame oil, soybean oil and beeswax or olive oil and beeswax. The beeswax may be present at a concentration in the range of between about 5% to 50% (w/w).

Alternatively, in other embodiments, Tween® 80 may be substituted for Tween® 20, and PEG 300 or PEG 400 monolaurate may be substituted for PEG 400, and any of the oils can be substituted for the peppermint oil, soybean oil, and olive oil.

Still other excipients include glycerol monooleate and various types of esterified fatty acids, such as Labrafil®, Labrasol®, and Gelucire® products. These typically are classified as surfactants or emulsifiers, but the Labrasol®, and Gelucire® products are also used as bioavailability enhancing agents.

Labrafil®, and particularly Labrafil® M 1944 CS, can be used as an excipient with an API, such as M₄N, with the API dissolved in a concentration of about 5 mg/mL to about 500 mg/mL. The final product may be a solution, suspension or solid.

Labrasol® can be used as an excipient with an API, such as M₄N, with the API dissolved in a concentration of about 1 mg/mL to about 500 mg/mL. The final product may be a solution, suspension or solid.

Gelucire®, and particularly Gelucire® 44/14, can be used as an excipient with an API, such as M₄N, with the API dissolved in a concentration of about 30 mg/mL to about 500 mg/mL. The final product is a solid.

Glycerol monooleate can be used as an excipient with an API, such as M₄N, with the API dissolved in a concentration of about 0.1 mg/mL to about 500 mg/mL. The final product can be a solution, suspension or a solid.

Combinations of the various carrier components may be used with the API, as noted above. One non-limiting example of such an embodiment is a composition of 10 mg/ml M₄N in 25% (w/v) PEG 300, 30% (w/v) HP-β-CD, balance of the carrier being “water suitable for injection” into animals (“WFI,” which designates a recognized grade of water in the pharmaceutical industry). In this preferred embodiment, the HP-β-CD has 6 to 8 degrees of substitution, but other substitutions in other embodiments are well within the scope of this invention, as noted above.

Yet other excipients and additives, such as bioavailability enhancing agents, may be used in combination with any of the carriers, in the compositions of the present invention. Suitable bioavailability enhancing agents include, for example without limitation, eugenol, cinnamaldehyde, lecithin, naringenin, naringin and piperin (also known as piperine), in addition to those mentioned above.

It is to be understood that as long as the catecholic butanes herein are dissolved and remain in solution, suspension or maintained within a semi-solid or solid form of the composition, one or more of the solubilizers or diluents or excipients herein may be added to the composition to optimize delivery of such to a subject in need of such treatment.

Other pharmaceutically acceptable carriers or excipients suitable for use herein are described in a variety of publications. Examples of useful carriers or excipients are described in, for example, Gennaro, A. R. (2003); Ansel, H. C. et al. (2004); Rowe, R. C. et al. (2003); and Garg, S. et al. (2001).

The compositions in liquid form may include a buffer, which is selected according to the desired use of the catecholic butanes or NDGA compounds, such as the NDGA derivatives, and may also include other substances appropriate for the intended use. Those skilled in the art can readily select an appropriate buffer, a wide variety of which are known in the art, suitable for an intended use.

Therapeutic Methods

The compositions containing the catecholic butanes, including the NDGA compounds, such as the NDGA derivatives, find use as therapeutic agents or for treatment in subjects in need of such treatment in any number of diseases in which such catecholic butanes or NDGA compounds or derivatives can be used.

The present invention provides for methods and compositions for treatment of disease including, for example, proliferative diseases such as benign and malignant cancer, psoriasis and premalignant conditions and neoplasia, such as intraepithelial neoplasia, or dysplasia. The present invention also provides for treatment of diabetes, including type I and type II diabetes, obesity and complications resulting from such, including cardiovascular diseases, stroke and hypertension. The present invention further provides for treatment of inflammatory diseases including rheumatoid arthritis, osteoarthritis, multiple sclerosis, ulcerative colitis, Crohn's disease, chronic obstructive pulmonary disease (COPD) and other immune system associated diseases. Additionally, the present invention provides for treatment of neurological diseases, including central nervous system diseases and neurodegenerative diseases such as Alzheimer's disease, dementia, amyotrophic lateral sclerosis (ALS) and Parkinson's disease. In a further embodiment, the present invention provides for treatment of infections, such as viral infections including viruses that require Sp1 binding for transcription or replication. Examples of such viruses that require Sp1 binding include: HIV, HTLV, HPV, HSV, HBV, EBV, Varicella-zoster virus, adenovirus, parvovirus and JC virus.

A variety of animal hosts are treatable according to the subject methods, including human and non-human animals. Generally such hosts are “mammals” or “mammalian,” where these terms are used broadly to describe organisms which are within the class mammalia, including the orders carnivore (e.g., dogs and cats), rodentia (e.g., guinea pigs, and rats), and other mammals, including cattle, goats, horses, sheep, rabbits, pigs, and primates (e.g., humans, chimpanzees, and monkeys). In many embodiments, the hosts will be humans. Animal models are of interest for experimental investigations, such as providing a model for treatment of human disease. Further, the present invention is applicable to veterinary care as well.

Formulations, Dosages, and Routes of Administration

In one embodiment of the invention, an effective amount of the present composition is administered to the host, where an “effective amount” means a dosage sufficient to produce a desired result. In some embodiments, for example, the desired result is at least an inhibition of progression of the neoplasia or dysplasia.

The present invention additionally provides compositions in which the active pharmaceutical ingredients such as the catecholic butanes, including the NDGA compounds, such as NDGA derivatives, for example, M₄N, are administered to animals at an oral dose of about less than 1 mg/kg to about 600 mg/kg weight of the animals, such as humans, for example. Optionally, the animals may be treated with 1 mg/kg, or 50 mg/kg, or 100 mg/kg, or 150 mg/kg, or 200 mg/kg, or 250 mg/kg, or 300 mg/kg, or 350 mg/kg, or 400 mg/kg, or 450 mg/kg, or 500 mg/kg, or 550 mg/kg. Such a dose may be administered once or repeatedly over a period of days or weeks or months. Alternatively, such a dose may also be spread out over a period of time, depending on the health of the subject, the subject's sensitivity, the extent of the disease to be treated, the subject's age and the like.

In one embodiment, the therapeutic compositions herein are made by first dissolving the active pharmaceutical ingredient in a solubilizing agent, with stirring and heating as necessary. Other excipients are added to the solubilized mixture to create the desired proportions in terms of texture and stability requirements. In another embodiment, the active pharmaceutical ingredient may not actually dissolve in the solubilizing agent but may simply be evenly distributed in a suspension. In a further embodiment, the solubilized composition may be lyophilized and used in a powder form. The final oral compositions may be in the form of a liquid solution or suspension or may be a solid powder, tablet, or capsule.

As indicated above, the appropriate dose to be administered depends on the subject to be treated, such as the general health of the subject, the age of the subject, the state of the disease or condition, the weight of the subject, the extent of the disease such as the size of the tumor, for example. Generally, about 0.1 mg to about 500 mg or less may be administered to a child and about 0.1 mg to about 5 grams or less may be administered to an adult. The active agent can be administered in a single or, more typically, multiple doses. Preferred dosages for a given agent are readily determinable by those of skill in the art by a variety of means. Other effective dosages can be readily determined by one of ordinary skill in the art through routine trials establishing dose response curves. The amount of agent will, of course, vary depending upon the particular agent used.

The frequency of administration of the active agent, as with the doses, will be determined by the care giver based on age, weight, disease status, health status and patient responsiveness. Thus, the agents may be administered one or more times daily, weekly, monthly or as appropriate as conventionally determined. The agents may be administered intermittently, such as for a period of days, weeks or months, then not again until some time has passed, such as 3 or 6 months, and then administered again for a period of days, weeks, or months.

In pharmaceutical dosage forms, the active agents may be administered alone or in appropriate association, as well as in combination, with other pharmaceutically active agents or therapeutics including other small molecules, antibodies or protein therapeutics.

In addition, if desired, the carrier or excipient may contain minor amounts of auxiliary substances such as pH adjusting and buffering agents, tonicity adjusting agents, stabilizers, wetting agents or emulsifying agents. Actual methods of preparing such dosage forms are known, or will be apparent, to those skilled in the art. See, e.g., Remington's Pharmaceutical Sciences, 20th ed., Mack Publishing Co. Rawlins E A, (1997). The composition or formulation to be administered will, in any event, contain a quantity of the API adequate to achieve the desired state in the subject being treated.

Kits with multiple or unit doses of the active agent are included in the present invention. In such kits, in addition to the containers holding the multiple or unit doses of the compositions containing the catecholic butanes including the NDGA compounds, such as the NDGA derivatives, will be an informational package insert with instructions describing the use and attendant benefits of the drugs in treating pathological condition of interest. Optionally, an applicator for administration of the present composition is included in each kit.

The Examples set forth below are exemplary in nature and are not to be interpreted as limiting the present invention.

### EXAMPLE 1

### Formulations Containing M₄N in HP-β-CD and/or PEG 300

In this example, M₄N was prepared as described in PCT/US2004/016117 and was solubilized in a solubilizing agent. The resulting solution was optionally mixed with an excipient and/or a diluent. The solubilizing agent and the excipient may be used interchangeably or in combination with each other. One solubilizing agent or excipient used was endotoxin-controlled hydroxypropyl-β-cyclodextrin (“HP-β-CD”) obtained from Research Diagnostics, Inc. (Cat. No. RDI-82004HPB, lot no. H3N188P) (Flanders, N.J., U.S.A.). Another solubilizing agent or excipient used was PEG 300, obtained from Spectrum Chemicals, Inc. (Cat. No. P0108, lot no. TB1228) (Gardena, Calif., U.S.A.).

In one embodiment of the invention, HP-β-CD and PEG 300 were present in a single formulation. To make this formulation, M₄N was first dissolved in PEG 300 to form a M₄N in PEG 300 solution (“M₄N/PEG 300”). The M₄N/PEG 300 solution was then added to a pre-made solution of HP-β-CD to form a M₄N solution in a PEG 300 and HP-β-CD (hereafter, a “CPE” formulation).

When preparing the HP-β-CD solution, a volume expansion must be accounted for. For example, for a 40% (w/v) HP-β-CD solution, a volume expansion of 0.7 mL/g (i.e., 0.7 mL of water displaced per gram of HP-β-CD added) must be accounted for.

A 100 mL solution of 40% HP-β-CD for use as a solubilizing agent and/or excipient was made as follows: 65 mL of WFI were placed in a glass beaker containing a stir bar. The beaker was placed on a magnetic plate, and the stir bar was set to stir at medium speed. About forty (40) grams of HP-β-CD were added slowly to the stirring WFI, using a spatula to direct the HP-β-CD to the center of the beaker so as to prevent HP-β-CD crystals from sticking to the beaker wall. The HP-β-CD solution was stirred for about 24 hr or until the HP-β-CD was dissolved completely upon visual inspection. The resulting solution measured about 93 mL. About 7 mL of WFI were added to this resulting solution to obtain 100 mL, producing a final solution of about 40% HP-β-CD. The final solution was stirred for about 1 hr, stored at room temperature, and was protected from light. This method of making the modified cyclodextrin solution may be scaled up or down to obtain the desired volume or concentration. Other concentrations or other modified cyclodextrin solutions may be similarly made, for example, by substituting HP-β-CD with other modified cyclodextrins, adjusted for the appropriate concentrations in the process described above.

A 10 mL solution of M₄N at a concentration of about 10 mg/mL in 40% BP-β-CD was made as follows. About 10 mL of the 40% HP-β-CD solution were added to a glass beaker containing a stir bar. The beaker was placed on a magnetic plate and the stir bar was set to stir at medium speed. About 10 mg of M₄N were slowly added to the 40% HP-β-CD in the center of the beaker with the aid of a spatula. The M₄N/40% HP-β-CD mixture was stirred for 2 hr or until all M₄N was uniformly suspended without any clumps being present. The M₄N/40% HP-β-CD mixture was optionally heated at 80° C. for about 30 min. (or longer if a larger volume of solution was desired, for example, 1 hr at 80° C. for 100 mL of the M₄N/HP-β-CD mixture), or longer as needed to ensure complete dissolution of M₄N. The M₄N/HP-β-CD mixture or solution was observed for presence of any undissolved M₄N by holding the beaker against a white background followed by a dark background, looking for presence of particulates. The final M₄N/40% HP-β-CD solution was stored at room temperature and was kept protected from light. This process may be scaled up or down to obtain the requisite volume or concentration of M₄N. Formulations containing M₄N in other cyclodextrin solutions may be similarly made, such as, for example, by substituting HP-β-CD in the process described above with other cyclodextrins. Results shown in Table 1 demonstrate that M₄N remained in solution after cooling at concentrations of 1 mg/mL and 10 mg/mL in the 40% HP-β-CD formulation for greater than 7 days.

A 100 mL solution of M₄N at a concentration of about 25 mg/mL in PEG 300 was made as follows. About 100 mL of PEG 300 were added to a glass beaker containing a stir bar. The beaker was placed on a magnetic plate and the stir bar was set to stir at medium speed. About 2.5 g of M₄N were slowly added to the PEG 300 in the center of the beaker with the aid of a spatula to prevent M₄N from sticking to the beaker wall. The M₄N/PEG 300 mixture was stirred for 24 hr or until all M₄N had dissolved or was uniformly suspended without any clumps being present. The M₄N/PEG 300 mixture was optionally heated at about 60° C. for about 30 min. (or longer if a larger volume of solution was desired, for example, 1 hr at 60° C. for 500 mL of the M₄N/PEG 300 mixture), or longer as needed to ensure complete dissolution of M₄N. The M₄N/PEG 300 mixture or solution was observed for presence of any undissolved M₄N by holding the beaker against a white background followed by a dark background, looking for presence of particulates. After all M₄N was observed to be dissolved, the resulting M₄N/PEG 300 solution was used immediately or before the expiration of 48 hr, otherwise crystals or other precipitates might form. If crystals formed, the M₄N/PEG 300 solution could be heated again at 60° C. for about 1 hr, with stirring, on a hot magnetic plate until all M₄N was dissolved back in solution. The final M₄N/PEG 300 solution was stored at room temperature and was kept protected from light. This process may be scaled up or down to obtain the requisite volume or concentration of M₄N. Formulations containing M₄N in other PEGs may be similarly made, such as, for example, by substituting PEG 300 in the process described above with PEG 400 or PEG 400 monolaurate.

A 100 mL stock solution of a formulation containing 50% PEG 300 (v/v), 20% HP-β-CD (w/v) and 12.5 mg M₄N was made by adding 50 mL of the 40% pre-made HP-β-CD solution (made as described above) to a glass beaker containing a stir bar on a magnetic plate, with the stir bar stirring at medium speed, and slowly adding 50 mL of the pre-made M₄N/PEG 300 solution (made as described above), for example, at a rate of about 10 mL per min. The M₄N/PEG 300 was added by use of a pipette to the center of the beaker to avoid its sticking to the walls of the beaker and to ensure complete dissolution. The addition of M₄N/PEG 300 to the HP-β-CD solution initially appeared as a white solution, but eventually became clear upon continuous mixing. This recipe may be scaled up or down as appropriate to produce the desired volume or concentration of M₄N/PEG 300 and HP-β-CD. The stock solution was filter-sterilized using a 0.22 μm PVDF membrane, such as a pre-sterilized vacuum driven disposal bottle-top filter membrane obtained from Millipore (Cat. No. SCGV T05 RE) (Billerica, Mass., U.S.A.). The filtration process was driven by vacuum force and the filtrate is collected in pre-sterilized 250 mL glass bottles. The bottles were then sealed tightly, stored at room temperature and protected from light. A stock solution of M₄N/PEG 400 or M₄N/PEG 400 monolaurate in HP-β-CD can be similarly made by substituting PEG 300 with PEG 400 or PEG 400 monolaurate in the processes described above.

The M₄N/PEG 300/HP-β-CD, M₄N/PEG 400/HP-β-CD or M₄N/PEG 400 monolaurate/HP-β-CD) stock solution made in the foregoing manner can be diluted prior to use in vitro or for administration into animals. If dilution is necessary, the stock solution is preferably diluted in WFI, instead of saline, for example, so as to keep the osmolarity down. To make a 100 mL of a 1:1 dilution of the stock solution in WFI, about 50 mL of the stock solution was added to a glass vial. About 50 mL of WFI was added to the 50 mL of the stock solution in the vial to form a diluted solution. The glass vial was closed and the diluted solution was mixed well by shaking and inverting the vial a few times. The diluted solution was filter-sterilized using a 0.22 μm PVDF membrane, such as a pre-sterilized vacuum driven disposal bottle-top filter membrane obtained from Millipore (Cat. No. SCGV T05 RE) (Billerica, Mass., U.S.A.). The filtration process was driven by vacuum force and the filtrate was collected in pre-sterilized 250 mL glass bottles. The bottles were then sealed tightly, stored at room temperature and protected from light. This process may be scaled up or down to obtain the requisite volume or dilutions, such as 1:2 or 1:4 dilutions, for example.

A formulation suitable for use as placebo control containing 50% PEG 300 and 20% HP-β-CD can be made as follows. To make a 100 mL solution of the placebo or control formulation, about 50 mL of 40% HP-β-CD are added to a glass beaker containing a stir bar on a magnetic plate. The magnetic plate is set to stir the HP-β-CD solution at medium speed. About 50 mL of PEG 300 are slowly added to the 50 mL of HP-β-CD in the glass beaker by pipette to the center of the beaker to prevent the PEG 300 from sticking to the wall of the beaker. The mixture is stirred for about 1 hr or until mixing is complete. This placebo formulation is filter-sterilized using a 0.22 μm PVDF membrane filter driven by vacuum force. The filtrate is collected in pre-sterilized 250 mL glass bottles. The glass bottles are sealed tightly, stored at room temperature, and kept protected from light. This recipe may be scaled up or down as required to produce the desired concentration and volume amounts. Further, PEG 300 may be substituted with PEG 400 or PEG 400 monolaurate, as desired.

Results of the solubility of M₄N in formulations containing HP-β-CD and/or PEG 300, PEG 400, and in formulations containing HP-β-CD and propylene glycol (“PG”), hydroxypropyl methylcellulose (HPMC), carboxyl methylcellulose (CMC), polyvinyl pyrrolidone (PVP), or Tween® 80 made in accordance to the foregoing or similar processes as well as characteristics of the resulting formulations are shown in Tables 1-5, where N represents “No” and Y represents “Yes.”

TABLE 1
Modified Cyclodextrins as Solubilizing Agents and/or Excipients

| | Excipient | Drug | | | | |
|---|---|---|---|---|---|---|
| | Concentration (in | Concentration | | Dissolution | Dissolution | |
| | w/v unless | (in mg/mL | Dissolution | After | After | Stability |
| | otherwise | unless otherwise | After | Heating at | Cool | at Room |
| Excipients | specified) | specified) | Rotation | X° C. | Down | Temperature |
| α-CD | 15% | 1 | N | N at 90° | | |
| | | 10 | N | N at 90° | | |
| | | 50 | N | N at 90° | | |
| | | 100 | N | N at 90° | | |
| β-CD | 1.50% | 1 | N | N at 90° | | |
| | | 10 | N | N at 90° | | |
| γ-CD | 5% | 1 | N | N at 90° | | |
| HP-β-CD | 50% | 1 | N | Y at 90° | Y | >7 days |
| | | 10 | N | Y at 90° | Y | >7 days |
| | | 20 | N | N at 90° | | |
| | | 50 | N | N at 90° | | |
| | | 100 | N | N at 90° | | |
| | 40% | 1 | N | Y at 80° | Y | >7 days |
| | | 10 | N | Y at 80° | Y | >7 days |
| | | 12 | N | N at 80° | | |
| | | 14 | N | N at 80° | | |
| | | 16 | N | N at 80° | | |
| | | 20 | N | N at 80° | | |
| | | 50 | N | N at 80° | | |
| | | 100 | N | N at 80° | | |
| | 30% | 1 | N | Y at 90° | Y | >7 days |
| | | 10 | N | Y at 90° | Y | <3 days |
| | | 20 | N | N at 90° | | |
| | | 50 | N | N at 90° | | |
| | | 100 | N | N at 90° | | |
| | 20% | 1 | N | Y at 90° | Y | >7 days |
| | | 10 | N | N at 90° | | |
| | 81.5% (w/w), | 185 mg/g | powder | | | |
| | Lyophilized | | | | | |
| HP-β-CD | 50% in saline | 10 | N | Y at 90° | Y | >7 days |
| | 20% in saline | 1 | N | | | |
| | | 10 | N | | | |
| | | 50 | N | | | |
| HP-β-CD | 40% HP-β-CD, | 1 | N | N at 80° | | |
| and | 2.5% PG (v/v) | | | | | |
| Propylene | | | | | | |
| Glycol | | | | | | |
| (PG) | | | | | | |
| | | 10 | N | N at 80° | | |
| HP-β-CD | 50% HP-β-CD, | 1 | Suspension | | | >7 days |
| and CMC | 0.5% CMC | | | | | |
| | | 20 | Suspension | | | >7 days |
| | | 60 | Suspension | | | >7 days |
| HP-β-CD | 50% HP-β-CD, | 1 | N | Y at 90° | Y | <3 days |
| and PVP | 1.25% PVP (w/v) | | | | | |
| | | 10 | N | N | | |
| | | 50 | N | N | | |
| | 40% HP-β-CD, 1% | 1 | N | N | | |
| | PVP (w/v) | | | | | |
| | | 10 | N | N | | |
| HP-β-CD | 27% HP-β-CD, | 13.3 | N | Y at 60° | Y | >7 days |
| and PEG | 33% PEG 300 (v/v) | | | | | |
| 300 | | | | | | |
| | 23% HP-β-CD, 43 | 12.9 | N | Y at 60° | Y | >7 days |
| | PEG 300 (v/v) | | | | | |
| | 20% HP-β-CD, | 12.5 | N | Y at 60° | Y | >7 days |
| | 50% PEG 300 (v/v) | | | | | |
| | 15% HP-β-CD, | 12.5 | N | Y at 60° | Y | <7 days |
| | 50% PEG 300 (v/v) | | | | | |
| | 12.5% HP-β-CD, | 12.5 | N | Y at 60° | Y | <1 day |
| | 50% PEG 300 (v/v) | | | | | |
| | 13% HP-β-CD, | 16.7 | N | Y at 60° | N | |
| | 67% PEG 300 (v/v) | | | | | |
| | 10% HP-β-CD, | 19.3 | N | Y at 60° | N | |
| | 77% PEG 300 (v/v) | | | | | |
| | 10% HP-β-CD, | 6.25 | N | Y at 60° | Y | >7 days |
| | 25% PEG 300 (v/v) | | | | | |
| | 6.7% HP-β-CD, | 4.17 | N | Y at 60° | Y | >7 days |
| | 16.7% PEG 300 | | | | | |
| | (v/v) | | | | | |
| | 5% HP-β-CD, | 3.13 | N | Y at 60° | Y | <7 days |
| | 12.5% PEG 300 | | | | | |
| | (v/v) | | | | | |
| HP-β-CD | 32% HP-β-CD, | 10 | N | Y at 60° | Y | >7 days |
| and PEG | 20% PEG 400 (v/v) | | | | | |
| 400 | | | | | | |
| | 30% HP-β-CD, | 12.5 | N | Y at 60° | Y | >7 days |
| | 25% PEG 400 (v/v) | | | | | |
| | 27% HP-β-CD, | 13.3 | N | Y at 60° | Y | >7 days |
| | 33% PEG 400 (v/v) | | | | | |
| | 23% HP-β-CD, | 12.9 | N | Y at 60° C. | Y | >7 days |
| | 43% PEG 400 (v/v) | | | | | |
| | 20% HP-β-CD, | 12.5 | N | Y at 60° | Y | <7 days |
| | 50% PEG 400 (v/v) | | | | | |
| | 15% HP-β-CD, | 12.5 | N | Y at 60° | Y | <3 days |
| | 50% PEG 400 (v/v) | | | | | |
| | 12.5% HP-β-CD, | 12.5 | N | Y at 60° | Y | <1 day |
| | 50% PEG 400 (v/v) | | | | | |
| | 40% HP-β-CD, 5% | 10 | N | Y at 60° | N | |
| | PEG 400 (v/v) | | | | | |
| HP-β-CD | 27% HP-β-CD, | 13.3 | N | Y at 60° | N | |
| and | 33% Tween ® 80 | | | | | |
| Tween ® | (v/v) | | | | | |
| 80 | | | | | | |

All formulations withstand 4° C. for 24 hr and 5 min. centrifugation at 5000 rpm without forming visible precipitates. The formulation containing 50% PEG 300, 20% HP-β-CD, 12.5 mg/mL M₄N stock solution withstands 4° C. for at least 4 months. Dilutions of the same stock made in 1:1 or 1:2 dilutions also withstand 4° C. for at least 4 months.

TABLE 2
Formulations of M₄N in PEG 300 and HP-β-CD

| | | | For each 10 mg | | For each 50 mg | | For each 100 mg | |
|---|---|---|---|---|---|---|---|---|
| | | | of M₄N | | of M₄N | | of M₄N | |
| Undiluted Stock Solutions | | | PEG | | PEG | | PEG | |
| PEG | HP-β- | | 300 in | HP-β- | 300 in | HP-β- | 300 in | HP-β- |
| 300 | CD | M₄N | mL | CD | mL | CD | mL | CD |
| (v/v) | (w/v) | (mg/mL) | (mg) | (mg) | (mg) | (mg) | (mg) | (mg) |
| 50% | 15% | 12.5 | 0.4 (450) | 120 | 2.0 (2250) | 600 | 4.0 (4500) | 1200 |
| 50% | 20% | 12.5 | 0.4 (450) | 160 | 2.0 (2250) | 800 | 4.0 (4500) | 1600 |
| 43% | 23% | 12.9 | 0.33 (375) | 178 | 1.67 (1875) | 890 | 3.33 (3746) | 1780 |
| 33% | 27% | 13.3 | 0.25 (281) | 200 | 1.25 (1406) | 1000 | 2.5 (2813) | 2000 |

TABLE 3
Formulations of M₄N in PEG 400 and HP-β-CD

| | | | For each 10 mg | | For each 50 mg | | For each 100 mg | |
|---|---|---|---|---|---|---|---|---|
| | | | of M₄N | | of M₄N | | of M₄N | |
| Undiluted Stock Solutions | | | PEG | | PEG | | PEG | |
| PEG | HP-β- | | 400 in | HP-β- | 400 in | HP-β- | 400 in | HP-β- |
| 400 | CD | M₄N | mL | CD | mL | CD | mL | CD |
| (v/v) | (w/v) | (mg/mL) | (mg) | (mg) | (mg) | (mg) | (mg) | (mg) |
| 50% | 20% | 12.5 | 0.4 (450) | 160 | 2.0 (2250) | 800 | 4.0 (4500) | 1600 |
| 43% | 23% | 12.9 | 0.33 mL (375) | 178 | 1.67 (1875) | 890 | 3.33 (3746) | 1780 |
| 33% | 27% | 13.3 | 0.25 (281) | 200 | 1.25 (1406) | 1000 | 2.5 (2813) | 2000 |
| 25% | 30% | 12.5 | 0.20 (225) | 240 | 1.0 (1125) | 1200 | 2.0 (2250) | 2400 |
| 20% | 32% | 10.0 | 0.20 (225) | 320 | 1.0 (1125) | 1600 | 2.0 (2250) | 3200 |

TABLE 4
Stability of M₄N formulations in PEG 300

| | PEG | HP-β- | | |
|---|---|---|---|---|
| | 300 | CD | M₄N | Stability at Room |
| Formulation | (v/v) | (w/v) | (mg/mL) | Temperature |
| A | 50% | 15% | 12.5 | >3 days |
| B | 50% | 20% | 12.5 | >5 months |
| C | 43% | 23% | 12.9 | >5 months |
| D | 33% | 27% | 13.3 | >5 months |

TABLE 5
Stability of M₄N formulations in PEG 400

| | PEG | HP-β- | | |
|---|---|---|---|---|
| | 400 | CD | M₄N | Stability at Room |
| Formulation | (v/v) | (w/v) | (mg/mL) | Temperature |
| E | 50% | 20% | 12.5 | >6 days |
| F | 43% | 23% | 12.9 | >5 months |
| G | 33% | 27% | 13.3 | >5 months |
| H | 25% | 30% | 12.5 | >5 months |
| I | 20% | 32% | 10.0 | >5 months |

Similarly, M₄N may be solubilized in other solubilizing agents, such as water-soluble organic solvents including ethanol, PVP (polyvinyl pyrrolidone), propylene glycol or glycerol.

### EXAMPLE 2

### Effect of M₄N in DMSO or Combination PEG 300/HP-β-CD Formulation on Proliferation and Death of Tumor Cells in Culture

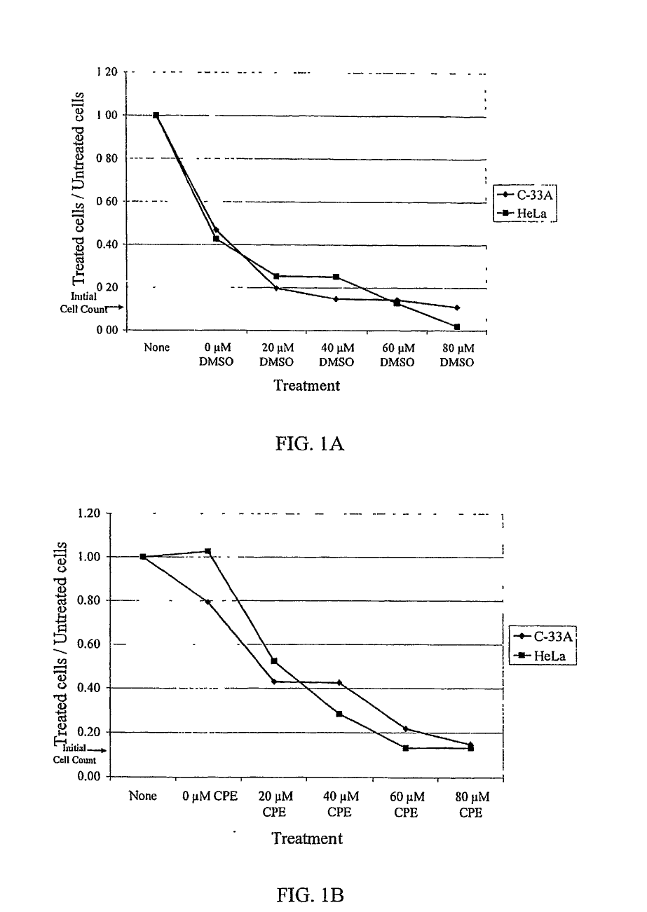

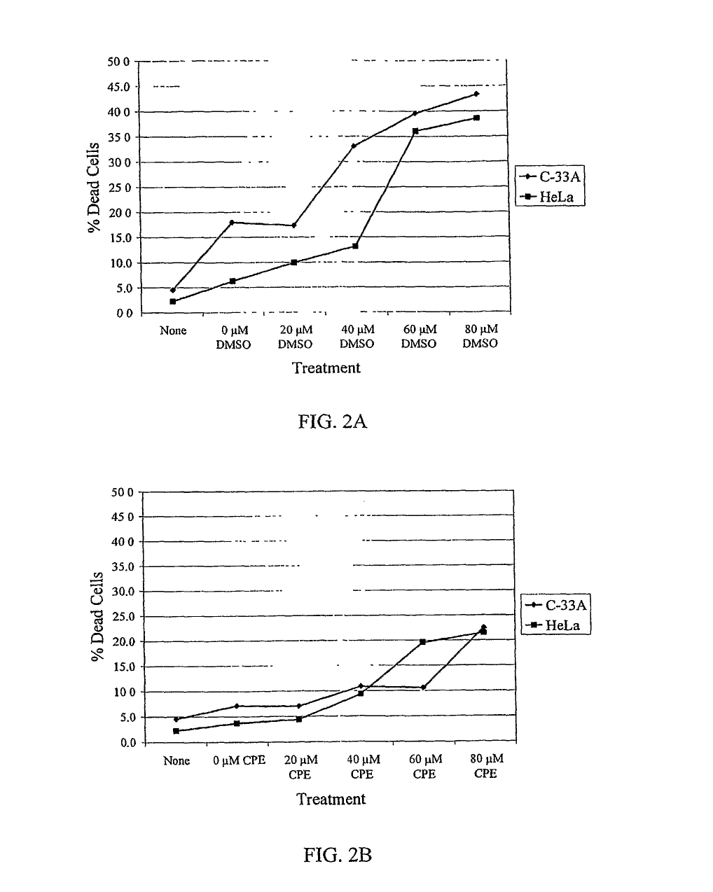

M₄N in 10% (w/v) HP-β-CD and 25% (v/v) PEG 300 (hereafter, the “CPE formulation”), M₄N in 30% (w/v) HP-β-CD and 25% (v/v) PEG 300 (hereafter, the “CPE 25/30 formulation”) and M₄N in 27% (w/v) HP-β-CD and 33% (v/v) PEG 300 (hereafter, the “CPE 33/27 formulation”) was tested for their effects on cell death and proliferation on two different tumor cell lines: HeLa, an HPV-18 positive human cervical cancer cell line, and C-33A, an HPV-negative human cervical cancer cell line. M₄N in DMSO was also tested in parallel. Both tumor cell lines were treated with increasing amounts of M₄N: 0 μM, 20 μM, 40 μM, 60 μM and 80 μM, for 72 hr with the DMSO or the CPE formulation. Each formulation was added to total 1% of the growth media (Minimal Essential Medium with L-glutamine supplemented with 10% fetal bovine serum, 1 mM sodium pyruvate, 1× non-essential amino acid solution, and 1,000 IU/mL penicillin/1,000 μg/mL streptomycin solution). Control cells were grown under the same conditions and were left untreated. After 72 hr of treatment or no treatment, the total number of cells and the number of live cells in each sample were counted, using the trypan blue exclusion method. The cell proliferation rate and the percentage of dead cells in each sample were analyzed. Results of this experiment are shown in FIG. 1, FIG. 2, and Tables 6 through 12.

FIG. 1 is a graphical representation of the ratio of the number of treated cells/number of untreated cells plotted against increasing concentrations of M₄N in either the DMSO formulation or the CPE formulation for treatment of the C-33A cells and HeLa cells. FIG. 2 is a graphical representation of the percentage of dead cells plotted against increasing concentrations of M₄N and in either DMSO formulation or the CPE formulation for treatment of the two cancer cell lines, C-33A and HeLa cells in culture.

Results show that DMSO alone, in the absence of M₄N, has a significant anti-proliferative effect and some toxic effect, as measured by % of dead cells, on both of the tumor cell lines tested as compared to the untreated controls. In contrast, the CPE formulation alone has an anti-proliferative effect and very little toxic effect on the two tumor cell lines when compared to the untreated controls.

Cell proliferation rate was reduced in both cell lines after treatment with M₄N in either the DMSO formulation or the CPE formulation, when compared to the non-M₄N-treated controls (i.e., 0 μg/mL or 0 μM of M₄N) in the same formulation. In fact, the anti-proliferative effect appeared to be M₄N dose-dependent in the CPE formulation. In the CPE formulation, for example, about 20 μM or 7.2 μg/mL of M₄N was found to be sufficient to cause about a 50% inhibition in cell proliferation for both tumor cell types. Further increases in the concentration of M₄N in the CPE formulation resulted in further increases in anti-proliferative effect for both cell types.

In general, higher doses M₄N in either the DMSO formulation or the CPE formulation induced higher percentages of cell death for both the C-33A cells and the HeLa cells. However, M₄N in the DMSO formulation was more toxic to cells than the corresponding concentrations of M₄N in the CPE formulation. While the highest concentration of M₄N tested (80 μM or 28.7 μg/mL) in DMSO formulation promoted cell death in about 40% of the cell population, the same concentration of M₄N in the CPE formulation promoted cell death in only about 20% of the cell population in this experiment.

These results were found to be reproducible in both cell lines. Data from this study indicate that M₄N in the CPE formulation has the ability to arrest cell proliferation, similar to M₄N in the DMSO formulation, while inducing less cellular toxicity than the DMSO formulation. Data were collected from continuing time points to test the effectiveness of the CPE formulation over time. The data showed that after a twelve month period of being stored at 2-8° C. the CPE formulation is just as effective as when it is new. The viability of cells remained similar over the twelve months the CPE formulation was stored. Cell death and proliferation rates remained within the same range.

Data were collected to compare the original CPE formulation with the new CPE 25/30 formulation. Studies were conducted at 0 and 3 months to test the effectiveness of the formulation over time as well as to test how well the new formulation works in relation to the old. The data showed that the CPE 25/30 formulation is as effective in inhibiting the growth of tumor cells as is the original CPE formulation, Cell viability was similar between HeLa cells treated with various concentrations of drug using either the CPE formulation or the CPE 25/30 formulation. Cell death and proliferation remained within the same range even over time.

Information was collected comparing the original CPE formulation with the CPE 33/27 formulation at time zero on HeLa cells. The data showed that CPE 33/27 had the same affects on HeLa cells in cell viability, percent of dead cells and proliferation rate.

TABLE 6
Effect of M₄N in DMSO or the CPE Formulation on C-33A Cells

| Treatment | % Viability | % Dead Cells | Proliferation Rate |
|---|---|---|---|
| None | 95.4 | 4.6 | 1.00 |
| 0 μM DMSO | 82.0 | 18.0 | 0.47 |
| 20 μM DMSO | 82.6 | 17.4 | 0.20 |
| 40 μM DMSO | 67.0 | 33.0 | 0.15 |
| 60 μM DMSO | 60.4 | 39.6 | 0.14 |
| 80 μM DMSO | 56.7 | 43.3 | 0.11 |
| 0 μM CPE | 92.8 | 7.2 | 0.79 |
| 20 μM CPE | 93.0 | 7.0 | 0.43 |
| 40 μM CPE | 89.1 | 10.9 | 0.43 |
| 60 μM CPE | 89.4 | 10.6 | 0.22 |
| 80 μM CPE | 77.5 | 22.5 | 0.15 |

TABLE 7
Effect of M₄N in DMSO or the CPE Formulation
on HeLa Cells at time 0

| Treatment | % Viability | % Dead Cells | Proliferation Rate |
|---|---|---|---|
| None | 97.7 | 2.3 | 1.00 |
| 0 μM DMSO | 93.7 | 6.3 | 0.43 |
| 20 μM DMSO | 90.1 | 9.9 | 0.25 |
| 40 μM DMSO | 86.8 | 13.2 | 0.25 |
| 60 μM DMSO | 63.9 | 36.1 | 0.13 |
| 80 μM DMSO | 61.4 | 38.6 | 0.02 |
| 0 μM CPE | 96.3 | 3.7 | 1.03 |
| 20 μM CPE | 95.6 | 4.4 | 0.52 |
| 40 μM CPE | 90.6 | 9.4 | 0.28 |
| 60 μM CPE | 80.4 | 19.6 | 0.13 |
| 80 μM CPE | 78.5 | 21.5 | 0.13 |

TABLE 8
Effect of M₄N in DMSO or the CPE formulation on HeLa Cells
at 9 months

| Treatment | % Viability | % Dead Cells | Proliferation Rate |
|---|---|---|---|
| None | 95.7 | 4.2 | 1.00 |
| 0 μM DMSO | 94.9 | 5.1 | 0.76 |
| 20 μM DMSO | 89.3 | 10.7 | 0.19 |
| 40 μM DMSO | 91.2 | 8.8 | 0.14 |
| 60 μM DMSO | 67.5 | 32.5 | 0.03 |
| 80 μM DMSO | 50.0 | 50.0 | 0.02 |
| 0 μM CPE | 95.6 | 4.4 | 0.72 |
| 20 μM CPE | 68.7 | 31.3 | 0.24 |
| 40 μM CPE | 72.8 | 27.2 | 0.10 |
| 60 μM CPE | 86.4 | 13.6 | 0.09 |
| 80 μM CPE | 88.1 | 11.9 | 0.08 |

TABLE 9
Effect of M₄N in DMSO or the CPE formulation on HeLa Cells
at 12 months

| Treatment | % Viability | % Dead Cells | Proliferation Rate |
|---|---|---|---|
| None | 92.1 | 7.9 | 1.00 |
| 0 μM DMSO | 90.5 | 9.5 | 0.77 |
| 20 μM DMSO | 87.4 | 12.6 | 0.23 |
| 40 μM DMSO | 86.4 | 13.7 | 0.04 |
| 60 μM DMSO | 64.6 | 35.4 | 0.04 |
| 80 μM DMSO | 76.5 | 23.6 | 0.15 |
| 0 μM CPE | 95.6 | 4.4 | 1.50 |
| 20 μM CPE | 96.8 | 3.3 | 0.74 |
| 40 μM CPE | 95.0 | 5.0 | 0.21 |
| 60 μM CPE | 75.0 | 25.0 | 0.05 |
| 80 μM CPE | 52.8 | 47.2 | 0.03 |

TABLE 10
Comparison of the CPE formulation and the CPE 25/30 Formulation
in HeLa Cells

| Treatment | % Viability | % Dead Cells | Proliferation Rate |
|---|---|---|---|
| None | 93.5 | 6.6 | 1.00 |
| 0 μM CPE | 92.3 | 7.8 | 0.92 |
| 20 μM CPE | 93.5 | 6.6 | 0.33 |
| 40 μM CPE | 76.7 | 23.4 | 0.08 |
| 60 μM CPE | 44.5 | 55.6 | 0.03 |
| 80 μM CPE | 43.4 | 56.7 | 0.04 |
| 0 μM CPE 25/30 | 90.7 | 9.3 | 0.81 |
| 20 μM CPE 25/30 | 90.7 | 9.4 | 0.34 |
| 40 μM CPE 25/30 | 89.1 | 11.0 | 0.31 |
| 60 μM CPE 25/30 | 77.3 | 22.8 | 0.12 |
| 80 μM CPE 25/30 | 54.8 | 45.2 | 0.07 |

TABLE 11
Comparison of the CPE formulation and the CPE 25/30 Formulation
in HeLa Cells at 3 months

| Treatment | % Viability | % Dead Cells | Proliferation Rate |
|---|---|---|---|
| None | 95.8 | 4.2 | 1.00 |
| 0 μM CPE | 95.0 | 5.0 | 0.91 |
| 20 μM CPE | 91.0 | 9.0 | 0.39 |
| 40 μM CPE | 83.5 | 16.5 | 0.09 |
| 60 μM CPE | 56.9 | 43.1 | 0.03 |
| 80 μM CPE | 71.0 | 29.0 | 0.03 |
| 0 μM CPE 25/30 | 93.4 | 6.6 | 0.87 |
| 20 μM CPE 25/30 | 88.1 | 11.9 | 0.39 |
| 40 μM CPE 25/30 | 86.6 | 13.4 | 0.32 |
| 60 μM CPE 25/30 | 76.9 | 23.1 | 0.11 |
| 80 μM CPE 25/30 | 64.4 | 35.6 | 0.04 |

TABLE 12
Comparison of CPE formulation and the CPE 33/27 formulation
in HeLa cells at time zero

| Treatment | % Viability | % Dead Cells | Proliferation Rate |
|---|---|---|---|
| None | 95.0 | 5.0 | 1.00 |
| 0 μM CPE | 96.4 | 3.6 | 1.11 |
| 20 μM CPE | 88.1 | 11.9 | 0.35 |
| 40 μM CPE | 74.2 | 25.9 | 0.14 |
| 60 μM CPE | 73.9 | 26.2 | 0.10 |
| 80 μM CPE | 78.4 | 21.6 | 0.11 |
| 0 μM CPE 33/27 | 95.4 | 4.7 | 0.92 |
| 20 μM CPE 33/27 | 89.2 | 10.9 | 0.43 |
| 40 μM CPE 33/27 | 86.4 | 13.6 | 0.19 |
| 60 μM CPE 33/27 | 70.7 | 29.3 | 0.10 |
| 80 μM CPE 33/27 | 75.0 | 25.0 | 0.09 |

### EXAMPLE 3

### Multiple Lots of M₄N can be Used and Create the Same Results

Various lots of M₄N were tested to show the effectiveness of the drug of different lots. HeLa cells were treated with increasing amounts of M₄N: 0 μM, 20 μM, 40 μM, 60 μM and 80 μM, for 72 hr with the CPE formulation. Each formulation was added to total 1% of the growth media (Minimal Essential Medium with L-glutamine supplemented with 10% fetal bovine serum, 1 mM sodium pyruvate, 1× non-essential amino acid solution, and 1,000 IU/mL penicillin/1,000 μg/mL streptomycin solution). Control cells were grown under the same conditions and were left untreated. After 72 hr of treatment or no treatment, the total number of cells and the number of live cells in each sample were counted, using the trypan blue exclusion method. The cell proliferation rate and the percentage of dead cells in each sample were analyzed. Results of this experiment are shown in Tables 13 and 14. These result show that regardless of the lot of M₄N used, the effectiveness of the drug remains the same.

TABLE 13
Treatment of HeLa cells with various lots of M₄N (lot EM1001)

| Treatment | % Viability | % Dead Cells | Proliferation Rate |
|---|---|---|---|
| None | 96.2 | 3.7 | 1.00 |
| 0 μM CPE | 96.2 | 3.8 | 0.92 |
| 20 μM CPE | 94.9 | 5.1 | 0.25 |
| 40 μM CPE | 73.7 | 26.3 | 0.10 |
| 60 μM CPE | 44.4 | 55.6 | 0.01 |
| 80 μM CPE | 59.8 | 40.2 | 0.01 |

TABLE 14
Treatment of HeLa cells with various lots of M₄N (lot EM1002)

| Treatment | % Viability | % Dead Cells | Proliferation Rate |
|---|---|---|---|
| None | 95.9 | 4.1 | 1.00 |
| 0 μM CPE | 96.1 | 3.8 | 0.65 |
| 20 μM CPE | 88.0 | 12.0 | 0.32 |
| 40 μM CPE | 74.2 | 25.8 | 0.11 |
| 60 μM CPE | 41.9 | 58.1 | 0.05 |
| 80 μM CPE | 40.3 | 59.7 | 0.03 |

### EXAMPLE 4

### Solubility of M₄N in Modified Celluloses

A 10 mL solution of 50% HP-β-CD (w/v) and 0.5% hydroxypropyl methylcellulose (“HPMC”) (w/v) for use as a solubilizing agent and/or excipient was made as follows: 5.9 mL of WFI were placed in a glass beaker containing a stir bar. The beaker was placed on a magnetic plate, and the stir bar was set to stir at medium speed. Five grams of HP-β-CD were added slowly to the stirring WFI, using a spatula to direct the HP-β-CD to the center of the beaker. The HP-β-CD solution was stirred for about 24 hr or until the HP-β-CD was dissolved completely upon visual inspection. The resulting solution measured about 9.4 mL. About 0.6 mL of WFI was added to this resulting solution to reach 10 mL, to produce a solution of 50% HP-β-CD (w/v). Fifty milligrams of HPMC were added to the 50% HP-β-CD solution and stirred for about 1 hr or until the HPMC was dissolved upon visual inspection. The final solution was stirred for about 1 hr and was then stored at room temperature, protected from light. This method of making modified cyclodextrins with modified celluloses may be scaled up or down to obtain the desired volume or concentration of HP-β-CD/HPMC solution. Other modified cyclodextrin/modified cellulose solutions may be similarly made, for example, by substituting HP-β-CD with other modified cyclodextrins, or HPMC with other modified celluloses, in the process described above.

A 10 mL solution of M₄N at a concentration of about 10 mg/mL in 50% HP-β-CD/0.5% HPMC was made as follows. About 10 mL of the 50% HP-β-CD/0.5% HPMC solution were added to a glass beaker containing a stir bar. The beaker was placed on a magnetic plate and the stir bar was set to stir at medium speed. About 100 mg of M₄N were slowly added to the 50% HP-β-CD/0.5% HPMC in the center of the beaker with the aid of a spatula. The M₄N/50% HP-β-CD/0.5% HPMC mixture was stirred for 24 hr or until all M₄N was uniformly suspended without any clumps being present. The M₄N/50% HP-β-CD/0.5% HPMC mixture was heated at about 90° C. for about 30 min. (or longer if a larger volume of solution was desired, for example, 1 hr at 90° C. for 500 mL of the M₄N/50% HP-β-CD/0.5% HPMC mixture), or longer as needed to ensure complete dissolution of M₄N. The M₄N/50% HP-β-CD/0.5% HPMC mixture was observed for presence of any undissolved M₄N by holding the beaker against a white background followed by a dark background, looking for presence of particulates. The final M₄N/50% HP-β-CD/0.5% HPMC solution was stored at room temperature and was kept protected from light. M₄N was dissolved in this 50% HP-β-CD/0.5% HPMC formulation at the 1 mg/mL and 10 mg/mL concentrations when heated at 90° C. and remained in solution after cool down, with stability at room temperature for greater than 7 days. M₄N did not dissolve in this same formulation at the 50 mg/mL concentration even at 90° C.

The foregoing process may be scaled up or down to obtain the requisite volume or concentration of M₄N. Formulations containing M₄N in other cyclodextrin/cellulose solutions may be similarly made, such as, for example, by substituting HP-β-CD in the process described above with other cyclodextrins, or by substituting HPMC with other modified celluloses.

A 10 mL solution of 5% ethylcellulose (“EC”) in ethanol (w/v) for use as a solubilizing agent and/or excipient was made as follows: 10 mL of 100% ethyl alcohol (“EtOH”) were placed in a glass beaker containing a stir bar, covered by a round Teflon® cover. The beaker was placed on a magnetic plate, and the stir bar was set to stir at medium speed. Five hundred (500) milligrams of EC was added slowly to the stirring ethanol, using a spatula to direct the EC to the center of the beaker so as to prevent EC powder from sticking to the beaker wall. The EC solution was stirred for about 2 hr or until the EC was dissolved completely upon visual inspection. The final solution was stored at room temperature, and was protected from light.

This method of making modified cellulose solutions may be scaled up or down to obtain the desired volume or concentration. Other modified cellulose solutions may be similarly made, for example, by substituting EC with other modified celluloses, in the process described above.

A 10 mL solution of M₄N at a concentration of about 20 mg/mL in 5% EC (w/v) (the “EC formulation”) was made as follows. About 10 mL of 5% EC formulation, made as described above, were added to a glass beaker containing a stir bar. The beaker was placed on a magnetic plate and the stir bar was set to stir at medium speed, and covered with a round Teflon® cover. About 200 mg of M₄N were slowly added to the 5% EC formulation in the center of the beaker with the aid of a spatula to prevent M₄N from sticking to the beaker wall. The M₄N/EC mixture was stirred for 2 hr or until all M₄N had dissolved or was uniformly suspended without any clumps being present. The M₄N/EC mixture was heated at about 60° C. for about 30 min. (or longer if a larger volume of solution was desired, for example, 1 hr at 60° C. for 500 mL of the M₄N/EC mixture), or longer as needed to ensure complete dissolution of M₄N. The M₄N/EC mixture or solution was observed for presence of any undissolved M₄N by holding the beaker against a white background followed by a dark background, looking for presence of particulates. The final M₄N/EC solution was stored at room temperature and was kept protected from light.

The foregoing process may be scaled up or down to obtain the requisite volume or concentration of M₄N and the heating temperature may be increased or decreased to achieve dissolution of M₄N. Formulations containing M₄N in other modified celluloses may be similarly made, such as, for example, HPMC, MC, and CMC. Results of the solubility of M₄N in the modified celluloses are set forth in Table 15.

Results show that M₄N was not soluble in 2.3% (w/v) HPMC at any of the concentrations tested or upon heating to 90° C. M₄N formed a suspension in 1% (w/v) HPMC at the 10 mg/mL concentration. This suspension was stable at room temperature for less than 2 days.

In the presence of HP-β-CD (50% w/v) and HPMC (0.5% w/v), M₄N was solubilized at 90° C. and remained in solution upon cooling at the 1 mg/mL and 10 mg/mL concentrations. These solutions were stable at room temperature for more than 7 days. M₄N did not dissolve in the same HP-β-CD (50% w/v) and HPMC (0.5% w/v) composition at the 50 mg/mL concentration even upon heating to 90° C.

In another experiment, M₄N formed suspensions in the absence of heat in the HP-β-CD (50% w/v) and HPMC (0.5% w/v) composition at all concentrations tested, i.e., 1 mg/mL, 10 mg/mL, 20 mg/mL and 50 mg/mL. These suspensions were stable at room temperature for more than 7 days.

Results in Table 15 also show that M₄N was soluble at 1 mg/mL in the EC formulation without application of heat, the solution being stable at room temperature for greater than 3 days. M₄N was soluble at the 10 mg/mL concentration at 40° C. and remained in solution after cooling, this solution being stable at room temperature for greater than 3 days. M₄N was soluble in the EC formulation at the 20 mg/mL concentration at 60° C. and remained in solution after cooling, this solution being stable at room temperature for greater than 3 days. M₄N at the 30 mg/mL concentration was soluble in the EC formulation at 60° C., but did not remain in solution upon cooling. Higher concentrations of M₄N, such as at the 50 mg/mL or 100 mg/mL levels, were soluble in the EC formulation at 90° C., but did not remain in solution upon cooling.

M₄N also formed suspensions in 1% low viscosity CMC at the 10 mg/mL and 20 mg/mL levels. These suspensions were stable at room temperature for less than 2 days.

TABLE 15
Solubility of M₄N in Formulations containing modified celluloses

| | | Drug | | | | |
|---|---|---|---|---|---|---|
| | Excipient | Concentration | | | | |
| | Concentration | (in | | | | |
| | (in w/v | mg/mL | | | Dissolution | Stability |
| | unless | unless | Dissolution | | After | Time at |
| | otherwise | otherwise | After | Dissolution | Cool- | Room |
| Excipients | stated) | state) | Rotation | After Heating | Down | Temperature |
| HPMC | 2.3% | 1 | N | N at 90° C. | | |
| | | 10 | N | N at 90° C. | | |
| | | 50 | N | N at 90° C. | | |
| | | 100 | N | N at 90° C. | | |
| | 1% | 10 | suspension | | | <2 days |
| HP-β-CD | 50% HP-β- | 1 | N | Y at 90° C. | Y | >7 days |
| and HPMC | CD, 0.5% | | | | | |
| | HPMC | | | | | |
| | | 10 | N | Y at 90° C. | Y | >7 days |
| | | 50 | N | N at 90° C. | | |
| | 50% HP-β- | 1 | Suspension | | | >7 days |
| | CD, 0.5% | | | | | |
| | HPMC | | | | | |
| | | 10 | Suspension | | | >7 days |
| | | 20 | Suspension | | | >7 days |
| | | 50 | Suspension | | | >7 days |
| | 84% HP-β- | 150 mg/g | powder | | | |
| | CD, 1% | | | | | |
| | HPMC, | | | | | |
| | Lyophilized | | | | | |
| EC | 5% in EtOH | 1 | Y | | | >3 days |
| | | 10 | N | Y at 40° C. | Y | >3 days |
| | | 20 | N | Y at 60° C. | Y | >3 days |
| | | 30 | N | Y at 60° C. | N | |
| | | 50 | N | Y at 90° C. | N | |
| | | 100 | N | Y at 90° C. | N | |
| MC | 2% | 1 | N | N at 90° C. | | |
| (Low | | 10 | N | N at 90° C. | | |
| viscosity) | | | | | | |
| CMC | 1% | 1 | N | N at 90° C. | | |
| (High | | 10 | N | N at 90° C. | | |
| Viscosity) | | | | | | |
| CMC | 4% | 1 | N | N at 90° C. | | |
| (Low | | 10 | N | N at 90° C. | | |
| Viscosity) | | | | | | |
| | 1% | 10 | suspension | | | <2 days |
| | | 20 | suspension | | | <2 days |

### EXAMPLE 5

### Solubility of M₄N in Water-Insoluble Lipids and in Water-Soluble Organic Solvents

A 10 mL solution of M₄N at a concentration of about 50 mg/mL in sesame oil was made as follows. About 10 mL of sesame oil were added to a glass beaker containing a stir bar. The beaker was placed on a magnetic plate and the stir bar was set to stir at medium speed. About 500 mg of M₄N were slowly added to the sesame oil in the center of the beaker with the aid of a spatula to prevent M₄N from sticking to the beaker wall. The M₄N/sesame oil mixture was stirred for about 2 hr or until all M₄N had dissolved or was uniformly suspended without any clump being present. The M₄N/sesame oil mixture was heated at about 60° C. for about 30 min. (or longer if a larger volume of solution was desired, for example, 1 hr at 60° C. for 500 mL of the M₄N/sesame oil mixture), or longer as need to ensure complete dissolution of M₄N. The M₄N/sesame oil mixture or solution was observed for presence of any undissolved M₄N by holding the beaker against a white background followed by a dark background, looking for presence of particulates. If crystals formed, the M₄N/sesame oil solution could be heated again at 60° C. for about 1 hr, with stirring, on a hot magnetic plate until all M₄N was dissolved back in solution. The final M₄N/sesame oil solution was stored at room temperature and was kept protected from light.

The foregoing process may be scaled up or down to obtain the requisite volume or concentration of M₄N and the heating temperature may be increased or decreased to achieve dissolution of M₄N. Formulations containing M₄N in other water-insoluble lipids may be similarly made, such as, for example, by substituting sesame oil in the process described above with corn oil, olive oil, soybean oil, peppermint oil, or other solubilizing agents, and combinations thereof. Results are shown in Table 16.

A 10 g mixture of M₄N at a concentration of about 200 mg/g in 95% olive oil and 5% beeswax was made as follows. About 7.6 mL of olive oil were added to a glass beaker containing a stir bar. The beaker was placed on a magnetic plate and the stir bar was set to stir at medium speed. About 2 g of M₄N were slowly added to the olive oil in the center of the beaker with the aid of a spatula to prevent M₄N from sticking to the beaker wall. The M₄N/olive oil mixture was stirred for about 2 hr or until all M₄N had dissolved or was uniformly suspended without any clumps being present. The M₄N/olive oil mixture was optionally heated at about 60° C. for about 30 min. (or longer if a larger volume of solution was desired, for example, 1 hr at 60° C. for 500 mL of the M₄N/olive oil mixture), or longer as need to ensure complete dissolution of M₄N. The M₄N/olive oil mixture was observed for presence of any undissolved M₄N by holding the beaker against a white background followed by a dark background, looking for presence of particulates. If crystals formed, the M₄N/olive oil solution could be heated again at 60° C. for about 1 hr, with stirring, on a hot magnetic plate until all M₄N was dissolved back in solution. About 400 mg of white beeswax were added to a glass beaker containing a stir bar. The beaker was also placed on a magnetic plate and the stir bar was set to stir at medium speed. The beeswax was heated at about 50° C. for about 30 min. (or longer if a larger quantity was desired), or until all the beeswax was melted. The M₄N/olive oil solution was then added to about 400 mg of melted beeswax and stirred and heated at about 50° C. for about 30 min or until all the M₄N/olive oil/beeswax mixture had dissolved or was uniformly mixed. The stir bar was removed from the beaker and the M₄N/olive oil/beeswax mixture was allowed to cool down. The final M₄N/olive oil/beeswax mixture was stored at room temperature and was kept protected from light.

This process may be scaled up or down to obtain the requisite volume or concentration of M₄N. Formulations containing M₄N in other water-insoluble lipids may be similarly made, such as, for example, by substituting olive oil in the process described above with corn oil, sesame oil, soybean oil, peppermint oil, lecithin, or other solubilizing agents, and combinations thereof, and substituting beeswax with paraffin, PEG 3350, or other stiffening agents, and combinations thereof. Also, if desired, in combination with any of the carriers, a bioavailability enhancing agent may be included, such as eugenol, cinnamaldehyde, lecithin, naringenin, naringin and piperin (also known as piperine), for example. Results are shown in Table 16.

A 10 mL solution of M₄N at a concentration of about 60 mg/mL in 85% sesame oil and 15% Tween® 20 was made as follows. About 8.5 mL of sesame oil were added to a glass beaker containing a stir bar. The beaker was placed on a magnetic plate and the stir bar was set to stir at medium speed. About 1.5 mL of Tween® 20 was slowly added to the center of the beaker. About 600 mg of M₄N were slowly added to the sesame oil/Tween® 20 mixture in the center of the beaker with the aid of a spatula to prevent M₄N from sticking to the beaker wall. The M₄N/sesame oil/Tween® 20 mixture was stirred for about 2 hr or until all M₄N had dissolved or was uniformly suspended without any clumps being present. The M₄N/sesame oil/Tween®120 mixture was heated at about 60° C. for about 30 min. (or longer if a larger volume of solution was desired, for example, 1 hr at 60° C. for 500 mL of the M₄N/sesame oil/Tween® 20 mixture), or longer as need to ensure complete dissolution of M₄N. The M₄N/sesame oil/Tween® 20 mixture was observed for presence of any undissolved M₄N by holding the beaker against a white background followed by a dark background, looking for presence of particulates. The final M₄N/sesame oil/Tween® 20 solution was stored at room temperature and was kept protected from light. If crystals were to form during storage, the M₄N/sesame oil/Tween® 20 solution could be heated again at 60° C., with stirring, on a hot magnetic plate until all M₄N dissolved back in solution.

This process may be scaled up or down to obtain the requisite volume or concentration of M₄N and the heating temperature may be increased or decreased to achieve dissolution of M₄N. Formulations containing M₄N in other water-insoluble lipids combined with non-ionic surfactants, ionic surfactants or water-soluble organic solvents may be similarly made, such as, for example, by substituting sesame oil or Tween® 20 in the process described above with corn oil, olive oil, soybean oil, peppermint oil, Tween® 80, TPGS, lecithin, PEG 300, PEG 400, PEG 400 monolaurate, glycerol, PVP, PG, or other solubilizing agents, and combinations thereof. Results of the solubility of M₄N in water-insoluble lipids are set forth in Table 16.

Formulations containing M₄N in other water-insoluble lipids combined with non-ionic, ionic or amphipathic surfactants or water-soluble organic solvents may be similarly made, such as, for example, by substituting sesame oil in the process described above with corn oil, olive oil, soybean oil, peppermint oil, or mineral oil and substituting Tween® 20 with Tween® 80, TPGS, lecithin, PEG 300, PEG 400, PEG 400 monolaurate, glycerol, PVP, PG, or other solubilizing agents, and combinations thereof. Results of the solubility of M₄N in water-insoluble lipids and the stability of such compositions are set forth in Table 16. Stability in reference to the clear liquid solutions herein refers to the time it takes to form crystallized precipitation in solution. A stable solution is one that is clear and free of particulates for an extended period of time.

Table 16 shows, for example, that M₄N was soluble in corn oil when heated at 60° C. at concentrations up to 100 mg/mL and, except at the 100 mg/mL level, M₄N at lower concentrations remained in solution after cooling, the solutions being stable for greater than 3 days at the 1 and 10 mg/mL concentrations, for less than 3 days at the 20, 40 and 50 mg/mL levels and for less than 1 day at the 60 mg/mL level. Further, M₄N was soluble in olive oil at 60° C. at the 30 mg/mL level but did not remain in solution after cooling.

In sesame oil, M₄N was soluble at room temperature at the 10 mg/mL level. At 60° C., M₄N was soluble up to the 50 mg/mL concentration, and remained in solution after cooling. The 10 mg/mL and 20 mg/mL solutions were stable at room temperature for more than 3 days, the 30 mg/mL solution was stable at room temperature for less than 3 days, and the 50 mg/mL solution was stable at room temperature for less than 1 day.

In peppermint oil, M₄N was soluble between 1 mg/mL and 125 mg/mL. At concentrations of up to 20 mg/mL, M₄N was soluble without heating. These compositions were stable at room temperature for more than 3 days. M₄N was soluble at higher concentrations, up to the 125 mg/mL level, upon heating at 40° C. and remained in solution after cooling. Of these higher concentrations of M₄N in peppermint oil, the 40 mg/mL composition was stable at room temperature for more than 3 days.

M₄N also solubilized in soybean oil at concentrations of up to 50 mg/mL upon heating at 60° C. Of these, only the 10 mg/mL concentration remained in solution upon cooling, and this solution remained stable for more than 7 days.

M₄N was also soluble in mineral oil when heated at 60° C. at concentrations of up to 200 mg/mL. The 10 mg/mL and 50 mg/mL compositions remained in solution upon cooling. Of these, the 10 mg/mL solution was stable for more than 7 days at room temperature.

In a combination of 50% peppermint oil and 50% PEG 300, M₄N was soluble up to 125 mg/mL when heated at 35° C. Between the 40 mg/mL and 60 mg/mL levels, M₄N remained in solution after cooling and was stable at room temperature for more than 7 days. In a combination of 60% peppermint oil and 40% PEG 300, M₄N was soluble at 60 mg/mL upon heating at 40° C. and remained in solution upon cooling. This composition was stable at room temperature for more than 3 days.

When peppermint oil was combined with PEG 400 at 50% each, M₄N was solubilized up to 125 mg/mL when heated at 40° C. At 40 mg/mL and 60 mg/mL M₄N concentrations, the compound remained in solution upon cooling and the compositions were stable at room temperature for more than 7 days. In a combination of 60% peppermint oil and 40% PEG 400, M₄N was solubilized at 60 mg/mL upon heating at 40° C.

In a combination of 50% peppermint oil and 50% Tween® 20, M₄N was soluble up to 125 mg/mL tested upon heating at 40° C. Of these, M₄N remained in solution after cooling at the 40 mg/mL and 60 mg/mL concentrations.

In another combination, such as peppermint oil and sesame oil, each 50%, M₄N was soluble up to the 60 mg/mL concentration tested. M₄N was soluble at 20 mg/mL at room temperature, the composition being stable at room temperature for more than 3 days. The 40 mg/mL and 60 mg/mL solutions of M₄N remained in solution upon cooling, and were stable for more than 3 days at room temperature.

In yet another combination, containing 33% peppermint oil, 33% Tween® 20 and 33% PEG 400, M₄N was soluble at 60 mg/mL when heated at 40° C., and remained in solution upon cooling. These compositions were stable for more than 3 days.

Table 16 also shows, for example, that M₄N can be solubilized in soybean oil and made into a waxy solid when combined with beeswax. In addition, M₄N can also be solubilized in olive oil and beeswax can be added to turn the composition into a waxy solid.

TABLE 16
Solubility of M₄N in water-insoluble Lipids

| | | Drug | | | | | |
|---|---|---|---|---|---|---|---|
| | Excipient | Concentration | | | | | |
| | Concentration | (in | | | | | |
| | (in w/v | mg/mL | | | | Dissolution | Stability |
| | unless | unless | | Dissolution | Dissolution | After | Time at |
| | otherwise | otherwise | | After | After | Cool- | Room |
| Excipients | stated) | stated) | | Rotation | Heating | Down | Temperature |
| Corn Oil | 100% | 1 | | N | Y at 60° C. | Y | >3 days |
| | | 10 | | N | Y at 60° C. | Y | >3 days |
| | | 20 | | N | Y at 60° C. | Y | <3 days |
| | | 40 | | N | Y at 60° C. | Y | <3 days |
| | | 50 | | N | Y at 60° C. | Y | <3 days |
| | | 60 | | N | Y at 60° C. | Y | <1 day |
| | | 100 | | N | Y at 60° C. | N | |
| Olive Oil | 100% | 30 | | N | Y at 60° C. | N | |
| Sesame Oil | 100% | 10 | | Y | | | >3 days |
| | | 20 | | N | Y at 60° C. | Y | >3 days |
| | | 30 | | N | Y at 60° C. | Y | <3 days |
| | | 50 | | N | Y at 60° C. | Y | <1 day |
| Peppermint | 100% | 1 | | Y | | | >3 days |
| Oil | | | | | | | |
| | | 10 | | Y | | | >3 days |
| | | 20 | | Y | | | >3 days |
| | | 40 | | N | Y at 40° C. | Y | >3 days |
| | | 60 | | N | Y at 40° C. | Y | <3 days |
| | | 100 | | N | Y at 40° C. | Y | <1 day |
| | | 125 | | N | Y at 40° C. | Y | <1 day |
| Soybean Oil | 100% | 10 | | N | Y at 60° C. | Y | >7 days |
| | | 30 | | N | Y at 60° C. | N | |
| | | 50 | | N | Y at 60° C. | N | |
| Mineral Oil | 100% | 10 | | N | Y at 60° C. | Y | >7 days |
| | | 50 | | N | Y at 60° C. | Y | <1 day |
| | | 100 | | N | Y at 60° C. | N | |
| | | 200 | | N | Y at 60° C. | N | |
| Olive Oil, | 80% Olive | 60 | | N | Y at 70° C. | N | |
| Soybean Oil | Oil, 20% | | | | | | |
| | Soybean Oil | | | | | | |
| Sesame Oil, | 75% Sesame | 24.3 | | N | Y at 60° C. | Y | <7 days |
| Tween ® 20 | Oil, 9% | | | | | | |
| and Glycerol | Tween ® 20, | | | | | | |
| | 16% Glycerol | | | | | | |
| | 10% Oil | 2.4 | | Y | | | <1 day |
| | emulsion in | | | | | | |
| | 90% saline | | | | | | |
| Sesame Oil | 89% Sesame | 29 | | N | Y at 60° C. | Y | >7 days |
| and Tween ® | Oil, 11% | | | | | | |
| 20 | Tween ® 20 | | | | | | |
| | 10% Oil | 2.9 | | Y | | | <3 days |
| | emulsion in | | | | | | |
| | 90% saline | | | | | | |
| | 85% Sesame | 40 | | N | Y at 45° C. | N | |
| | Oil, 15% | | | | | | |
| | Tween ® 20 | | | | | | |
| | 85% Sesame | 60 | | N | Y at 55° C. | N | |
| | Oil, 15% | | | | | | |
| | Tween ® 20 | | | | | | |
| Peppermint | 50% | 40 | | N | Y at 35° C. | Y | >7 days |
| Oil, PEG 300 | Peppermint | | | | | | |
| | Oil, 50% PEG | | | | | | |
| | 300 | | | | | | |
| | | 60 | | N | Y at 35° C. | Y | >7 days |
| | | 125 | | N | Y at 35° C. | N | |
| | 60% | 60 | | N | Y at 40° C. | Y | >3 days |
| | Peppermint | | | | | | |
| | Oil, 40% PEG | | | | | | |
| | 300 | | | | | | |
| Peppermint | 50% | 40 | | N | Y at 40° C. | Y | >7 days |
| Oil, PEG 400 | Peppermint | | | | | | |
| | Oil, 50% PEG | | | | | | |
| | 400 | | | | | | |
| | | 60 | | N | Y at 40° C. | Y | >7 days |
| | | 100 | | N | Y at 40° C. | N | |
| | | 125 | | N | Y at 40° C. | N | |
| | 60% | 60 | | N | Y at 40° C. | N | |
| | Peppermint | | | | | | |
| | Oil, 40% PEG | | | | | | |
| | 400 | | | | | | |
| Peppermint | 50% | 40 | | N | Y at 40° C. | Y | >3 days |
| Oil and | Peppermint | | | | | | |
| Tween ® 20 | Oil, 50% | | | | | | |
| | Tween ® 20 | | | | | | |
| | | 60 | | N | Y at 40° C. | Y | >3 days |
| | | 125 | | N | Y at 40° C. | N | |
| Peppermint | 40% | 52 | | N | Y at 40° C. | N | |
| Oil, PEG | Peppermint | | | | | | |
| 400, Glycerol | Oil, 40% PEG | | | | | | |
| | 400, 20% | | | | | | |
| | Glycerol | | | | | | |
| | 45% | 59 | | N | Y at 40° C. | N | |
| | Peppermint | | | | | | |
| | Oil, 45% PEG | | | | | | |
| | 400, 10% | | | | | | |
| | Glycerol | | | | | | |
| Peppermint | 50% | 20 | | Y | | | >3 days |
| Oil and | Peppermint | | | | | | |
| Sesame Oil | Oil, 50% | | | | | | |
| | Sesame Oil | | | | | | |
| | | 40 | | N | Y at 40° C. | Y | >3 days |
| | | 60 | | N | Y at 40° C. | Y | >3 days |
| Peppermint | 33% | 60 | | N | Y at 40° C. | Y | >3 days |
| Oil, Tween ® | Peppermint | | | | | | |
| 20, PEG 400 | Oil, 33% | | | | | | |
| | Tween ® 20, | | | | | | |
| | 33% PEG 400 | | | | | | |
| Soybean Oil, | 50% Soybean | 100 | mg/g | N | Y at 60° C. | waxy | |
| Beeswax | Oil, 50% | | | | | solid | |
| | Beeswax | | | | | | |
| | (w/w) | | | | | | |
| | 75% Soybean | 200 | mg/g | N | Y at 60° C. | waxy | |
| | Oil, 25% | | | | | solid | |
| | Beeswax | | | | | | |
| | (w/w) | | | | | | |
| | 90% Soybean | 200 | mg/g | N | Y at 60° C. | waxy | |
| | Oil, 10% | | | | | solid | |
| | Beeswax | | | | | | |
| | (w/w) | | | | | | |
| | 95% Soybean | 200 | mg/g | N | Y at 60° C. | waxy | |
| | Oil, 5% | | | | | solid | |
| | Beeswax | | | | | | |
| | (w/w) | | | | | | |
| Olive Oil, | 90% Olive | 200 | mg/g | N | Y at 60° C. | waxy | |
| Beeswax | Oil, 10% | | | | | solid | |
| | Beeswax | | | | | | |
| | (w/w) | | | | | | |
| | 95% Olive | 200 | mg/g | N | Y at 60° C. | waxy | |
| | Oil, 5% | | | | | solid | |
| | Beeswax | | | | | | |
| | (w/w) | | | | | | |
| Cinnamaldehyde, | 0.4% (v/v) | 60 | mg/mL | N | Y at 50° C. | Creamy | |
| Olive Oil, | Cinnamaldehyde, | | | | | solid | |
| Beeswax | 5% (w/v) | | | | | | |
| | Beeswax, | | | | | | |
| | 94.5% (v/v) | | | | | | |
| | Olive Oil | | | | | | |
| Eugenol, | 0.5% (v/v) | 100 | mg/mL | N | Y at 50° C. | Creamy | |
| Beeswax, | Eugenol | | | | | solid | |
| Olive Oil | | | | | | | |
| Camillia Oil | 100% | 50 | mg/mL | N | Y at 60° C. | N | >7 days |
| | | 100 | mg/mL | N | Y at 60° C. | Creamy | |
| | | | | | | solid | |

Table 17 shows the results obtained for the solubility of M₄N in water-soluble organic solvents EtOH, PG, PEG 300, PEG 400, PEG 400 monolaurate, glycerol, PVP, and certain combinations thereof.

TABLE 17
Solubility of M₄N in Water-Soluble Organic Solvents

| | Excipient | | | | | |
|---|---|---|---|---|---|---|
| | Concentration | | | | Dissolution | Stability |
| | (v/v unless | Drug | Dissolution | Dissolution | After | Time at |
| | noted | Concentration | After | After | Cool- | Room |
| Excipients | otherwise)) | (in mg/mL) | Rotation | Heating | Down | Temperature |
| Ethanol | 100% | 1 | Y | | | >3 days |
| | | 10 | N | N at | | |
| | | | | 37° C. | | |
| PVP | 15% (w/v) | 1 | N | N at | | |
| | | | | 90° C. | | |
| | | 10 | N | N at | | |
| | | | | 90° C. | | |
| Propylene | 100% (w/v) | 1 | N | Y at | Y | <1 day |
| Glycol | | | | 55° C. | | |
| | | 10 | N | Y at | Y | <1 day |
| | | | | 55° C. | | |
| | | 20 | N | Y at | Y | <1 day |
| | | | | 55° C. | | |
| PEG 400 | 100% | 25 | N | Y at | Y | <7 days |
| | | | | 50° C. | | |
| | | 30 | N | Y at | Y | <3 days |
| | | | | 50° C. | | |
| | | 40 | N | Y at | Y | <1 day |
| | | | | 50° C. | | |
| | | 50 | N | Y at | Y | <1 day |
| | | | | 60° C. | | |
| | | 100 | N | Y at | N | |
| | | | | 60° C. | | |
| | 5% | 10 | N | N at | | |
| | | | | 50° C. | | |
| PEG 400 | 100% | 20 | N | Y at | Y | >3 days |
| mono- | | | | 50° C. | | |
| laurate | | | | | | |
| | | 50 | N | Y at | Y | <1 day |
| | | | | 50° C. | | |
| PEG 300 | 100% | 25 | N | Y at | Y | <7 days |
| | | | | 50° C. | | |
| | | 30 | N | Y at | Y | <3 days |
| | | | | 50° C. | | |
| | | 40 | N | Y at | Y | <1 day |
| | | | | 50° C. | | |
| | | 50 | N | Y at | Y | <1 day |
| | | | | 60° C. | | |
| | | 100 | N | Y at | N | |
| | | | | 60° C. | | |
| | 33% | 10 | N | N at | | |
| | | | | 50° C. | | |
| Glycerol | 100% | 1 | N | Y at | N | |
| | | | | 70° C. | | |
| | | 10 | N | Y at | N | |
| | | | | 70° C. | | |
| | | 20 | N | Y at | N | |
| | | | | 70° C. | | |
| Propylene | 40% Propylene | 10 | N | N | | |
| Glycol and | Glycol | | | | | |
| Ethanol | (w/v), 10% | | | | | |
| | Ethanol (v/v) | | | | | |

### EXAMPLE 6

### Solubility of M₄N in Non-Ionic Surfactants

A 10 mL solution of 20% TPGS (w/v) for use as a solubilizing agent and/or excipient was made as follows: 8 mL of WFI were placed in a glass beaker containing a stir bar. The beaker was placed on a hot magnetic plate, and the stir bar was set to stir at medium speed. The WFI was heated at about 95° C. for 5 minutes. Two grams of TPGS was added to another glass beaker, and heated at about 40° C. for 15 minutes, or until all the TPGS had melted. The melted TPGS was slowly added to the near boiling WFI, using a spatula to direct the TPGS to the center of the beaker so as to prevent any TPGS from sticking to the beaker wall. The 20% TPGS solution was stirred for about 24 hr or until the TPGS was dissolved completely upon visual inspection. The final solution was then stored at room temperature, and was protected from light.

This method of making TPGS may be scaled up or down to obtain the desired volume or concentration of TPGS solution. Other TPGS solutions may be similarly made in combination with other non-ionic surfactants, ionic surfactants, amphipathic surfactants, water-soluble organic solvents, or other solubilizing agents in the process described above. Results are shown in Table 18.

A 10 mL solution of M₄N at a concentration of about 60 mg/mL in Tween® 20 was made as follows. About 10 mL of Tween® 20 were added to a glass beaker containing a stir bar. The beaker was placed on a magnetic plate and the stir bar was set to stir at medium speed. About 600 mg of M₄N were slowly added to the Tween® 20 in the center of the beaker with the aid of a spatula to prevent M₄N from sticking to the beaker wall. The M₄N/Tween®120 mixture was stirred for 2 hr or until all M₄N had dissolved or was uniformly suspended without any clumps being present. The M₄N/Tween® 20 mixture was heated at about 60° C. for about 30 min. (or longer if a larger volume of solution was desired, for example, 1 hr at 60° C. for 500 mL of the M₄N/Tween® 20 mixture), or longer as need to ensure complete dissolution of M₄N. The M₄N/Tween® 20 mixture or solution was observed for presence of any undissolved M₄N by holding the beaker against a white background followed by a dark background, looking for presence of particulates. The final M₄N/Tween® 20 solution was stored at room temperature and was kept protected from light. If crystals were to form during storage, the M₄N/Tween® 20 solution could be heated again at 60° C. for about 1 hr, with stirring, on a hot magnetic plate or until all M₄N was dissolved back in solution.

This process may be scaled up or down to obtain the requisite volume or concentration of M₄N and the heating temperature may be increased or decreased to achieve dissolution of M₄N. Formulations containing M₄N in other non-ionic surfactant, ionic surfactants or amphiphilic molecules may be similarly made, such as, for example, by substituting Tween® 20 in the process described above with Tween® 80, other solubilizing agents, and combinations thereof. Results of the solubility of M₄N in Tween® 20, Tween® 80, and a combination of Tween® 20 and PEG 400 are shown in Table 18.

Table 18 shows that M₄N was soluble in Tween® 20 or Tween® 80 at a concentration of 1 mg/mL at room temperature. The M₄N in Tween® 20 solution (“M₄N/Tween® 20”) was stable at room temperature for more than 7 days, while the M₄N in Tween® 80 solution (“M₄N/Tween® 80”) was stable for more than 3 days of observation. Higher concentrations of M₄N were soluble in Tween® 20 or Tween® 80 at 50° C. Further, M₄N remained in solution after cooling at concentrations of up to 60 mg/mL in Tween® 20 or up to 50 mg/mL in Tween® 80, while becoming insoluble upon cooling at the 80 mg/mL and 100 mg/mL levels in Tween® 20. The 10 mg/mL and 20 mg/mL M₄N/Tween® 20 solutions were observed to be stable at room temperature for greater than 7 days. The 40 mg/mL and 80 mg/mL M₄N/Tween® 20 solutions were observed to be stable at room temperature for less than 3 days. For the M₄N/Tween® 80 solutions, the 10 mg/mL solution was observed to be stable at room temperature for more than 3 days while the 50 mg/mL solution was stable at room temperature for less than 1 day.

Results also show that M₄N was soluble in a combination of 50% Tween® 20 and 50% PEG 400, up to a concentration of 60 mg/mL of M₄N tested when heated at 65° C. M₄N remained in solution in these formulations upon cooling, the solutions being stable at room temperature for more than 3 days.

Combinations of PEG 400 and Tween® 20 were tested at various heating temperatures, at the amount of time heat was added, the method and time of cooling and the concentration of drug added.

A 10 mL solution was made starting with 10 mL of glycerol monooleate (“Glymo”) added to a glass beaker. The beaker was placed on a magnetic plate and the stir bar was set to stir at medium speed. It was heated to 60° C. for 30 minutes to allow all of the drug to dissolve. The mixture was observed for the presence of any undissolved M₄N by holding the beaker against a white background followed by a dark background, looking for presence of particulates. The Glymo mixture was cooled to room temperature with heating and the mixture became a suspension. It was stored at room temperature, protected from light. The suspension remained stable for two weeks and then it began to separate. It could be recombined with shaking.

TABLE 18
Solubility of M₄N in Non-Ionic Surfactants

| | | Drug | | | | |
|---|---|---|---|---|---|---|
| | | Concentration | | | | |
| | | (in | | | | |
| | | mg/ml | | | | |
| | | unless | Dissolution | Dissolution | Dissolution | Stability at |
| | Excipient | otherwise | After | After | After Cool | Room |
| Excipients | Concentration | stated) | Rotation | Heating | Down | Temperature |
| TPGS | 20% (w/v) | 1 | N | Y at 30° C. | Creamy | |
| | | 10 | N | Y at 60° C. | Creamy | |
| | | 125 | N | Y at 60° C. | Creamy | |
| | | 200 | N | Y at 60° C. | Creamy | |
| | 20% (w/v) | 60 | Suspension | | | >3 days |
| | | 100 | Suspension | | | >3 days |
| Tween ® 20 | 100% (v/v) | 1 | Y | | | >7 days |
| | | 10 | N | Y at 50° C. | Y | >7 days |
| | | 20 | N | Y at 50° C. | Y | >7 days |
| | | 40 | N | Y at 50° C. | Y | <3 days |
| | | 60 | N | Y at 50° C. | Y | <3 days |
| | | 80 | N | Y at 50° C. | N | |
| | | 100 | N | Y at 50° C. | N | |
| Tween ® 80 | 100% (v/v) | 1 | Y | | | >3 days |
| | | 10 | N | Y at 50° C. | Y | >3 days |
| | | 50 | N | Y at 50° C. | Y | <1 day |
| Tween ® 20, | 50% Tween ® | 30 | N | Y at 65° C. | Y | >3 days |
| PEG 400 | 20 (v/v), 50% | | | | | |
| | PEG 400 (v/v) | | | | | |
| | | 40 | N | Y at 65° C. | Y | >3 days |
| | | 50 | N | Y at 65° C. | Y | >3 days |
| | | 60 | N | Y at 65° C. | Y | >3 days |
| Tween ® 20, | 47.5% Tween ® | 200 mg/g | N | Y at 65° C. | Waxy solid | >3 days |
| PEG 400, | 20 (v/v), 47.5% | | | | | |
| Beeswax | PEG 400 (v/v), | | | | | |
| | 5% Beeswax | | | | | |
| | (w/v) | | | | | |
| TPGS, PEG | 10% TPGS | 50 | suspension | | | >7 days |
| 400 | (w/v), 50% | | | | | |
| | PEG 400 (v/v) | | | | | |
| PEG 400, | 0.4% (v/v) | 60 mg/mL | N | Y at 60° C. | Y | >4 days |
| Tween ® 20, | Cinnamadehyde, | | | | | |
| Cinnamadehyde | 49.8%(v/v) | | | | | |
| | PEG 400, | | | | | |
| | 49.8% (v/v) | | | | | |
| | Tween ® 20 | | | | | |
| Tween ® 20, | 0.4% (v/v) | 60 mg/mL | N | Y at 50° C. | Y | >2 days |
| PEG 400, | Eugenol, 49.8% | | | | | |
| Eugenol | (v/v) Tween ® | | | | | |
| | 20, 49.8% (v/v) | | | | | |
| | PEG 400 | | | | | |
| Tween ® 20, | 0.4% (v/v) | 60 mg/mL | N | Y at 50° C. | Suspension | |
| PEG 400, | Naringin, | | | | | |
| Naringin | 49.8% (v/v) | | | | | |
| | PEG 400, | | | | | |
| | 49.8% (v/v) | | | | | |
| | Tween ® 20 | | | | | |
| | | 120 mg/mL | N | Y at 50° C. | Suspension | |
| | | 200 mg/mL | N | Y at 50° C. | Suspension | |
| | | 300 mg/mL | N | Y at 50° C. | Suspension | |
| PEG 400, | 25%(w/v) | 60 mg/mL | N | Y at 50° C. | Y | >14 days |
| Tween ® 20, | Lecithin, 37.5% | | | | | |
| Lecithin | (v/v) PEG 400, | | | | | |
| | 37.5% (v/v) | | | | | |
| | Tween ® 20 | | | | | |
| PEG 400, | 3% | 60 mg/mL | N | Y at 80° C. | Y | >24 hours |
| Tween ® 20, | (w/v)Glycerol | | | | | |
| Glycerol | Monostearate, | | | | | |
| Monostearate | 48.5% (v/v) | | | | | |
| | PEG 400, | | | | | |
| | 48.5% (v/v) | | | | | |
| | Tween ® 20 | | | | | |
| PEG 400, | 0.3% (w/v) | 60 mg/mL | N | Y at 50° C. | Creamy | |
| Tween ® 20, | Naringenin, | | | | Solid | |
| Naringenin | 49.8%(v/v) | | | | | |
| | PEG 400 49.8% | | | | | |
| | (v/v) Tween ® | | | | | |
| | 20 | | | | | |
| PEG 400, | 0.4% (v/v) | 60 mg/mL | N | Y at 60° C. | Y | >2 days |
| Tween ® 20, | Cinnamaldehyde, | | | | | |
| Peppermint | 4.6% (v/v) | | | | | |
| Oil, | Peppermint Oil, | | | | | |
| Cinnamaldehyde | 47.5% (v/v) | | | | | |
| | PEG 400, | | | | | |
| | 47.5% (v/v) | | | | | |
| | Tween ® 20 | | | | | |
| PEG 400, | 5% (v/v) | 60 mg/mL | N | Y at 80° C. | Y | >2 days |
| Tween ® 20, | Peppermint Oil, | | | | | |
| Peppermint | 47.5% (v/v) | | | | | |
| Oil | Tween ® 20, | | | | | |
| | 47.5% (v/v) | | | | | |
| | PEG 400 | | | | | |
| PEG 400, | 0.3% (v/v) | 60 mg/mL | N | Y at 55° C. | Suspension | >9 days |
| Tween ® 20, | Piperin, 49.8% | | | | | |
| Piperin | (v/v) PEG 400, | | | | | |
| | 49.8% (v/v) | | | | | |
| | Tween ® 20 | | | | | |
| Tween ® 20, | 47.5% Tween ® | 200 mg/g | N | Y at 65° C. | Waxy solid | >3 days |
| PEG 400, | 20 (v/v), 47.5% | | | | | |
| Beeswax | PEG 400 (v/v), | | | | | |
| | 5% Beeswax | | | | | |
| | (w/v) | | | | | |
| Tween ® 20, | 47.5% (v/v) | 60 mg/mL | N | Y at 65° C. | Y | >4 days |
| PEG 400, | Tween ® 20, | | | | | |
| PEG 3350, | 47.5% (v/v) | | | | | |
| Beeswax | PEG 400, 2.5% | | | | | |
| | (w/v) Beeswax | | | | | |
| | 2.5% (w/v) | | | | | |
| | PEG 3350 | | | | | |
| Tween ® 20, | 50.3% (v/v) | 60 mg/mL | N | Y at 65° C. | Y | >4 days |
| PEG 400, | Tween ® 20, | | | | | |
| PEG 3350, | 44.6% (v/v) | | | | | |
| Beeswax, | PEG 400, 2.4% | | | | | |
| Naringenin | (w/v) Beeswax, | | | | | |
| | 2.4% (w/v) | | | | | |
| | PEG 3350, | | | | | |
| | 0.3% (w/v) | | | | | |
| | Naringenin | | | | | |
| Tween ® 20, | 50% (v/v) | 60 mg/mL | N | Y at 60° C. | Creamy | |
| PEG 400, | Tween ® 20 | | | | Solid | |
| PEG 3350, | 40% (v/v) PEG | | | | | |
| Beeswax, | 400, 5% (w/v) | | | | | |
| Lecithin | Lechitin, 2.5% | | | | | |
| | (w/v) PEG | | | | | |
| | 3350, 2.5% | | | | | |
| | (w/v) Beeswax | | | | | |
| TPGS, PEG | 10% TPGS | 50 | suspension | | | >7 days |
| 400 | (w/v), 50% | | | | | |
| | PEG 400 (v/v) | | | | | |
| Glycerol | 100% (w/v) | 0.10 | N | Y at 60° C. | Suspension | Remains in |
| Monoleate | | | | | | suspension |
| | | | | | | for up to 10 |
| | | | | | | days. Can be |
| | | | | | | mixed back |
| | | | | | | together |
| | | 0.25 | N | Y at 60° C. | Suspension | Remains in |
| | | | | | | suspension |
| | | | | | | for up to 10 |
| | | | | | | days. Can be |
| | | | | | | mixed back |
| | | | | | | together |
| | | 1.00 | N | Y at 60° C. | Suspension | Remains in |
| | | | | | | suspension |
| | | | | | | for up to 10 |
| | | | | | | days. Can be |
| | | | | | | mixed back |
| | | | | | | together |
| | | 5.0 | N | Y at 60° C. | Suspension | Remains in |
| | | | | | | suspension |
| | | | | | | for up to 10 |
| | | | | | | days. Can be |
| | | | | | | mixed back |
| | | | | | | together |
| | | 10.0 | N | Y at 60° C. | Suspension | Remains in |
| | | | | | | suspension |
| | | | | | | for up to 10 |
| | | | | | | days. Can be |
| | | | | | | mixed back |
| | | | | | | together |
| | | 12.0 | N | Y at 60° C. | Suspension | Remains in |
| | | | | | | suspension |
| | | | | | | for up to 10 |
| | | | | | | days. Can be |
| | | | | | | mixed back |
| | | | | | | together |
| | | 15.0 | N | Y at 60° C. | Suspension | Remains in |
| | | | | | | suspension |
| | | | | | | for up to 10 |
| | | | | | | days. Can be |
| | | | | | | mixed back |
| | | | | | | together |
| | | 17.5 | N | Y at 60° C. | Suspension | Remains in |
| | | | | | | suspension |
| | | | | | | for up to 10 |
| | | | | | | days. Can be |
| | | | | | | mixed back |
| | | | | | | together |
| | | 20.0 | N | Y at 60° C. | Suspension | Remains in |
| | | | | | | suspension |
| | | | | | | for up to 10 |
| | | | | | | days. Can be |
| | | | | | | mixed back |
| | | | | | | together |
| | | 25.0 | N | Y at 60° C. | Suspension | Remains in |
| | | | | | | suspension |
| | | | | | | for up to 10 |
| | | | | | | days. Can be |
| | | | | | | mixed back |
| | | | | | | together |
| | | 30.0 | N | Y at 60° C. | Suspension | Remains in |
| | | | | | | suspension |
| | | | | | | for up to 10 |
| | | | | | | days. Can be |
| | | | | | | mixed back |
| | | | | | | together |
| | | 40.0 | N | Y at 60° C. | Suspension | Remains in |
| | | | | | | suspension |
| | | | | | | for up to 10 |
| | | | | | | days. Can be |
| | | | | | | mixed back |
| | | | | | | together |
| | | 50.0 | N | Y at 60° C. | Suspension | Remains in |
| | | | | | | suspension |
| | | | | | | for up to 10 |
| | | | | | | days. Can be |
| | | | | | | mixed back |
| | | | | | | together |
| | | 60.0 | N | Y at 60° C. | Suspension | Remains in |
| | | | | | | suspension |
| | | | | | | for up to 10 |
| | | | | | | days. Can be |
| | | | | | | mixed back |
| | | | | | | together |
| | | 65.0 | N | Y at 60° C. | Suspension | Remains in |
| | | | | | | suspension |
| | | | | | | for up to 10 |
| | | | | | | days. Can be |
| | | | | | | mixed back |
| | | | | | | together |
| | | 70.0 | N | Y at 60° C. | Suspension | Remains in |
| | | | | | | suspension |
| | | | | | | for up to 10 |
| | | | | | | days. Can be |
| | | | | | | mixed back |
| | | | | | | together |
| | | 80.0 | N | Y at 60° C. | Suspension | Remains in |
| | | | | | | suspension |
| | | | | | | for up to 10 |
| | | | | | | days. Can be |
| | | | | | | mixed back |
| | | | | | | together |
| | | 100.0 | N | Y at 60° C. | Suspension | Remains in |
| | | | | | | suspension |
| | | | | | | for up to 10 |
| | | | | | | days. Can be |
| | | | | | | mixed back |
| | | | | | | together |
| | | 200.0 | N | Y at 60° C. | Suspension | Remains in |
| | | | | | | suspension |
| | | | | | | for up to 10 |
| | | | | | | days. Can be |
| | | | | | | mixed back |
| | | | | | | together |
| | | 300.0 | N | Y at 60° C. | Suspension | Remains in |
| | | | | | | suspension |
| | | | | | | for up to 10 |
| | | | | | | days. Can be |
| | | | | | | mixed back |
| | | | | | | together |
| PEG 400, | 25% (v/v) PEG | 60 mg/mL | N | Y at 50° C. | N- | |
| Tween ® 20, | 400, 25% (v/v) | | | | separated | |
| Glycerol | Tween ® 20, | | | | into 2 | |
| Monooleate | 50% (v/v) | | | | separate | |
| | Glycerol | | | | layers | |
| | Monooleate | | | | | |
| | 30% (v/v) PEG | 60 mg/mL | N | Y at 50° C. | N- | |
| | 400, 30% (v/v) | | | | separated | |
| | Tween ® 20, | | | | into 2 | |
| | 40% (v/v) | | | | separate | |
| | Glycerol | | | | layers | |
| | Monooleate | | | | | |
| Glycerol | 15% (w/v) | 60 mg/mL | N | N | | |
| Monostearate | solution in | | | | | |
| | water | | | | | |
| Gelucire ® | 100% | 60 mg/mL | N | Y at 60° C. | Creamy | |
| 44/14 | | | | | solid | |
| | | 200 mg/mL | N | Y at 60° C. | Creamy | |
| | | | | | solid | |
| | | 300 mg/mL | N | Y at 60° C. | Creamy | |
| | | | | | solid | |
| Labrasol ® | 100% | 50 mg/mL | N | Y at 60° C. | Y | >4 days |
| | | 60 mg/mL | N | Y at 60° C. | Y | >4 days |
| | | 70 mg/mL | N | Y at 60° C. | Y | >4 days |
| | | 80 mg/mL | N | Y at 60° C. | Y | >4 days |
| | | 90 mg/mL | N | Y at 60° C. | suspension | |
| | | 100 mg/mL | N | Y at 60° C. | suspension | |
| | | 200 mg/mL | N | Y at 60° C. | suspension | |
| Labrafil ® | 100% | 60 mg/mL | N | Y at 60° C. | Y | >4 days |
| | | 200 mg/mL | N | Y at 60° C. | Creamy | |
| | | | | | solid | |

### EXAMPLE 7

The melting point of the 47.5% (v/v) Tween® 20, 47.5% (v/v) PEG 400, 2.5% (w/v) PEG 3350, 2.5% (w/v) Beeswax of Example 6 (see Table 18) was determined for several different concentrations of M₄N, as follows:

| Concentration of M₄N | Melting Temperature |
|---|---|
| 0% | 47.5° C. |
| 5% | 47.5° C. |
| 10% | 48° C. |
| 20% | 55° C. |
| 30% | 55° C. |
| 40% | 62° C. |
| 50% | 62° C. |

### EXAMPLE 8

### Lyophilized Formulations Containing M₄N in HP-β-CD

A 120 mg lyophilized powder of M₄N at a concentration of about 185 mg/g (w/w) in HP-β-CD was made as follows. Equal molar amounts of HP-β-CD and M₄N were used to increase the complexation rate between HP-β-CD and M₄N. About 98 mg of HP-β-CD and about 22.2 mg M₄N were mixed together in a 1.5 mL-sized polypropylene tube. About 0.2 mL WFI was added to the mixture in the polypropylene tube containing the HP-β-CD/M₄N powder mixture and vortexed for 1 minute to produce a HP-β-CD/M₄N suspension in water. The HP-β-CD/M₄N suspension was frozen at −20° C. for 24 hours. The HP-β-CD/M₄N suspension was then centrifuged at 1,400 rpm under vacuum at 60° C. for about 2 hours to remove all the water from the suspension. The dry powder of HP-β-CD/M₄N complex weighed about 120 mg. This HP-β-CD/M₄N powder complex may be then dissolved or resuspended in water or other solubilizing agents. The final M₄N/HP-β-CD powder complex was stored at room temperature and was kept protected from light. This process may be scaled up or down to obtain the requisite volume or concentration of M₄N. Formulations containing M₄N with other cyclodextrins may be similarly made, such as, for example, by substituting HP-β-CD in the process described above with other cyclodextrins. Results shown in Table 1 demonstrate that a powder complex of HP-β-CD/M₄N was obtained after lyophilization of the HP-β-CD/M₄N suspension consisting of about 81.5% HP-β-CD and 18.5% M₄N.

A 400 mg lyophilized powder of M₄N at a concentration of about 150 mg/g (w/w) in HP-β-CD/HPMC was made as follows. About 1 mL of a 50% HP-β-CD/0.5% HPMC solution, made as described above, was added to a 1.5 mL-sized polypropylene tube. About 60 mg M₄N were added to the polypropylene tube containing the HP-β-CD/HPMC suspension and vortexed for 1 minute to produce a HP-β-CD/HPMC/M₄N suspension. The HP-β-CD/HPMC/M₄N suspension was frozen at −20° C. for 24 hours. The HP-β-CD/HPMC/M₄N suspension was then centrifuged at 1,400 rpm under vacuum at 60° C. for about 5 hours to remove all the water from the suspension. The dry powder of HP-β-CD/HPMC/M₄N complex weighed about 400 mg. This HP-β-CD/HPMC/M₄N powder complex may be then dissolved or resuspended in water or other solubilizing agents. The final M₄N/HP-β-CD/HPMC powder complex was stored at room temperature and was kept protected from light. This process may be scaled up or down to obtain the requisite volume or concentration of M₄N. Formulations containing M₄N with other cyclodextrins/cellulose solutions may be similarly made, such as, for example, by substituting HP-β-CD in the process described above with other cyclodextrins, or by substituting HPMC with other modified celluloses. Results shown in Table 15 demonstrate that a powder complex of HP-β-CD/HPMC/M₄N was obtained after lyophilization of the HP-β-CD/HPMC/M₄N suspension consisting of about 84% HP-β-CD, 1% HPMC and 15% M₄N.

### EXAMPLE 9

### Absorption of M₄N in Sprague Dawley Rats Upon Oral Administration of a Liquid Formulation

Ten groups of male rats (n=5 per group) (age=8-10 weeks) were exposed by oral gavage to a single 500 mg/kg dose of M₄N in excipient to determine the optimal oral liquid excipient. The animals were fasted overnight prior to dosing. The excipients/formulation tested were: (a) HP-β-CD+HPMC; (b) HP-β-CD+CMC; (c) TPGS; (d) TPGS+PEG 400; (e) Tween® 20; (f) PEG 400+Tween® 20; (g) PEG 400+Tween® 20+peppermint oil; (h) peppermint oil+PEG 400; (i) peppermint oil+Tween® 20; (j) peppermint oil+sesame oil, as shown in Table 19, using the formulations made as previously described. Blood was collected from each animal via the jugular vein at the following time points: predose, 0.5, 1, 2 and 3 hr post dosing. Concentrations of M₄N in serum were determined by LC/MS/MS, LC meaning liquid chromatography, MS meaning mass spectrometry. This method analyzes M₄N that has been separated by LC, then detected and quantified by two consecutive rounds of MS.

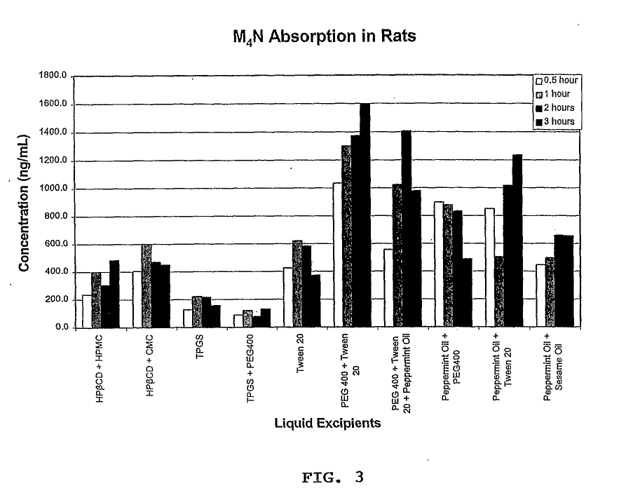

As shown in FIG. 3 and Table 20, M₄N absorption varied between excipient formulations. Absorption was highest for PEG 400+Tween® 20, which was higher than PEG 400+Tween® 20+peppermint oil, which was higher than peppermint oil+Tween® 20, which was higher than peppermint oil+PEG 400, which was higher than peppermint oil+sesame oil, which was higher than or equal to Tween® 20, which was higher than or equal to HP-β-CD+CMC, which was higher than HP-β-CD+HPMC, which was higher than TPGS, which was higher than TPGS+PEG 400.

In Table 20, four formulations were indicated as stable suspensions: HP-β-CD+HPMC, HP-β-CD+CMC, TPGS, and TPGS+PEG 400. The criterion used for designating those suspensions as “stable” was that the suspensions did not show precipitation of their contents (the ingredients did not settle down at the bottom of the beaker from the suspension). The other formulations in the rat study were clear liquid solutions, not suspensions, and did not have any floating ingredients.

TABLE 19
Oral Formulations for Absorption Studies

| | | |
|---|---|---|
| | | M₄N |
| GROUP | ITEM DESCRIPTION | CONCENTRATION |

Rat Excipient Study

| | | | |
|---|---|---|---|
| | 500 mg/mL HP-β-CD, | | |
| 1 | 5 mg/mL HPMC | 60 | mg/mL* |
| | 500 mg/mL HP-β-CD, | | |
| 2 | 5 mg/mL CMC | 60 | mg/mL* |
| 3 | 200 mg/mL TPGS | 60 | mg/mL* |
| | 100 mg/mL TPGS | | |
| 4 | 50% PEG400 (v/v) | 60 | mg/mL* |
| 5 | 100% Tween ® 20 (v/v) | 60 | mg/mL |
| 6 | 50% PEG400 (v/v), 50% Tween ® | 60 | mg/mL |
| | 20 (v/v) | | |
| 7 | 33% PEG400 (v/v), 33% Tween ® | 60 | mg/mL |
| | 20 (v/v), 33% Peppermint Oil (v/v) | | |
| 8 | 50% Peppermint Oil (v/v), 50% | 60 | mg/mL |
| | PEG400 (v/v) | | |
| 9 | 50% Peppermint Oil (v/v), | 60 | mg/mL |
| | 50% Tween ® 20 (v/v) | | |
| 10 | 50% Peppermint Oil (v/v), | 60 | mg/mL |
| | 50% Sesame Oil (v/v) | | |

Dog Excipient Study

| 1 | M₄N | powder | |
|---|---|---|---|
| 2 | Lyophilized HP-β-CD | 185 | mg/g (w/w) |
| 3 | 20% TPGS (w/v) | 133 | mg/g (w/w) |
| 4 | 95% Soybean Oil (v/v), 5% | 200 | mg/g (w/w) |
| | Beeswax (w/v) | | |
| 5 | 95% Olive Oil (v/v), 5% Beeswax (w/v) | 200 | mg/g (w/w) |

*Stable suspensions

TABLE 20
Absorption of M₄N in Rats^{a} upon Oral Delivery

| | Concentration (ng/mL) | | | |
|---|---|---|---|---|
| Excipient/Formulation | 0.5 hour | 1 hour | 2 hours | 3 hours |
| HP-β-CD + HPMC | 238 ± 39^{b} | 398 ± 89 | 305 ± 48 | 482 ± 128 |
| HP-β-CD + CMC | 403 ± 116 | 601 ± 125 | 470 ± 96 | 448 ± 94 |
| TPGS | 127 ± 45 | 222 ± 57 | 216 ± 57 | 156 ± 36 |
| TPGS + PEG400 | 87 ± 14 | 120 ± 17 | 77 ± 13 | 130 ± 45 |
| Tween ® 20 | 424 ± 138 | 620 ± 211 | 581 ± 149 | 371 ± 61 |
| PEG400 + Tween ® 20 | 1033 ± 287 | 1300 ± 425 | 1371 ± 429 | 1598 ± 379 |
| PEG400 + Tween ® 20 + Peppermint Oil | 557 ± 169 | 1023 ± 335 | 1404 ± 719 | 977 ± 546 |
| Peppermint Oil + PEG400 | 896 ± 228 | 876 ± 156 | 832 ± 239 | 487 ± 133 |
| Peppermint Oil + Tween ® 20 | 851 ± 346 | 502 ± 96 | 1017 ± 683 | 1233 ± 722 |
| Peppermint Oil + Sesame Oil | 445 ± 193 | 496 ± 137 | 657 ± 143 | 655 ± 264 |

^{a}n = 5 males/group
^{b}Values are expressed as means ± standard errors

### EXAMPLE 10

### Absorption of M₄N in Beagle Dogs Upon Oral Administration

Five groups of dogs (n=2 per group, one male and one female) (age=approximately 6-9 months old) were exposed to a single 100 mg/kg dose of M₄N in excipient to determine the optimal oral solid excipient. The composition of M₄N in excipient was encapsulated in size 12 hard gelatin capsules prior to oral administration. The animals were fasted overnight prior to dosing. The excipients/formulations tested are set forth in Table 19 and were: (a) no excipient, where M₄N was encapsulated; (b) HP-β-CD; (c) TPGS; (d) soybean oil+beeswax; and (e) olive oil+beeswax. Blood was collected from each animal via the jugular vein at the following time points: predose, 0.5 hr, 1 hr, 1.5 hr, 2 hr, 4 hr, 6 hr and 8 hr post dosing. Concentrations of M₄N in serum were determined by Liquid Chromatography combined with tandem Mass Spectroscopy (LC/MS/MS).

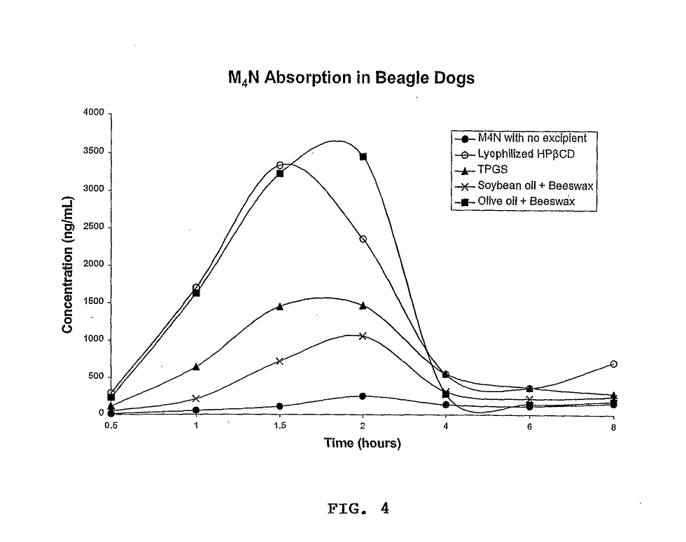

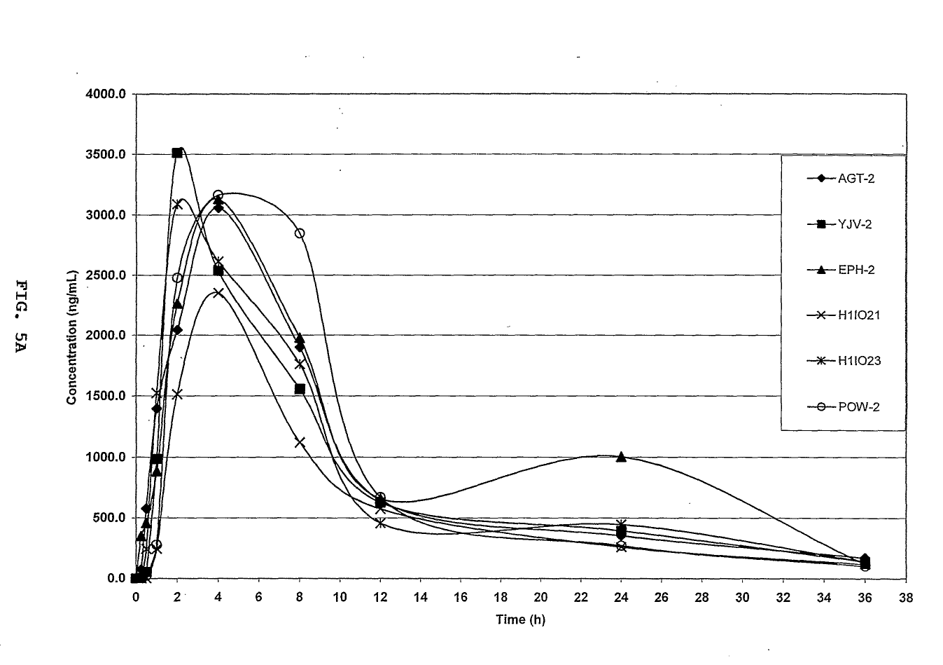

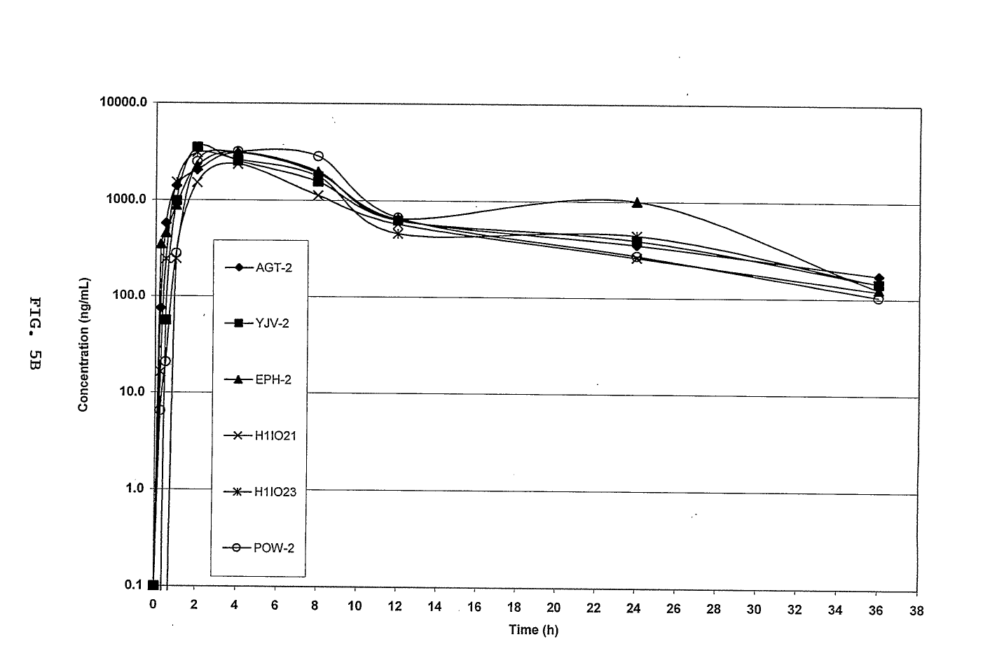

As shown in FIG. 4, absorption of M₄N varied depending on the excipient/formulation. Absorption was highest for the formulation containing olive oil+beeswax, which was higher than that containing HP-β-CD, which was higher than that containing TPGS, which was higher than that containing soybean oil+beeswax, which was higher than M₄N without an excipient.

Although the foregoing examples are illustrated with M₄N, other NDGA derivatives or other catecholic butanes may be substituted therein with appropriate adjustments for weights and volumes to achieve the appropriate concentrations.

### EXAMPLE 11

### Collection of Samples for Determination of the Pharmacokinetics of Micronized and Non-Micronized M₄N after Oral Administration to Dogs

The objective of this study was to evaluate the pharmacokinetic profile of M₄N when administered in a micronized or non-micronized form via oral gavage dosing in beagle dogs. The test articles were prepared in glycerol monooleate at 60 mg/mL. The study design is summarized in Table 21.

TABLE 21
Study Design for Non-Micronized and Micronized M₄N Testing in Dogs

| | | | | Target | | |
|---|---|---|---|---|---|---|
| | Number | | | Dose | Target Dose | Target Dose |
| Group/ | of | Test | Dose | Level | Concentration | Volume |
| Phase | Animals | Article | Route | (mg/kg) | (mg/mL) | (mL/kg) |
| 1/1 | 3 M, 3 F | Non-micronized M₄N | Oral | 100 | 60 | 1.67 |
| 1/2 | 3 M, 3 F | Micronized M₄N | Oral | 100 | 60 | 1.67 |

M Male
F Female
Note:
There was a washout period of approximately 7 days between phases.

Blood (approximately 2 mL) was collected from a jugular vein into Monoject 2 mL red top tubes containing no anticoagulant predose and at 0.25 hr, 0.5 hr, 1 hr, 2 hr, 4 hr, 8 hr, 12 hr, 24 hr, and 36 hr postdose.

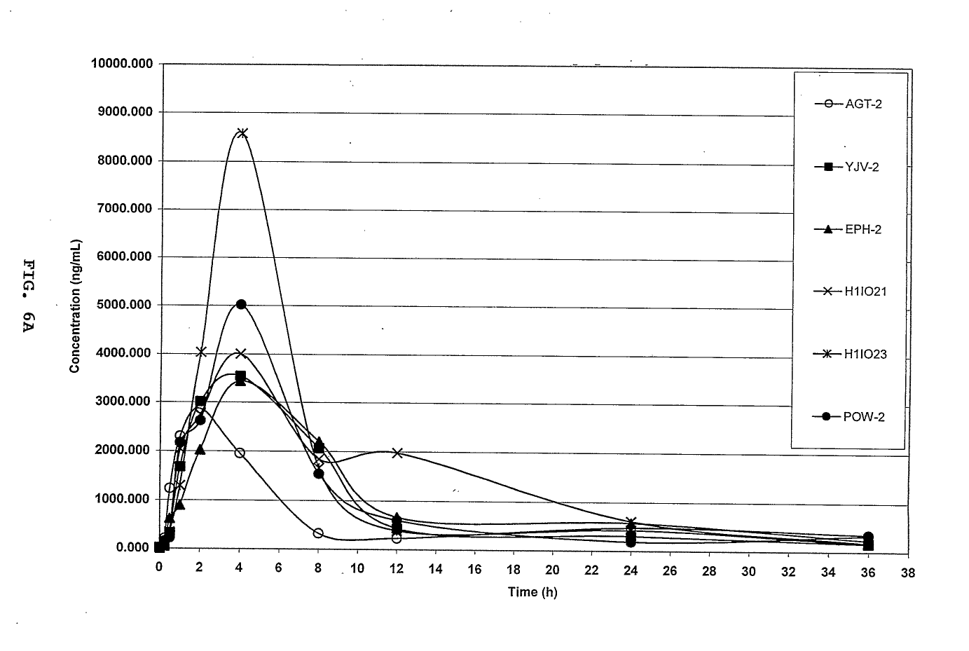

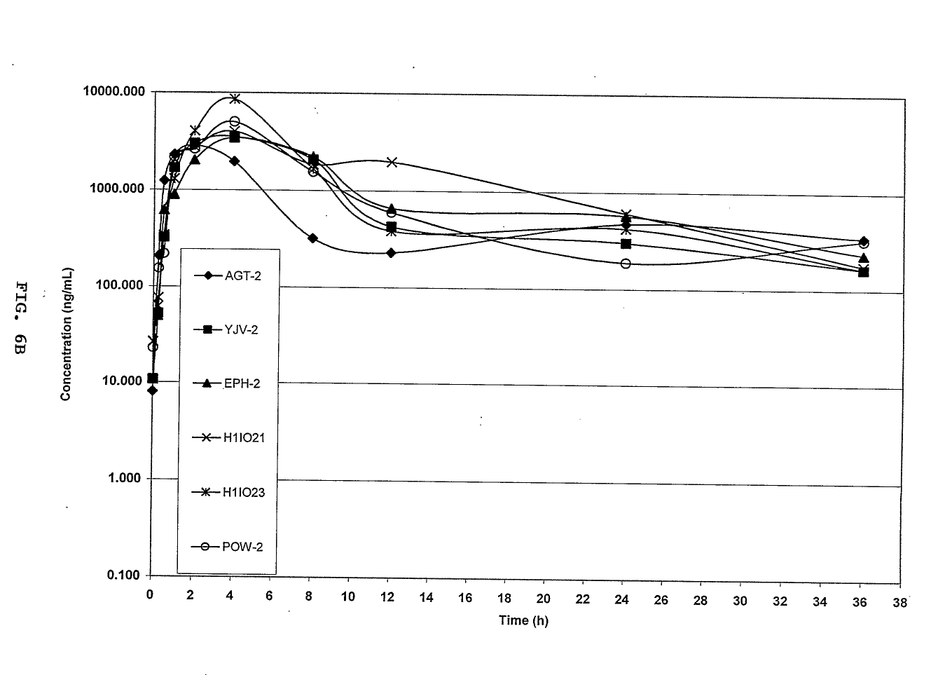

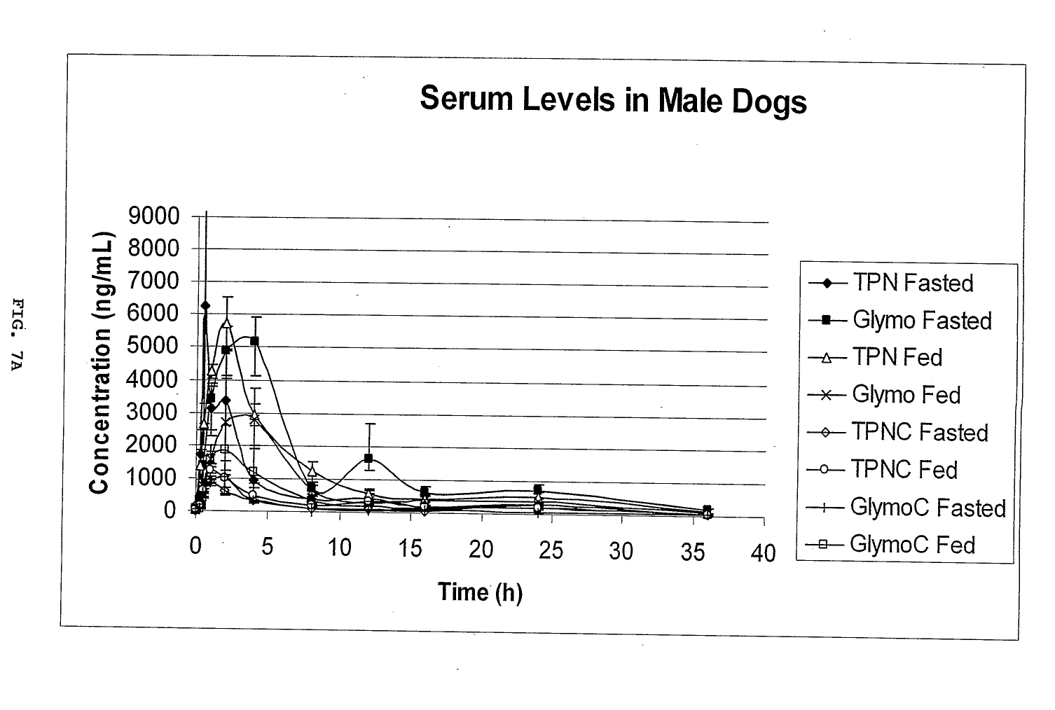

Absorption of M₄N occurred after administration of both formulations. Mean pharmacokinetic parameters are presented in Table 22, where Cₘₐₓ is the maximum concentration of M₄N absorbed and AUC represents the areas under curve, relating to the total M₄N absorbed. Absorption was higher after administration of micronized M₄N compared to non-micronized M₄N. However concentration curves tended to be more consistent among animals after administration of non-micronized M₄N (FIGS. 5A and 5B, non-logarithmic and logarithmic scales, respectively, showing absorption of non-micronized M₄N; and FIGS. 6A and 6B, non-logarithmic and logarithmic scales, respectively, showing absorption of micronized M₄N).

TABLE 22
Mean Pharmacokinetic Parameters following administration of M₄N

| | Non-Micronized | |
|---|---|---|
| | EM-1421 | Micronized EM-1421 |
| Cₘₐₓ (ng/mL) | 3051 | 4574 |
| Standard Deviation | 380 | 2088 |
| % CV | 12.5 | 45.8 |
| Tₘₐₓ (h) | 3.3 | 3.7 |
| Standard Deviation | 1.0 | 0.8 |
| % CV | 31.0 | 22.3 |
| AUC_{0-36 h} (ng * h/mL) | 31470 | 37773 |
| Standard Deviation | 5607 | 9920 |
| % CV | 17.8 | 26.3 |
| AUC_{infinity} | 32572 | 40113 |
| Standard Deviation | 5683 | 8809 |
| % CV | 17.4 | 22.0 |

### EXAMPLE 11

### Oral Fed/Fasted IV/Oral Pharmacokinetic Comparison of Two Formulations of M₄N in Beagle Dogs

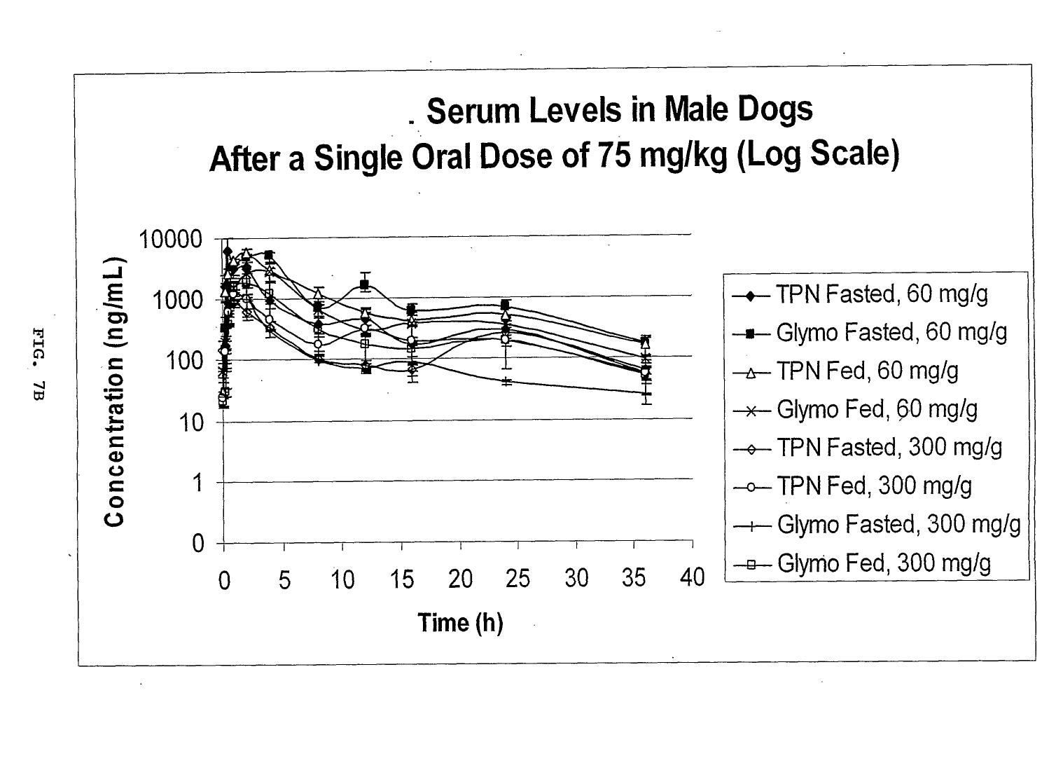

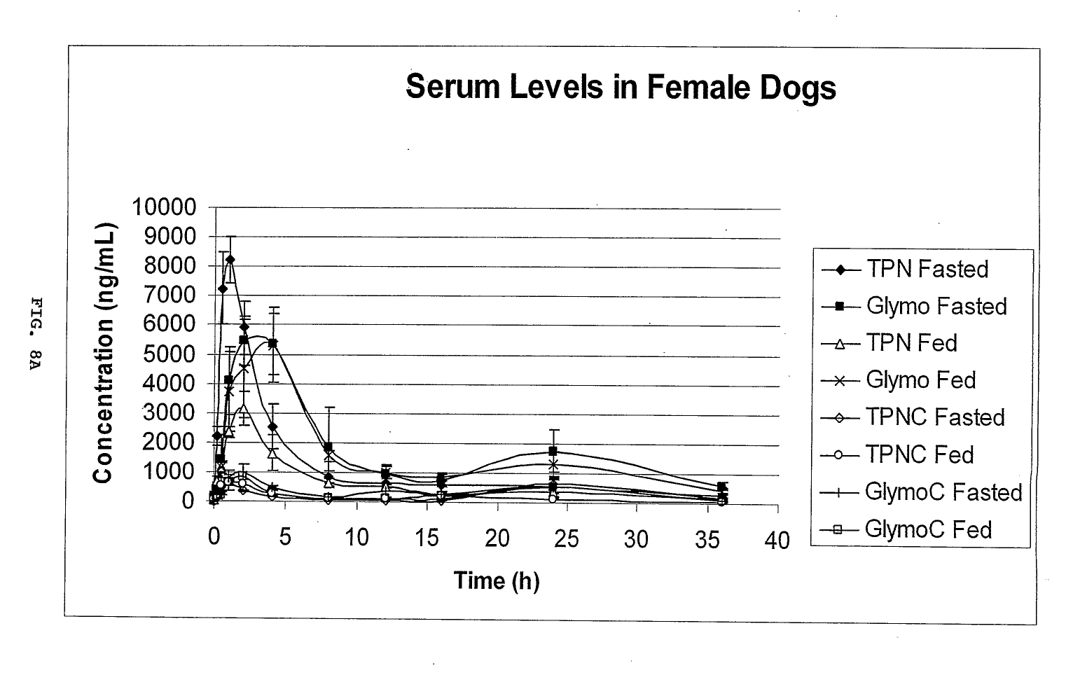

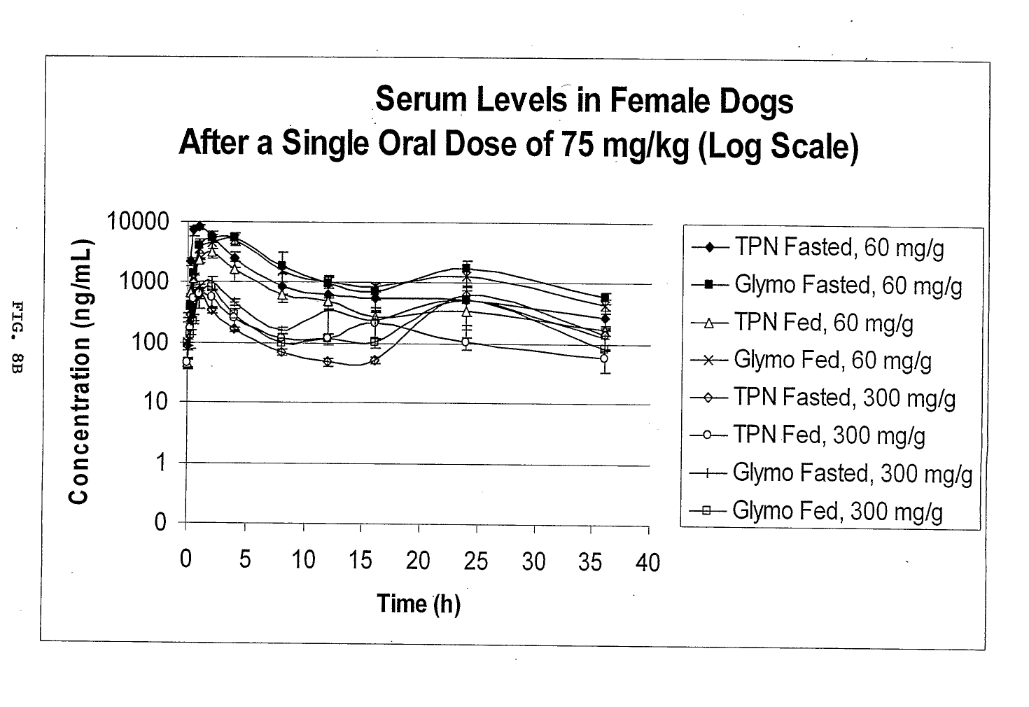

The purpose of this study was to evaluate serum levels achieved after a single 75 mg/kg dose of preformulated M₄N administered orally (by gavage or capsule) under fed or fasted conditions or administered IV. Three dogs/sex were administered EM-1421 in glycerol monooleate (“Glymo”), M₄N in Tween® 20/PEG 400/Naringin (“TPN”), or M₄N in 20% hydroxypropyl-β-cyclodextrin (HPβCD) in 50% PEG 300 (“CPE”). Glymo and TPN were also administered as non-concentrated (60 mg/mL) or concentrated (300 mg/g, w/w), as indicated in the legends to FIGS. 7B and 8B.

All animals survived throughout the study. Clinical observations included diarrhea, emesis, and ataxia (ataxia occurred in 1 female after IV dosing and lasted for approximately 1 hour).

Serum levels of M₄N varied greatly with formulation and condition. Table 23 summarizes these findings. Serum levels were lowest after administration with concentrated Glymo or TPN. Cₘₐₓ was highest after administration of TPN (non-concentrated, fasted condition) (Table 23 and FIGS. 7A and 8A, non-logarithmic scales showing serum levels of M₄N orally administered to male and female dogs, respectively). AUCs were highest after administration of Glymo (non-concentrated, fasted condition) (Table 23 and FIGS. 7B and 8B, logarithmic scales showing serum levels of M₄N orally administered to male and female dogs, respectively). AUCs achieved after oral administration of Glymo (non-concentrated, fasted condition) were 35% (males) and 47% (females) of the AUCs achieved after IV dosing, where IV dosing is assumed to be 100% (Table 23).

TABLE 23
Pharmacokinetics of Formulated M₄N in Dogs
after Single 75 mg/kg Dose

| | | | | Cₘₐₓ | AUC |
|---|---|---|---|---|---|
| Formulation | Administration | Condition | Sex | (ng/mL) | (ng * h/mL) |
| CPE | IV | Fed | M | 37671 | 134103 |
| CPE | IV | Fed | F | 40580 | 137547 |
| TPN | Oral (Gavage) | Fasted | M | 6256 | 20792 |
| TPN | Oral (Gavage) | Fasted | F | 8221 | 41975 |
| TPN | Oral (Gavage) | Fed | M | 5712 | 37722 |
| TPN | Oral (Gavage) | Fed | F | 3144 | 22758 |
| Glymo | Oral (Gavage) | Fasted | M | 5157 | 46938 |
| Glymo | Oral (Gavage) | Fasted | F | 5439 | 64362 |
| Glymo | Oral (Gavage) | Fed | M | 2807 | 24367 |
| Glymo | Oral (Gavage) | Fed | F | 5300 | 57168 |
| TPN-Conc | Oral (Capsule) | Fasted | M | 890 | 7070 |
| TPN-Conc | Oral (Capsule) | Fasted | F | 695 | 9635 |
| TPN-Conc | Oral (Capsule) | Fed | M | 1232 | 9559 |
| TPN-Conc | Oral (Capsule) | Fed | F | 638 | 5924 |
| Glymo-Conc | Oral (Capsule) | Fasted | M | 1020 | 5092 |
| Glymo-Conc | Oral (Capsule) | Fasted | F | 1001 | 13185 |
| Glymo-Conc | Oral (Capsule) | Fed | M | 1851 | 12890 |
| Glymo-Conc | Oral (Capsule) | Fed | F | 817 | 10074 |

### EXAMPLE 12

### Oral (Gavage) Repeated Dose Pharmacokinetic Study of M₄N in Rats

The purpose of this study was to provide information on pharmacokinetics of a 500 mg/kg dose of M₄N in ten different excipient formulations. Fifty Crl:(CD)SD male and female rats were assigned to ten dosage groups, five rats per sex per group. Samples were prepared having carriers as follows that were administered to the following groups of subjects:

| | |
|---|---|
| Group I | PEG400/Tween ® 20 |
| Group II | PEG400/Tween20 ®/Naringin |
| Group III | PEG400/Tween20 ®/Naringenin |
| Group IV | PEG400/Tween20 ®/Glyceryl Monostearate |
| Group V | Glycerol Monooleate |
| Group VI | PEG400/Tween20 ®/Piperine |
| Group VII | PEG400/Tween20 ®/PEG3350/Beeswax/Naringin |
| Group VIII | PEG400/Tween20 ®/Cinnamaldehyde |
| Group IX | PBG400/Tween20 ®/Peppermint Oil |
| Group X | PEG400/Tween20 ®/PEG3350/Beeswax |

Each formulation of M₄N was administered orally via gavage on three occasions. The concentration of the M₄N was 60 mg/mL administered at a dosage volume 8.33 mL/kg, based on the most recent body weight. The rats were given the first dose (day 1 of study) after being fasted overnight followed by a 72 hour washout (recovery) period. On day 5 of study (DS 5), the second dose of test article was administered to non-fasted rats followed by a one week washout (recovery) period. On DS 6, the rats were placed on a high fat diet (approximately 10% fat content versus approximately 5% in a standard diet). On DS 12, the non-fasted rats were administered the third dose. The rats were observed for viability at least twice daily and for clinical observations and general appearance weekly during the acclimation period. The rats were also examined for clinical observations, deaths before dosage and at approximately hourly intervals for the first four hours post dosage and at the end of the normal working day on the first day of dosage administration, and at approximately 2 hours on subsequent days of dosage administration. These observations were also recorded once daily on non dosing days and during the post dosage period. Body weights were recorded at least weekly during the acclimation period, daily during the dosage and at sacrifice terminal weight. Feed consumption values were recorded weekly during the dosage period and at sacrifice (feed left value). On days 1, 5 and 12 of the study, blood samples (approximately 0.5 mL each) were collected via the lateral tail vein from each rat assigned to the study. The time of each blood collection was recorded in the raw data. Blood samples were collected from each rat on days 1, 5, and 12 of study at the following time points: prior to dosage, 15 minutes post dosage, and 1 and 4 hours post dosage. The samples were transferred into serum separator tubes and spun in a centrifuge. The resulting serum was transferred into polypropylene tubes labeled with the protocol number, Sponsor study number, rat number, group number, dosage level, day of study, collection interval, date of collection, species, generation and storage conditions. All samples were immediately frozen on dry ice and maintained frozen (−68° C. to −78° C.) until shipment for analysis.

All rats survived to their scheduled sacrifice. Urine stained abdominal fur occurred in four of the five female rats administered PEG 400/Tween® 20/peppermint oil (Group IX). This sign occurred on DSs 2 to 3 and did not persist. Soft or liquid feces was the only clinical observation that occurred in at least a few rats in each group with the exception of PEG 400/Tween® 20/peppermint oil (Group IX) rats. All groups had average weight gains during the study. Weight gains for the male and female rats for DSs 6 to 13 when the high fat diets were fed were always less than for DSs 1 to 6 when the standard diet was fed. Weight gains were generally comparable among the groups with the exception of the female rats on DSs 6 to 13 in the PEG 400/Tween® 20 and PEG 400/Tween® 20/PEG 3350/beeswax dosage groups (Groups I and X, respectively) that had reduced body weight gains. Absolute and relative feed consumption values were comparable among the groups throughout the study. No gross lesions related to the test article formulations were observed at necropsy. Absorption of M₄N occurred from all vehicles. Blood levels of rats on a high fat diet were always higher than those achieved on a standard diet or after fasting. The highest absorption occurred in the PEG 400/Tween® 20/naringin group after administration of a high fat diet. The highest absorption occurred in the PEG 400/Tween® 20/naringenin group after administration of a standard diet. The highest absorption occurred in the PEG 400/Tween® 20/peppermint oil group after fasting conditions. The highest exposure levels were generally achieved within one hour post dose, except for the glycerol monooleate formulation, which appeared to be continuing to rise at four hours post dose.

In conclusion, all test article formulations were tolerated with no mortality and all groups gained weight. Soft and liquid feces was the most common clinical observation but no effect on feed consumption occurred for any vehicle. Blood levels of M₄N were always higher when rats were fed the high fat diet. Peak exposure occurred within one hour post dosage with the exception of the glycerol monooleate formulation.

### EXAMPLE 13

### A Human Phase 0, Three-Way Crossover Microdose Pharmacokinetic Study of ¹⁴C-Labelled M₄N in Eight Healthy Male Subjects

This study was designed to assess the absorption of M₄N when administered to humans as a sub-therapeutic dose either as a single oral dose under fed and fasted conditions or a single intravenous dose (Regimens A-C, respectively, in Table 24). The study was a three-way crossover study design in a target population of healthy male subjects and consisted of three study periods of approximately 35 hours duration, each separated by a minimum period of at least 7 days between dosing. During the course of each study period, pharmacokinetic blood samples were taken at specified time points after dosing and urine was collected over pre-defined time intervals. The subjects were able to leave the clinical unit after the completion of study specific procedures at 24 hours post-dose.

In this study, M₄N was administered to humans in a 100 μg quantity. M₄N was lightly-labelled with ¹⁴C (3.3 kBq per 100 μg) and administered to healthy volunteers. Each oral administration of M₄N consisted of 0.1 mg of ¹⁴C labeled M₄N and 376.8 mg of glycerol monooleate in a size 0 gelatin capsule. The single intravenous infusion of M₄N consisted of 0.1 mg/mL ¹⁴C labelled M₄N, 30% (w/v) HP-β-CD, and 25% (v/v) PEG diluted with water to 1 mL for bolus injection. Following collection of blood and urine from each subject, samples were analyzed for ¹⁴C content using Accelerator Mass Spectrometry (AMS) to determine the maximum concentration of M₄N (Cₘₐₓ) that occurred at time Tₘₐₓ, the overall area under the curve (AUC) that corresponds to the overall absorption of M₄N at the times of testing (AUC₀₋ₜ) and overall (AUC_{0-∞}), the terminal half-life (T_{1/2}) of each sample and the relative and overall bioavailability (Fᵣₑₗ and F), of the oral dose of M₄N compared to the IV dose of M₄N. The mean±SD values of pharmacokinetic parameters for ¹⁴C are presented in Table 24. The baseline levels of M₄N were corrected for any residual M₄N remaining in the subjects following the periods between dosing so as not to inflate the levels of M₄N for any subsequent dosing.

TABLE 24
Mean ± SD values of pharmacokinetic parameters
for ¹⁴C (Baseline Corrected)

| | Regimen A | Regimen B | Regimen C |
|---|---|---|---|
| Parameter | (oral, fed) | (oral, fasted) | (Intravenous) |
| Cₘₐₓ (pmol/L) | 5.94 ± 1.33 | 9.29 ± 1.40 | 10.31 ± 1.77 |
| Tₘₐₓ (hours) | 2.50^{a} | 1.00^{a} | 0.08^{a} |
| AUC₀₋ₜ (pmol · h/L) | 88.0 ± 19.4 | 117.2 ± 9.2 | 96.6 ± 9.23 |
| AUC_{0-∞} (pmol · h/L) | 625.6 ± 183.7 | 416.6 ± 142.6 | 707.7 ± 380.4 |
| T½ (hours) | 96.5 ± 20.7 | 50.9 ± 22.4 | 116.2 ± 64.0 |
| Fᵣₑₗ (%) | 75.1 ± 16.2 | — | — |
| F (%) | 91.2 ± 19.1 | 122.1 ± 14.5 | — |

^{a}Median
Regimen A: 100 μg ¹⁴C-labelled M₄N (3.3 kBq) administered as a single oral dose following a high fat breakfast.
Regimen B: 100 μg ¹⁴C-labelled M₄N (3.3 kBq) administered as a single oral dose following an overnight fast.
Regimen C: 100 μg ¹⁴C-labelled M₄N (3.3 kBq) administered as 1 mL bolus intravenous solution following an overnight fast.

For the oral doses, the Cₘₐₓ values for total 14C were lower following oral administration of 100 μg ¹⁴C-labelled M₄N after a high fat breakfast (Cₘₐₓ=5.94±1.33 pmol/L) than following oral administration after an overnight fast (Cₘₐₓ=9.29±1.40 pmol/L). Tₘₐₓ tended to occur later in fed subjects than in fasted subjects. In fed subjects, Tₘₐₓ, the time of occurrence of Cₘₐₓ, was highly variable and ranged from 1.5 to 24 hours post-dose, and in fasted subjects Tₘₐₓ generally occurred at 1 hour post-dose (range 0.50 to 2.00 hours). The AUC₀₋ₜ values were generally lower in fed subjects than in fasted subjects.

Following the intravenous dose the maximum concentration (Cₘₐₓ=10.31±1.77 pmol/L) occurred, as expected, at the first sampling time (0.08 hours post-dose) in eight of the ten subjects. In two subjects, Tₘₐₓ was 0.17 hours post-dose. The AUC₀₋ₜ values for total ¹⁴C plasma concentrations were slightly lower following the single oral dose in fed subjects than following the intravenous dose. Conversely, the corresponding AUC₀₋ₜ values were slightly higher following the single oral dose in the fasted subjects than following the intravenous dose.

The study formulations were well tolerated in both oral and intravenous administrations. There were no serious or severe adverse events and no subjects discontinued because of a study treatment related adverse event. No clinically significant changes in vital signs or ECGs were seen.

In conclusion regarding this study, the apparent absorption of M₄N was very high following oral administration in the fed and fasted state. In the presence of food, the rate and extent of absorption were lower compared with the fasted state and the time of occurrence of Cₘₐₓ was prolonged in the fed state. These conclusions are made on the assumption that the orally administered doses of ¹⁴C-labelled M₄N were not degraded prior to absorption.

### EXAMPLE 14

### Additional Studies of Solubility of M₄N in Water-Soluble Organic Solvents

The solubility of M₄N in combinations of water-soluble organic solvents as noted in Table 25 was evaluated up to 48 hours. Following 2, 24 and 48 hours incubation at room temperature samples were analyzed via Reverse Phase-HPLC (“RP-HPLC”) to quantify the solubility of M₄N. To prepare M₄N samples, 200 μL of 100 mg/mL M₄N dissolved in acetone were placed in 1.5 mL polypropylene microtubes. The solvent was allowed to evaporate at room temperature for 48 hours until the samples were completely dry.

The water miscible organic solvent formulations were prepared in 15 mL polypropylene centrifuge tubes. 10 mL of each formulation was prepared. Each solvent was added on the basis of weight using its respective density at 25° C. Following brief mixing, each formulation was filtered through a 0.45 μm surfactant free cellulose acetate (“SFCA”) filter into a fresh 15 mL tube. Formulations were kept at room temperature until ready for use.

400 μL of each formulation combination (Table 25, where Benz=benzyl alcohol; Crem=Cremophor® EL; DMA=dimethylacetamide; T80=Tween® 80) were added to the microtube, enabling a maximum solubility of 50 mg/Ml M₄N. The M₄N solubility was evaluated by RP-HPLC at 2, 24 and 48 hours incubation at room temperature. At each time point, the samples were centrifuged for 2 minutes at 13,000 rpm to pellet any solid M₄N. As noted in Table 25, over half of the formulation conditions examined were able to solubilize M₄N to a concentration of greater than 10 mg/Ml. The solubility of M₄N in glycerol at 2 and 48 hours was not detectable.

TABLE 25
M₄N solubility in water miscible organic solvents up to 48 hours

| | M₄N (mg/Ml) | | |
|---|---|---|---|
| | Time (hrs) | | |
| Composition | 2 | 24 | 48 |
| 100% EtOH | 7.20 | 7.22 | 7.91 |
| 100% PG | 1.10 | 1.33 | 1.76 |
| 100% PEG300 | 5.81 | 10.37 | 11.52 |
| 100% Glycerol | XXX | 0.08 | XXX |
| 100% Crem | 1.19 | 7.51 | 12.48 |
| 50% EtOH, 50% PG | 3.64 | 4.15 | 4.43 |
| 50% EtOH, 50% PEG300 | 10.94 | 15.07 | 16.61 |
| 50% EtOH, 50% Glycerol | 1.14 | 1.53 | 1.60 |
| 50% EtOH, 50% Crem | 13.95 | 17.58 | 18.49 |
| 50% EtOH, 50% T80 | 15.91 | 19.28 | 19.68 |
| 50% PG, 50% PEG300 | 2.65 | 4.88 | 5.30 |
| 50% PG, 50% Glycerol | 0.13 | 0.48 | 0.51 |
| 50% PG, 50% Crem | 2.94 | 6.33 | 7.20 |
| 50% PG, 50% T80 | 5.07 | 8.16 | 8.41 |
| 48% EtOH, 50% PG, 2% Benz | 4.42 | 4.79 | 4.92 |
| 48% EtOH, 50% PEG300, 2% Benz | 14.51 | 15.78 | 16.49 |
| 48% EtOH, 50% Glycerol, 2% Benz | 1.25 | 1.66 | 1.73 |
| 48% EtOH, 50% Crem, 2% Benz | 14.02 | 18.17 | 18.51 |
| 48% EtOH, 50% T80, 2% Benz | 16.17 | 19.55 | 19.96 |
| 44% EtOH, 50% PG, 6% DMA | 4.80 | 5.48 | 2.93 |
| 44% EtOH, 50% PEG300, 6% DMA | 15.12 | 18.61 | 18.27 |
| 44% EtOH, 50% Glycerol, 6% DMA | 1.43 | 1.90 | 2.01 |
| 44% EtOH, 50% Crem, 6% DMA | 14.85 | 20.42 | 21.08 |
| 44% EtOH, 50% T80, 6% DMA | 17.72 | 22.41 | 23.00 |
| 18% EtOH, 30% PG, 40% PEG300, | 7.84 | 9.79 | 10.16 |
| 10% T80, 2% Benz | | | |
| 18% EtOH, 30% PG, 40% Glyc, | 0.58 | 1.01 | 1.15 |
| 10% T80, 2% Benz | | | |
| 18% EtOH, 30% PG, 40% Crem, | 8.64 | 11.78 | 11.86 |
| 10% T80, 2% Benz | | | |
| 14% EtOH, 30% PG, 40% PEG300, | 9.40 | 10.55 | 11.30 |
| 10% T80, 6% DMA | | | |
| 14% EtOH, 30% PG, 40% Glyc, | 0.85 | 1.24 | 1.48 |
| 10% T80, 6% DMA | | | |
| 14% EtOH, 30% PG, 40% Crem, | 9.03 | 12.08 | 12.28 |
| 10% T80, 6% DMA | | | |
| 28% EtOH, 30% PG, 30% PEG300, | 8.87 | 10.19 | 10.27 |
| 10% T80, 2% Benz | | | |
| 28% EtOH, 30% PG, 30% Glyc, | 1.86 | 2.22 | 2.53 |
| 10% T80, 2% Benz | | | |
| 28% EtOH, 30% PG, 30% Crem, | 8.98 | 11.35 | 11.28 |
| 10% T80, 2% Benz | | | |
| 24% EtOH, 30% PG, 30% PEG300, | 9.91 | 10.80 | 10.67 |
| 10% T80, 6% DMA | | | |
| 24% EtOH, 30% PG, 30% Glyc, | 1.63 | 2.26 | 2.52 |
| 10% T80, 6% DMA | | | |
| 24% EtOH, 30% PG, 30% Crem, | 10.42 | 11.25 | 12.48 |
| 10% T80, 6% DMA | | | |

### EXAMPLE 15

### M₄N Solubility in Aqueous Solutions

The solubility of M₄N in aqueous solutions containing either hydroxypropyl HP-β-CD or sulfobutyl ether β-cyclodextrin (SE-β-CD) (Captisol®, CyDex, Inc., Lenexa, Kans., U.S.A.) was evaluated up to 48 hours at room temperature described above in Example 14. Ten 50% solutions of HP-β-CD and SE-β-CD were prepared on a weight to volume basis. The M₄N for use in the samples was prepared as set forth in Example 14. Between 1.0 g and 5.0 g of either compound was weighed into a 10 mL volumetric flask on an OHAUS Analytical Plus Balance. Each sample was q.s. to 10 mL with water for injection (WFI). Following a 1 hour incubation at 40° C., the preparations were filtered through a 0.45 μm SFCA filter into a fresh 15 mL tube. Preparations were kept at room temperature until ready for use.

As shown in Table 26, the solubility of M₄N in WFI, 0.9% saline, 5% dextrose (D5W) was below the quantitation limit of the RP-HPLC method throughout the course of this study. Note the increased M₄N solubility as a function of HP-β-CD and Captisol® concentration and time.

TABLE 26
M₄N solubility in aqueous solutions up to 48 hours

| | M₄N (mg/mL) | | |
|---|---|---|---|
| | Time (hrs) | | |
| Composition | 2 | 24 | 48 |
| WFI | XXX | XXX | XXX |
| 0.9% Saline | XXX | XXX | XXX |
| D5W | XXX | XXX | XXX |
| 10% HP-β-CD | 0.35 | 0.45 | 0.46 |
| 20% HP-β-CD | 0.66 | 0.91 | 0.88 |
| 30% HP-β-CD | 1.38 | 1.97 | 2.02 |
| 40% HP-β-CD | 1.89 | 3.01 | 3.23 |
| 50% HP-β-CD | 2.52 | 4.95 | 4.98 |
| 10% Captisol ® | 0.48 | 0.74 | 0.94 |
| 20% Captisol ® | 0.76 | 1.45 | 1.39 |
| 30% Captisol ® | 1.57 | 2.87 | 2.69 |
| 40% Captisol ® | 1.42 | 3.90 | 4.15 |
| 50% Captisol ® | 1.17 | 4.49 | 6.41 |

More than 20 formulation conditions using water miscible organic solvents were able to solubilize M₄N to a concentration of greater than 10 mg/mL. The solubility of M₄N in WFI, 0.9% saline, D5W was below the detection limit of the RP-HPLC method. The solubility of M₄N increases as a function of HP-β-CD and Captisol® concentration and time.

### EXAMPLE 16

### M₄N Solubility in Hydroxypropyl β-Cyclodextrin

The aqueous solubility of M₄N at various concentrations of HP-β-CD was evaluated by the method reported by Higuchi and Connors (1965). Briefly, M₄N was accurately weighed and added in quantities exceeding its aqueous solubility were gently rotated (12 rpm) at room temperature with aqueous solutions of HP-β-CD in increasing concentrations (0-350 mmol/L), for a period of 48 hours. The M₄N/HP-β-CD solutions were then filtered through a 0.45 μm SFCA filter and analyzed via RP-HPLC.

Although most drug/cyclodextrin complexes are thought to be inclusion complexes, cyclodextrins are also known to form non-inclusion complexes and complex aggregates capable of dissolving drugs through micelle-like structures. The phase-solubility profiles did not verify formation of inclusion complexes, but only detail how the increasing concentration of cyclodextrin influences drug solubility. Formation of the M₄N/HP-β-CD complex is non-linear, but accurate determination of stoichiometry (as well as stability constants) was not studied in the experiments of this Example, but could be determined by other means such as NMR or potentiometry.

### EXAMPLE 17

### M₄N Stability in HP-β-CD/PEG 300 Buffer Solutions

The stability of 10 mg/mL M₄N (prepared in a ratio of 75:25 40% HP-β-CD: 40 mg/mL M₄N PEG 300) in 15 mM buffer solutions was evaluated following incubation at 60° C.

Buffered 40% solutions of HP-β-CD were prepared on a weight to volume basis. The M₄N for use in the samples was prepared as set forth in Example 14. 2.0 g HP-β-CD was weighed into 5 mL volumetric flasks on an OHAUS Analytical Plus Balance. 1 mL of a 100 mM buffer solution was added to each flask. Each sample was q.s. to 5 mL with WFI. Following a 1 hour incubation at 40° C., the preparations were filtered through a 0.45 μm SFCA filter into a fresh 15 mL tube. Preparations were kept at room temperature until ready for use.

750 μL 40% HP-β-CD buffered solution was placed in 1.5 mL polypropylene microtubes. 250 μL of a 40 mg/mL M₄N PEG 300 was added to each microtube enabling a solubility of 10 mg/mL M₄N. After gentle inversion of the sample tubes, the pH of each solution was measured using a Orion, Model 420A pH meter. An initial aliquot was removed for RP-HPLC analysis. Test samples were then placed in a Precision 60° C. incubator. The M₄N stability was evaluated by RP-HPLC. At each time point, the samples were centrifuged for 2 minutes at 13,000 rpm to pellet any solid M₄N.

As shown in Table 27, a slight decrease in the concentration of the various M₄N solutions is observed following the 14 day incubation at 60° C. RP-HPLC data did not reveal an increase in sample impurities which could account for the magnitude of decrease in the concentration of M₄N. However, a pH-dependent change was observed in the M₄N impurities, yet these impurities made up less than 0.1% of the total peak area. Little if any changes are observed in the apparent sample pH during the incubation period (Table 27).

TABLE 27
M₄N stability in HP-β-CD/PEG solutions up to 14 days at 60° C.

| | M₄N (mg/mL) | | | | |
|---|---|---|---|---|---|
| | Time (days) | | | | |
| Composition | 0 | 2 | 6 | 10 | 14 |
| WFI, 30% HP-β-CD, 25% PEG300 | 10.4 | 10.1 | 9.9 | 9.7 | 9.6 |
| 15 mM Phosphate, pH 3 30% HP-β-CD, | 10.3 | 10.1 | 9.6 | 9.7 | 9.6 |
| 25% PEG300 | | | | | |
| 15 mM Phosphate, pH 4 30% HP-β-CD, | 10.5 | 10.1 | 9.6 | 9.7 | 9.6 |
| 25% PEG300 | | | | | |
| 15 mM Acetate, pH 5 30% HP-β-CD, | 10.6 | 10.2 | 9.6 | 9.7 | 9.6 |
| 25% PEG300 | | | | | |
| 15 mM Acetate, pH 6 30% HP-β-CD, | 10.5 | 10.1 | 9.7 | 9.7 | 9.6 |
| 25% PEG300 | | | | | |
| 15 mM Phosphate, pH 7 30% HP-β-CD, | 10.5 | 10.3 | 9.7 | 9.7 | 9.7 |
| 25% PEG300 | | | | | |
| 15 mM Phosphate, pH 8 30% HP-β-CD, | 10.6 | 10.4 | 9.7 | 9.8 | 9.6 |
| 25% PEG300 | | | | | |
| 15 mM Phosphate, pH 9 30% HP-β-CD, | 10.4 | 10.2 | 9.7 | 9.8 | 9.7 |
| 25% PEG300 | | | | | |
| 15 mM Borate, pH 10 30% HP-β-CD, | 10.5 | 10.1 | 9.7 | 9.6 | 9.6 |
| 25% PEG300 | | | | | |
| 15 mM Borate, pH 11 30% HP-β-CD, | 10.5 | 10.3 | 9.8 | 9.5 | 9.4 |
| 25% PEG300 | | | | | |

A uniform decrease in stability regardless of apparent sample pH or buffer, as indicated by a loss in the recovery of M₄N, was observed after 14 days of incubation at 60° C.

### EXAMPLE 18

### M₄N Stability in 11-14 mg/mL PEG 300/HP-β-CD Solutions

To support manufacturing specifications, this study examined the 24-hour room temperature stability of combinations of PEG 300/HP-β-CD at varying M₄N target concentrations. Stock samples of M₄N stocks were prepared in 100% PEG 300 at drug concentrations of 33-56 mg/mL at 60° C. as follows.

M₄N bulk drug was solubilized to concentrations of 33, 44, 48, 52, 55 and 56 mg/mL (w/w) in PEG 300 using the procedure set forth in Examples 14 and 17, following incubation of at least 2 hours at 60° C. Vigorous vortexing and mixing were necessary for complete solubilization of M₄N above 44 mg/mL. The M₄N stock solutions were filtered through a 0.45 μm SFCA filter and used within 30 minutes of preparation. Separately, a solution of 40% (w/v) HP-β-CD was prepared in sterile WFI and filtered. Combinations of the 40% HP-β-CD stock and the M₄N/PEG 300 stocks were combined in 1.5 mL polypropylene microtubes. The M₄N solubility was evaluated by RP-HPLC at 2 and 24 hours incubation at room temperature.

The requisite amount of M₄N stock was added to 40% HP-β-CD to yield the final concentrations of drug and excipients listed in Table 28. Samples were gently rotated (˜12 rpm) at room temperature. At 2 and 24 hours of incubation, samples were centrifuged 2 minutes at 13,000 rpm and 50 μL aliquots were removed for RP-HPLC analysis. Regardless of target M₄N or formulation, little if any changes in solubility were observed after the 24 hour incubation (Table 28).

TABLE 28
M₄N Stability in 11-14 mg/mL PEG300/HP-β-CD solutions

| | Observed M₄N mg/mL | |
|---|---|---|
| | | t = 24 |
| Composition | t = 2 hours | hours |
| 11 mg/mL M₄N, 25% PEG300, 30% HP-β-CD | 11.3 | 11.3 |
| 12 mg/mL M₄N, 25% PEG300, 30% HP-β-CD | 11.8 | 11.6 |
| 13 mg/mL M₄N, 25% PEG300, 30% HP-β-CD | 12.7 | 12.7 |
| 14 mg/mL M₄N, 25% PEG300, 30% HP-β-CD | 13.5 | 13.5 |
| 11 mg/mL M₄N, 33% PEG300, 27% HP-β-CD | 11.4 | 11.3 |
| 11 mg/mL M₄N, 20% PEG300, 32% HP-β-CD | 11.5 | 11.4 |

Test samples containing between 11-14 mg/mL M₄N formulated to a final concentration of 25% PEG 300 and 30% HP-β-CD were stable after 24 hours incubation at room temperature.

### EXAMPLE 19

### 40 mg/mL M₄N/PEG 300 Stability

The stability of 40 mg/mL M₄N dissolved in 100% PEG 300 was evaluated up to 24 hours incubation at 30° C., 45° C. and 60° C. A stock 40 mg/mL M₄N in PEG 300 was prepared at 60° C., following the procedure set forth in Example 18. Subsequently, aliquots were removed and incubated at the appropriate temperature. Samples were rotated at 450 rpm during the entire course of incubation. Visual observations and RP-HPLC data were collected throughout. As shown in Table 29, after 6 hours incubation at 30° C. tiny crystals were observed in the 40 mg/mL M₄N/PEG 300 formulation, with more appearing after 24 hours. The formation of crystals coincided with a loss of soluble M₄N (Table 30). The 40 mg/mL M₄N/PEG 300 samples incubated at 45° C. and 60° C. were stable after 24 hours incubation as assessed by visual observations and RP-HPLC analysis (Tables 29 and 30). No changes in the amount or types of impurities peaks were observed at any of the incubation temperatures.

TABLE 29
Visual appearance of 40 mg/mL M₄N/PEG 300 stability samples

| | |
|---|---|
| | Visual Appearance |
| Incubation | Time (hours) |

| Condition | 2 | 4 | 6 | 24 |
|---|---|---|---|---|
| 30° C. | Clear | Clear | Few tiny | Many tiny |
| | | | crystals | crystals |
| 45° C. | Clear | Clear | Clear | Clear |
| 60° C. | Clear | Clear | Clear | Clear |

TABLE 30
40 mg/mL M₄N/PEG 300 stability samples: RP-HPLC analysis

| | |
|---|---|
| | M₄N (mg/mL) |
| Incubation | Time (hours) |

| Condition | 0 | 2 | 6 | 24 |
|---|---|---|---|---|
| 30° C. | 39.5 | 40.8 | 40.6 | 37.4 |
| 45° C. | | 40.4 | 39.8 | 40.6 |
| 60° C. | | 39.1 | 39.2 | 39.2 |

After 6 hours incubation at 30° C. tiny crystals were observed in the 40 mg/mL M₄N/PEG 300 formulation. After 24 hours, even more crystals were observed as well as a >5% loss in soluble M₄N as determined by RP-HPLC analysis.

40 mg/mL M₄N/PEG 300 samples incubated at 45° C. and 60° C. were stable after 24 hours incubation as assessed by visual observations and RP-HPLC analysis.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

### BIBLIOGRAPHY

Ansel, H. C. et al. (2004). Pharmaceutical Dosage Forms and Drug Delivery Systems eds., 8^{th} ed., Lippincott Williams & Wilkins.
Garg, S. et al. (2001). Compendium of Pharmaceutical Excipients for Vaginal Formulations. Pharmaceutical Technol. Drug Delivery. Sep. 1, 2001, pp. 14-24.
Gennaro, A. R. (2003). Remington: The Science and Practice of Pharmacy, 20^{th} ed., with Facts and Comparisons: DrugfactsPlus. Lippincott Williams & Williams.
Higuchi T and Connors K A (1965) “Phase solubility techniques”, Adv Anal Chem Instr. 4, 117-212.
Hwu, J. R. et al. (1998). Antiviral activities of methylated nordihydroguairetic acids. 1. Synthesis, structure identification, and inhibition of Tat-regulated HIV transactivation. J. Med. Chem. 41: 2994-3000.
McDonald, R. W. et al. (2001). Synthesis and anticancer activity of nordihydroguairetic acid (NDGA) and analogues. Anti-Cancer Drug Design 16: 261-270.
Rowe, R. C. et al. eds. (2003). Handbook of Pharmaceutical Excipients. 4^{th} edition. Pharmaceutical Press and American Pharmaceutical Association.

We claim:

1. A composition for oral administration to an animal comprising an active pharmaceutical ingredient and a pharmaceutically acceptable carrier, wherein the active pharmaceutical ingredient comprises a catecholic butane, and the carrier comprises at least one of a solubilizing agent and an excipient selected from the group consisting of: (a) a water-soluble organic solvent other; provided that when the water-soluble organic solvent is propylene glycol, the propylene glycol is in the absence of white petrolatum, in the absence of xanthan gum and in the absence of at least one of glycerine or glycine, when the water-soluble organic solvent is polyethylene glycol, the polyethylene glycol is present in the absence of ascorbic acid or butylated hydroxytoluene, and when the polyethylene glycol is polyethylene glycol 400, the polyethylene glycol 400 is present in the absence of polyethylene glycol 8000; (b) a cyclodextrin; (c) an ionic, non-ionic or amphipathic surfactant, provided that when the surfactant is a non-ionic surfactant, the non-ionic surfactant is present in the absence of xanthan gum; (d) a modified cellulose; (e) a water-insoluble lipid, provided that when the water-insoluble lipid is castor oil, the castor oil is present in the absence of beeswax or carnuba wax; and a combination of any of the carriers (a)-(e).

2. The composition of claim 1, wherein the composition comprises about 0.1 mg to about 200 mg of the active pharmaceutical ingredient.

3. The composition of claim 2, wherein the composition comprises about 10 mg, about 20 mg, about 25 mg, about 30 mg, about 40 mg, about 50 mg, about 60 mg, about 75 mg, about 100 mg or about 200 mg of the active pharmaceutical agent.

4. The composition of claim 1, wherein the active pharmaceutical ingredient is present at a concentration of about 1 mg/mL to about 200 mg/mL or about 1 mg/g to about 250 mg/g.

5. The composition of claim 4, wherein the active pharmaceutical ingredient is present at a concentration of about 1 mg/mL, about 2 mg/mL, about 2.5 mg/mL, about 5 mg/mL, about 10 mg/mL, about 12.5 mg/mL, about 15 mg/mL, about 20 mg/mL, about 25 mg/mL, about 30 mg/mL, about 40 mg/mL, about 50 mg/mL, about 55 mg/mL, about 60 mg/mL, about 75 mg/mL, about 100 mg/mL, about 125 mg/mL, about 150 mg/mL or about 175 mg/mL.

6. The composition of claim 4, wherein the active pharmaceutical ingredient is present at a concentration of about 20 mg/g, about 50 mg/g, about 75 mg/g, about 100 mg/g, about 120 mg/g, about 130 mg/g, about 140 mg/g, about 150 mg/g, about 175 mg/g or about 200 mg/g.

7. The composition of claim 1, wherein the water-soluble organic solvent is selected from the group consisting of polypropylene glycol, polyethylene glycol, polyvinyl pyrrolidone, ethyl alcohol, benzyl alcohol and dimethylacetamide.

8. The composition of claim 1, wherein the carrier comprises polyethylene glycol.

9. The composition of claim 8, wherein the polyethylene glycol is present at a concentration of about 5% (v/v) to about 100% (v/v).

10. The composition of claim 9, wherein the polyethylene glycol is present at a concentration of about 20% (v/v) to about 80% (v/v).

11. The composition of claim 10, wherein the polyethylene glycol is present at a concentration of about 50% (v/v).

12. The composition of claim 10, wherein the polyethylene glycol is present at a concentration of about 40% (v/v).

13. The composition of claim 10, wherein the polyethylene glycol is present at a concentration of about 33% (v/v).

14. The composition of claim 8, wherein the polyethylene glycol is PEG 300.

15. The composition of claim 14, wherein the PEG 300 is present at a concentration of about 10% (v/v), about 20% (v/v), about 30% (v/v), about 40% (v/v) or about 50% (v/v).

16. The composition of claim 8, wherein the polyethylene glycol is PEG 400.

17. The composition of claim 16, wherein the PEG 400 is present at a concentration of about 10% (v/v), about 20% (v/v), about 30% (v/v), about 40% (v/v) or about 50% (v/v).

18. The composition of claim 8 wherein the polyethylene glycol is PEG 400 monolaurate.

19. The composition of claim 18, wherein the PEG 400 monolaurate is present at a concentration of about 20% (v/v) to about 50% (v/v).

20. The composition of claim 1 or 8, wherein the carrier comprises an unmodified cyclodextrin or a modified cyclodextrin.

21. The composition of claim 20, wherein the modified cyclodextrin is selected from the group consisting of hydroxypropyl-β-cyclodextrin and sulfobutyl ether β-cyclodextrin.

22. The composition of claim 20, wherein the modified cyclodextrin is present at a concentration of about 5% (w/v) to about 80% (w/v).

23. The composition of claim 22, wherein the modified cyclodextrin is present at a concentration of about 15% (w/v), about 20% (w/v), about 25% (w/v), about 30% (w/v), about 35% (w/v), about 40% (w/v) or about 50% (w/v).

24. The composition of claim 1, wherein the carrier comprises a surfactant.

25. The composition of claim 24, wherein the surfactant is present at a concentration of about 5% (v/v) to about 100% (v/v).

26. The composition of claim 25, wherein the surfactant is present at a concentration of about 30% (v/v), about 40% (v/v) or about 50% (v/v).

27. The composition of claim 1, wherein the carrier comprises a surfactant selected from the group consisting of polysorbate, d-alpha-tocopheryl polyethylene glycol 1000 succinate, an esterified fatty acid, and the reaction product of ethylene oxide and castor oil in a 35:1 molar ratio.

28. The composition of claim 26, wherein the surfactant is selected from the group consisting of polysorbate 20 and polysorbate 80.

29. The composition of claim 28, wherein the surfactant is polysorbate 20.

30. The composition of claim 1, wherein the carrier comprises polyethylene glycol and a surfactant.

31. The composition of claim 30, wherein the polyethylene glycol is selected from the group consisting of PEG 300 and PEG 400.

32. The composition of claim 31, wherein the surfactant is polysorbate 20.

33. The composition of claim 24, wherein the surfactant is an esterified fatty acid.

34. The composition of claim 1, wherein the carrier comprises a modified cellulose.

35. The composition of claim 34, wherein the modified cellulose is selected from the group consisting of ethyl cellulose, hydroxypropyl methylcellulose, methylcellulose and carboxy methylcellulose.

36. The composition of claim 34, wherein the modified cellulose is present at a concentration of about 0.1% (w/v) to about 10% (w/v).

37. The composition of claim 1, wherein the carrier comprises a water-insoluble lipid.

38. The composition of claim 37, wherein the water-insoluble lipid is selected from the group consisting of at least one of an oil, a wax and a fat emulsion, provided that when the composition contains castor oil, it does not contain beeswax or carnuba wax.

39. The composition of claim 37, wherein the water-insoluble lipid is a fat emulsion.

40. The composition of claim 39, wherein the fat emulsion is present at a concentration of about 10% to about 30%.

41. The composition of claim 39, wherein the fat emulsion is present at a concentration of about 20%.

42. The composition of claim 33, wherein the water-insoluble lipid is oil.

43. The composition of claim 42, wherein the oil is selected from at least one of the group consisting of corn oil, olive oil, peppermint oil, soy bean oil, sesame seed oil, mineral oil and glycerol.

44. The composition of claim 42, wherein the oil is present at a concentration of about 10% to about 100%.

45. The composition of claim 42, wherein the oil is peppermint oil.

46. The composition of claim 45, further comprising sesame oil.

47. The composition of claim 42, further comprising polysorbate 20.

48. The composition of claim 47, further comprising a polyethylene glycol.

49. The composition of claim 42, further comprising a polyethylene glycol.

50. The composition of claim 49, polyethylene glycol is selected from the group consisting of PEG 300 and PEG 400.

51. The composition of claim 49, polyethylene glycol is selected from the group consisting of PEG 300 and PEG 400.

52. The composition of claim 1, wherein the catecholic butane has a structural formula of Formula I: 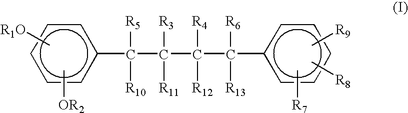
wherein R₁ and R₂ each independently represents —H, a lower alkyl, a lower acyl, an alkylene; or —R₁O and —R₂O each independently represents an unsubstituted or substituted amino acid residue or salt thereof; R₃, R₄, R₅, R₆, R₁₀, R₁₁, R₁₂ and R₁₃ each independently represents —H or a lower alkyl; and R₇, R₈, and R₉ each independently represents —H, —OH, a lower alkoxy, a lower acyloxy, an unsubstituted or substituted amino acid residue or a salt thereof, or any two adjacent groups together may be an alkylene dioxy.

53. The composition of claim 1, wherein the catecholic butane is a NDGA compound.

54. The composition of claim 53, wherein the NDGA compound has a structural formula of Formula II: 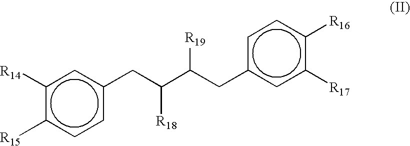
wherein R₁₄, R₁₅, R₁₆ and R₁₇ each independently represents —OH, a lower alkoxy, a lower acyloxy, or an unsubstituted or substituted amino acid residue or pharmaceutically acceptable salt thereof; and R₁₈ and R₁₉ each independently represents —H or a lower alkyl.

55. The composition of claim 54, wherein R₁₄, R₁₅, R₁₆ and R₁₇ each independently represents a lower alkoxy.

56. The composition of claim 54, wherein R₁₄, R₁₅, R₁₆ and R₁₇ each independently represents —OCH₃.

57. The composition of claim 54, wherein R₁₄, R₁₅, R₁₆ and R₁₇ each independently represents a lower acyloxy.

58. The composition of claim 54, wherein R₁₄, R₁₅, R₁₆ and R₁₇ each independently represents —O(C═O)CH₃.

59. The composition of claim 54, wherein R₁₄, R₁₅, R₁₆ and R₁₇ each independently represents a substituted amino acid residue.

60. The composition of claim 54, wherein R₁₄, R₁₅, R₁₆ and R₁₇ each independently represents a N,N-dimethyl-substituted amino acid residue.

61. The composition of claim 54, wherein R₁₄, R₁₅, R₁₆ and R₁₇ each independently represents a salt of a substituted amino acid residue.

62. The composition of claim 54, wherein R₁₄, R₁₅, R₁₆ and R₁₇ each independently represents a chloride salt of a substituted amino acid residue.

63. The composition of claim 54, wherein R₁₄, R₁₅, R₁₆ and R₁₇ each independently represents a substituted amino acid residue or a salt thereof, and the substituted amino acid residue or salt is —O(C═O)CH₂N(CH₃)₂ or —O(C═O)CH₂N⁺(CH₃)₂.Cl⁻.

64. The composition of claim 54, wherein R₁₈ and R₁₉ each independently represents a lower alkyl.

65. The composition of claim 54, wherein R₁₈ and R₁₉ each independently represents —CH₃.

66. The composition of claim 54, wherein R₁₄, R₁₅, R₁₆ and R₁₇ are not each —OH simultaneously.

67. The composition of claim 53, wherein the NDGA compound is a methylated derivative of NDGA.

68. The composition of claim 67, wherein the NDGA compound is selected from the group consisting of tetra-O-methyl NDGA (M₄N), tri-O-methyl NDGA (M₃N), di-O-methyl NDGA (M₂N) and mono-O-methyl NDGA (MIN).

69. The composition of claim 1, wherein the active pharmaceutical ingredient is tetra-O-methyl NDGA.

70. A method of treatment of a disease in a subject comprising: (a) providing the composition of claim 1; and (b) administering the composition orally to the subject, wherein the composition comprises an effective amount of the active pharmaceutical ingredient.

71. The method of claim 70, wherein the disease is a proliferative disease.

72. The method of claim 71, wherein the proliferative disease is cancer.

73. The method of claim 71, wherein the proliferative disease is psoriasis.

74. The method of claim 70, wherein the disease is hypertension.

75. The method of claim 76, wherein the disease is obesity.

76. The method of claim 70, wherein the disease is diabetes.

77. The method of claim 70, wherein the disease is a central nervous system disease or a neurodegenerative disease.

78. The method of claim 70, wherein the disease is pain.

79. The method of claim 70, wherein the disease is Alzheimer's disease, amyotrophic lateral sclerosis, dementia or Parkinson's disease.

80. The method of claim 70, wherein the disease is stroke.

81. The method of claim 70, wherein the disease is an inflammatory disease.

82. The method of claim 81, wherein the inflammatory disease is selected from the group consisting of rheumatoid arthritis, osteoarthritis, ulcerative colitis, Crohn's disease, atherosclerosis, chronic obstructive pulmonary disease (COPD) and multiple sclerosis.

83. The method of claim 70, wherein the disease is premalignant neoplasia or dysplasia.

84. The method of claim 83, wherein the disease is an intraepithelial neoplasia.

85. The method of claim 70, wherein the disease is an infection.

86. The method of claim 70, wherein the infection is a viral infection.

87. The method of claim 86, wherein the virus is selected from the group consisting of HIV, HTLV, HPV, HSV, HBV, EBV, Varicella-zoster virus, adenovirus, parvovirus or JC virus.

88. The method of claim 70, wherein the composition is administered at a dose in a range of about 10 mg of active pharmaceutical ingredient per kg weight of the subject to about 600 mg of active pharmaceutical ingredient per kg weight of the subject.

89. The method of claim 70, wherein the composition is administered one or more times per week.

90. The method of claim 70, wherein the composition is administered one or more times per month.

91. A kit for treatment of a disease comprising the composition of claim 1 and instructions for use thereof.
